(19) United States
(12) Patent Application Publication
(10) Pub. No.: US 2008/0312286 A1
(43) Pub. Date: Dec. 18, 2008

(54) Indanone Potentiators of Metabotropic Glutamate Receptors

(76) Inventors: Anthony B. Pinkerton, San Diego, CA (US); Jean-Michel Vernier, San Diego, CA (US); Rowena Cube, Somerset, PA (US); John Hutchinson, La Jolla, CA (US); Celine Bonnefous, San Diego, CA (US); Theodore Kamenecka, Palm Beach Gardens, FL (US)

Correspondence Address: MERCK AND CO., INC, P O BOX 2000, RAHWAY, NJ, 07065-0907, US

(21) Appl. No.: 11/658,490

(22) PCT Filed: Jul. 26, 2005
(86) PCT No.: PCT/US05/26867

§ 371 Date: Jan. 24, 2007

Related U.S. Application Data
(60) Provisional application No. 60/592,549, filed on Jul. 30, 2004.

(51) Int. Cl. C07D 257/04; A61K 31/41; C07C 307/02; A61K 31/18; C07C 233/02; A61K 31/454; C07D 401/02; A61K 31/192; C07C 65/00; C07D 213/80; A61K 31/44; A61K 31/343; C07D 307/78; A61P 25/00; A61P 25/18; A61P 25/24; A61P 25/08

(52) U.S. Cl. 514/340; 548/252; 514/381; 564/99; 514/605; 514/616; 564/158; 546/268.4; 514/569; 562/466; 546/322; 514/356; 514/469; 549/469

(57) ABSTRACT

The present invention is directed to compounds which are potentiators of metabotropic glutamate receptors, including the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The excitatory amino acid L-glutamate (sometimes referred to herein simply as glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception.

Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life. Schoepp, Bockaert, and Sladeczek, Trends in Pharmacol. Sci., 11, 508 (1990); McDonald and Johnson, Brain Research Reviews, 15, 41 (1990).

The present invention relates to potentiators of mGlu receptors, in particular mGluR2 receptors. The mGluR receptors belong to the Type III G-protein coupled receptor (GPCR) superfamily. This superfamily of GPCR'sf including the calcium-sensing receptors, GABAB receptors and pheromone receptors, which are unique in that they are activated by binding of effectors to the amino-terminus portion of the receptor protein. The mGlu receptors are thought to mediate glutamate's demonstrated ability to modulate intracellular signal transduction pathways. Ozawa, Kamiya and Tsuzuski, Prog. Neurobio., 54, 581 (1998). They have been demonstrated to be localized both pre- and post-synaptically where they can regulate neurotransmitter release, either glutamate or other neurotransmitters, or modify the post-synaptic response of neurotransmitters, respectively.

At present, there are eight distinct mGlu receptors that have been positively identified, cloned, and their sequences reported. These are further subdivided based on their amino acid sequence homology, their ability to effect certain signal transduction mechanisms, and their known pharmacological properties. Ozawa, Kamiya and Tsuzuski, Prog. Neurobio., 54, 581 (1998). For instance, the Group I mGluR receptors, which include the mGlulR and mGlu5R, are known to activate phospholipase C (PLC) via Gaq-proteins thereby resulting in the increased hydrolysis of phosphoinositides and intracellular calcium mobilization. There are several compounds that are reported to activate the Group I mGlu receptors including DHPG, (R/S)-3,5-dihydroxyphenylglycine. Schoepp, Goldworthy, Johnson, Salhoff and Baker, J. Neurochem., 63, 769 (1994); Ito, et al., keurorep., 3, 1013 (1992). The Group II mGlu receptors consist of the two distinct receptors, mGluR2 and mGluR3 receptors. Both have been found to be negatively coupled to adenylate cyclase via activation of Gai-protein. These receptors can be activated by a selective compound such as 1S,2S,5R,6S-2 aminobicyclo[3.1.0]hexane-2,6-dicarboxylate. Monn, et al., J. Med. Chem., 40, 528 (1997); Schoepp, et al., Neuropharmacol., 36, 1 (1997). Similarly, the Group III mGlu receptors, including mGluR4, mGluR6, mGluR7 and mGluR8, are negatively coupled to adenylate cyclase via Gai and are potently activated by L-AP4 (L-(+)-2-amino-4-phosphonobutyric acid). Schoepp, Neurochem. Int., 24, 439 (1994).

It has become increasingly clear that there is a link between modulation of excitatory amino acid receptors, including the glutamatergic system, through changes in glutamate release or alteration in postsynaptic receptor activation, and a variety of neurological and psychiatric disorders. e.g. Monaghan, Bridges and Cotman, Ann. Rev. Pharmacol. Toxicol., 29, 365-402 (1989); Schoepp and Sacann, Neurobio. Aging, 15, 261-263 (1994); Meldrum and Garthwaite, Tr. Pharmacol. Sci., 11, 379-387 (1990). The medical consequences of such glutamate dysfunction makes the abatement of these neurological processes an important therapeutic goal.

### SUMMARY OF THE INVENTION

The present invention is directed to compounds which are potentiators of metabotropic glutamate receptors, including the mGluR2 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The present invention is directed to compounds of the formula I:

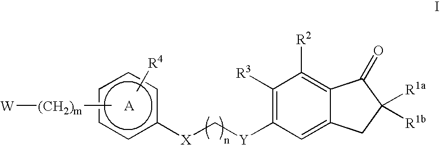

wherein:
A is phenyl or pyridyl;
W is selected from the group consisting of:

(1) tetrazolyl,

(2) CO₂H,

(3) NHSO₂C₁₋₆alkyl,

(4) NHSO₂-phenyl, wherein the phenyl is unsubstituted or substituted with C₁₋₆alkyl, and

(5) CONHCO—C₁₋₆alkyl;

X is selected from the group consisting of:

(1) —O—,

(2) —S—, and

(3) a bond;

(4) —O-phenyl-,

(5) —S-phenyl-, and

(6) -phenyl-;

Y is selected from the group consisting of:

(1) —O—,

(2) —NH(CO)—, and

(3) a bond;

R^{1a} and R^{1b} are independently selected from the group consisting of:

(1) hydrogen,

(2) C₁₋₆alkyl, which is unsubstituted or substituted with a substituent selected from:
(a) halogen,
(b) hydroxyl, and
(c) phenyl, wherein the phenyl is unsubstituted or substituted with 1-5 substituents independently selected from halogen, cyano, CF₃, hydroxyl, C₁₋₆alkyl, and OC₁₋₆alkyl,

(3) C₃₋₇cycloalkyl, which is unsubstituted or substituted with halogen, hydroxyl or phenyl, and

(4) phenyl, wherein the phenyl is unsubstituted or substituted with 1-5 substituents independently selected from halogen, hydroxyl, cyano, CF₃, C₁₋₆alkyl, and OC₁₋₆alkyl, wherein the C₁₋₆alkyl and OC₁₋₆alkyl are linear or branched and optionally substituted with 1-5 halogen;

R² is selected from the group consisting of:
(1) halogen,
(2) hydroxyl,
(3) OC₁₋₆alkyl, and
(4) C₁₋₆alkyl, which is unsubstituted or substituted with halogen, hydroxyl or phenyl;
   R³ is selected from the group consisting of:
(1) halogen, and
(2) C₁₋₆alkyl, which is unsubstituted or substituted with halogen, hydroxyl or phenyl;
   R⁴ may include multiple substituents and is independently selected from the group consisting of:
(1) hydrogen,
(2) halogen,
(3) C₁₋₆alkyl, and
(4) —O—C₁₋₆alkyl,
or R⁴ may be joined to the phenyl ring at an adjacent carbon to form a dihydrofuranyl ring;
   m is an integer selected from 0, 1, 2 and 3;
   n is an integer selected from 0, 1, 2, 3, 4, 5 and 6;
   and pharmaceutically acceptable salts thereof and individual diastereomers thereof.

An embodiment of the present invention includes compounds wherein A is phenyl.

An embodiment of the present invention includes compounds wherein A is pyridyl.

An embodiment of the present invention includes compounds wherein W is tetrazolyl.

An embodiment of the present invention includes compounds wherein W is CO₂H.

An embodiment of the present invention includes compounds wherein X is —O—.

An embodiment of the present invention includes compounds wherein Y is —O—.

An embodiment of the present invention includes compounds wherein X is a bond and Y is —O—.

An embodiment of the present invention includes compounds wherein X is a bond.

An embodiment of the present invention includes compounds wherein X is —O-phenyl-.

An embodiment of the present invention includes compounds wherein X is —O—1,3-phenyl-.

An embodiment of the present invention includes compounds wherein X is -phenyl-.

An embodiment of the present invention includes compounds wherein X is -1,3-phenyl-.

An embodiment of the present invention includes compounds wherein R^{1a} is C₁₋₆alkyl.

An embodiment of the present invention includes compounds wherein R^{1a} is C₅₋₆cycloalkyl.

An embodiment of the present invention includes compounds wherein R^{1a} is CH₃.

An embodiment of the present invention includes compounds wherein R^{1a} is CH₂CH₂CH₃.

An embodiment of the present invention includes compounds wherein R^{1a} is CH₂CH₂CH₂CH₃.

An embodiment of the present invention includes compounds wherein R^{1a} is cyclopentyl.

An embodiment of the present invention includes compounds wherein R^{1a} is CH₂-cyclopentyl.

An embodiment of the present invention includes compounds wherein R^{1a} is phenyl.

An embodiment of the present invention includes compounds wherein R^{1b} is hydrogen.

An embodiment of the present invention includes compounds wherein R^{1b} is C₁₋₆alkyl.

An embodiment of the present invention includes compounds wherein R^{1b} is CH₃.

An embodiment of the present invention includes compounds wherein R^{1b} is CH₂CH₂CH₂CH₃.

An embodiment of the present invention includes compounds wherein R^{1a} is C₅₋₆cycloalkyl and R^{1b} is C₁₋₆alkyl.

An embodiment of the present invention includes compounds wherein R^{1a} is C₅₋₆cycloalkyl and R^{1b} is hydrogen.

An embodiment of the present invention includes compounds wherein R^{1a} is cyclopentyl and R^{1b} is hydrogen.

An embodiment of the present invention includes compounds wherein R^{1a} is cyclopentyl and R^{1b} is CH₃.

An embodiment of the present invention includes compounds wherein R^{1a} is CH₂-cyclopentyl and R^{1b} is CH₃.

An embodiment of the present invention includes compounds wherein R^{1a} is CH₂-Cyclopentyl and R^{1b} is CH₂CH₂CH₂CH₃.

An embodiment of the present invention includes compounds wherein R² is chloro.

An embodiment of the present invention includes compounds wherein R³ is chloro.

An embodiment of the present invention includes compounds wherein R² is chloro and R³ is chloro.

An embodiment of the present invention includes compounds wherein R⁴ is hydrogen or bromo.

An embodiment of the present invention includes compounds wherein R⁴ is hydrogen.

An embodiment of the present invention includes compounds wherein m is 0.

An embodiment of the present invention includes compounds wherein m is 1.

An embodiment of the present invention includes compounds wherein n is 0.

An embodiment of the present invention includes compounds wherein n is 1.

An embodiment of the present invention includes compounds wherein n is 2.

An embodiment of the present invention includes compounds wherein n is 3.

An embodiment of the present invention includes compounds wherein n is 4.

Specific embodiments of the present invention include a compound which is selected from the group consisting of:
6,7-Dichloro-2-cyclopentyl-2-methyl-5-{3-[4-(2H-tetrazol-5-yl)-phenoxy]-propoxy}-indan-1-one;
6,7-Dichloro-2-cyclopentyl-2-methyl-5-{2-[4-(2H-tetrazol-5-yl)-phenoxy]-ethoxy}-indan-1-one;
6,7-Dichloro-2-cyclopentyl-2-methyl-5-[4-(2H-tetrazol-5-yl)-benzyloxy]-1-indan-e-one;
6,7-Dichloro-2-cyclopentyl-2-methyl-5-[5-(2H-tetrazol-5-yl)-pentyloxy]-1-indan-1-one;
6,7-Dichloro-2-cyclopentyl-5-[4-(2H-tetrazol-5-yl)-benzyloxy]-indan-1-one;
6,7-Dichloro-2-propyl-5-[4-(2H-tetrazol-5-yl)-benzyloxy]-indan-1-one;
6,7-Dichloro-2-cyclopentyl-2-methyl-5-[4-(2H-tetrazol-5-yl)-butoxy]-indan-1-one;
6,7-Dichloro-2-isopropyl-5-[4-(2H-tetrazol-5-yl)-benzyloxy]-indan-1-one;
6,7-Dichloro-2-cyclopentyl-2-methyl-5-[4-(2H-tetrazol-5-yl)-phenylethynyl]-indan-1-one;
6,7-Dichloro-2-cyclopentyl-2-methyl-5-{2-[4-(2H-tetrazol-5-yl)-phenyl]-ethyl}-indan-1-one;
6,7-Dichloro-2,2-dimethyl-5-[4-(2H-tetrazol-5-yl)-benzyloxy]-indan-1-one;
2-(6,7-Dichloro-2-cyclopentyl-2-methyl-1-oxo-indan-5-yloxy)-N-[4-(1H-tetrazol-5-yl)-phenyl]-acetamide;
6,7-Dichloro-2-cyclopentylmethyl-2-methyl-5-[4-(1H-tetrazol-5-yl)-benzyloxy]-indan-1-one;
6,7-Dichloro-2-cyclopentyl-2-methyl-5-[3-(1H-tetrazol-5-yl)-benzyloxy]-indan-1-one;
6,7-Dichloro-2-cyclopentyl-2-methyl-5-[3-(1H-tetrazol-5-yl)-propoxy]-indan-1-one;
4-(6,7-Dichloro-2-cyclopentyl-2-methyl-1-oxo-indan-5-yloxymethyl)-benzoic acid;
6,7-Dichloro-2-methyl-2-phenyl-5-[4-(1H-tetrazol-5-yl)-benzyloxy]-indan-1-one;
2-Butyl-6,7-dichloro-2-cyclopentyl-5-[4-(1H-tetrazol-5-yl)-benzyloxy]-indan-1-one;
N-[4-(6,7-Dichloro-2-cyclopentyl-2-methyl-1-oxo-indan-5-yloxymethyl)-benzoyl]-methanesulfonamide;
N-[4-(6,7-Dichloro-2-cyclopentyl-2-methyl-1-oxo-indan-5-yloxymethyl)-benzoyl]-4-methyl-benzenesulfonamide;
6,7-Dichloro-2-cyclopentyl-2-methyl-5-[4-(1H-tetrazol-5-yl)-phenyl]-indan-1-one;
3,5-dibromo-4-{[(6,7-dichloro-2-cyclopentyl-2-methyl-1-oxo-2,3-dihydro-1H-inden-5-yl)oxy]methyl}-N-(methylsulfonyl)benzamide;
N-acetyl-4-{[6,7-dichloro-2-cyclopentyl-2-methyl-1-oxo-2,3-dihydro-1H-inden-5-yl)oxy]methyl}benzamide;
6,7-dichloro-2-cyclopentyl-2-methyl-5-{[5-(1H-tetrazol-5-yl)pyridin-2-yl]methoxy}indan-1-one;
6,7-dichloro-2-cyclopentyl-2-methyl-5-{4-[4-(2H-tetrazol-5-yl)phenoxy]butoxy} indan-1-one;
6,7-dichloro-2-cyclopentyl-2-methyl-5-{4-[3-(2H-tetrazol-5-yl)phenoxy]butoxy}indan-1-one;
3′-{[(2-Cyclopentyl-6,7-dimethyl-1-oxo-2,3-dihydro-1H-inden-5-yl)oxy]methyl}biphenyl-3-carboxylic acid;
5-(3-{[(2-Cyclopentyl-6,7-dimethyl-1-oxo-2,3-dihydro-1H-inden-5-yl)oxy]methyl}phenyl)nicotinic acid
2-Cyclopentyl-6,7-dimethyl-5-({3-[5-(1H-tetrazol-5-yl)pyridin-3-yl]benzyl}oxy)indan-1-one;
6,7-dichloro-2-cyclopentyl-2-methyl-5-({3-[4-(2H-tetrazol-5-yl)phenoxy]benzyl)oxy}indan-1-one

### 6-chloro-2-cyclopentyl-2-methyl-5-({3-[4-(2H-tetrazol-5-yl)phenoxy]benzyl}oxy)indan-1-one;

2-cyclopentyl-6,7-dimethyl-5-{[3′-(2H-tetrazol-5-yl)biphenyl-3-yl]methoxy}indan-1-one;
3′-{[(2-Cyclopentyl-6,7-dimethyl-1-oxo-2,3-dihydro-1H-inden-5-yl)oxy]methyl}biphenyl-4-carboxylic acid;
3′-{[(6,7-Dichloro-2-cyclopentyl-2-methyl-1-oxo-2,3-dihydro-1H-inden-5-yl)oxy]methyl}biphenyl-3-carboxylic acid;
3′-{[(2-cyclopentyl-2,6,7-trimethyl-1-oxo-2,3-dihydro-1H-inden-5-yl)oxy]methyl}biphenyl-4-carboxylic acid;
3′-{[(2-cyclopentyl-2,6,7-trimethyl-1-oxo-2,3-dihydro-1H-inden-5-yl)oxy]methyl}biphenyl-3-carboxylic acid;
2-cyclopentyl-6,7-dimethyl-5-{[4′-(2H-tetrazol-5-yl)biphenyl-3-yl]methoxy}indan-1-one;
3-(4-{4-[(6,7-dichloro-2-cyclopentyl-2-methyl-1-oxo-2,3-dihydro-1H-inden-5-yl)oxy]butoxy}phenyl)propanoic acid;
3′-{[(6,7-Dichloro-2-cyclopentyl-2-methyl-1-oxo-2,3-dihydro-1H-inden-5-yl)oxy]methyl}biphenyl-4-carboxylic acid;
5-(3-{[(2-Cyclopentyl-6,7-dimethyl-1-oxo-2,3-dihydro-1H-inden-5-yl)oxy]methyl}phenyl)pyridine-2-carboxylic acid;
4-{3-[(2-cyclopentyl-2,6,7-trimethyl-1-oxo-2,3-dihydro-1H-inden-5-yl)oxy]methyl}phenoxy)benzoic acid;
3′-{[(2-cyclopentyl-6,7-dimethyl-1-oxo-2,3-dihydro-1H-inden-5-yl)oxy]methyl}-N-(methylsulfonyl)biphenyl-3-carboxamide;
3′-{[(2-Cyclopentyl-6,7-dimethyl-1-oxo-2,3-dihydro-1H-inden-5-yl)oxy]methyl}-2-methylbiphenyl-3-carboxylic acid;
3′-{[(2-Cyclopentyl-6,7-dimethyl-1-oxo-2,3-dihydro-1H-inden-5-yl)oxy]methyl}-3-methylbiphenyl-4-carboxylic acid;
3′-{[(2-Cyclopentyl-6,7-dimethyl-1-oxo-2,3-dihydro-1H-inden-5-yl)oxy]methyl}-2-methylbiphenyl-4-carboxylic acid;
4-Chloro-3′-{[(2-cyclopentyl-6,7-dimethyl-1-oxo-2,3-dihydro-1H-inden-5-yl)oxy]methyl}biphenyl-3-carboxylic acid;
3′-{[(2-Cyclopentyl-6,7-dimethyl-1-oxo-2,3-dihydro-1H-inden-5-yl)oxy]methyl}-6-methylbiphenyl-3-carboxylic acid;
3′-{[(6,7-Dichloro-2-cyclopentyl-1-oxo-2,3-dihydro-1H-inden-5-yl)oxy]methyl}biphenyl-4-carboxylic acid;
3′-{[(6,7-Dichloro-2-isopropyl-1-oxo-2,3-dihydro-1H-inden-5-yl)oxy]methyl}biphenyl-4-carboxylic acid;
3′-{[(6,7-Dichloro-1-oxo-2-propyl-2,3-dihydro-1H-inden-5-yl)oxy]methyl}biphenyl-4-carboxylic acid;
5-({2-chloro-5-[4-(2H-tetrazol-5-yl)phenoxy]benzyl}oxy)-2-cyclopentyl-6,7-dimethylindan-1-one;
4-(3-{([(2-cyclopentyl-6,7-dimethyl-1-oxo-2,3-dihydro-1H-inden-5-yl)oxy]methyl}phenoxy)benzoic acid;
4-(3-{[(6,7-dichloro-2-cyclopentyl-2-methyl-1-oxo-2,3-dihydro-1H-inden-5-yl)oxy]methyl}phenoxy)benzoic acid;
3′-{[(6,7-Dichloro-2,2-dimethyl-1-oxo-2,3-dihydro-1H-inden-5-yl)oxy]methyl}biphenyl-4-carboxylic acid;
3′-{[(6,7-Dichloro-2-methyl-1-oxo-2-phenyl-2,3-dihydro-1H-inden-5-yl)oxy]methyl}biphenyl-4-carboxylic acid;
3′-{[(2-Butyl-6,7-dichloro-2-cyclopentyl-1-oxo-2,3-dihydro-1H-inden-5-yl)oxy]methyl}biphenyl-4-carboxylic acid;
3′-{([6,7-Dichloro-2-(cyclopentylmethyl)-2-methyl-1-oxo-2,3-dihydro-1H-inden-5-yl]oxy}methyl)biphenyl-4-carboxylic acid;
3′-{[(7-Chloro-2-cyclopentyl-6-methyl-1-oxo-2,3-dihydro-1H-inden-5-yl)oxy]methyl}biphenyl-4-carboxylic acid;
3′-{[(2-Cyclopentyl-6,7-dimethyl-1-oxo-2,3-dihydro-1H-inden-5-yl)oxy]methyl}-6-fluorobiphenyl-3-carboxylic acid
3′-{[(2-Cyclopentyl-6,7-dimethyl-1-oxo-2,3-dihydro-1H-inden-5-yl)oxy]methyl}-2-fluorobiphenyl-4-carboxylic acid;
3′-{[(2-Cyclopentyl-6,7-dimethyl-1-oxo-2,3-dihydro-1H-inden-5-yl)oxy]methyl}-6-methoxybiphenyl-3-carboxylic acid;
3′-{[(2-Cyclopentyl-6,7-dimethyl-1-oxo-2,3-dihydro-1H-inden-5-yl)oxy]methyl}-2,6-dimethoxybiphenyl-4-carboxylic acid;
3-Chloro-3′-{[(2-cyclopentyl-6,7-dimethyl-1-oxo-2,3-dihydro-1H-inden-5-yl)oxy]methyl}biphenyl-4-carboxylic acid;
4-Chloro-3′-{[(6,7-dichloro-2-cyclopentyl-1-oxo-2,3-dihydro-1H-inden-5-yl)oxy]methyl}biphenyl-3-carboxylic acid;
4-Chloro-3′-{([(6,7-dichloro-2-cyclopentyl-2-methyl-1-oxo-2,3-dihydro-1H-inden-5-yl)oxy]methyl}biphenyl-3-carboxylic acid;
3′-{[(2-Cyclopentyl-6,7-dimethyl-1-oxo-2,3-dihydro-1H-inden-5-yl)oxy]methyl}-5-fluorobiphenyl-3-carboxylic acid;
3′-{[(6,7-Dichloro-2-cyclopentyl-2-methyl-1-oxo-2,3-dihydro-1H-inden-5-yl)oxy]methyl}-5-fluorobiphenyl-3-carboxylic acid;
3′-{[(2-Cyclopentyl-6,7-dimethyl-1-oxo-2,3-dihydro-1H-inden-5-yl)oxy]methyl}-4-hydroxybiphenyl-3-carboxylic acid;
3′-{[(2-Cyclopentyl-6,7-dimethyl-1-oxo-2,3-dihydro-1H-inden-5-yl)oxy]methyl}-4-methoxybiphenyl-3-carboxylic acid;
5-(3-{[(2-Cyclopentyl-6,7-dimethyl-1-oxo-2,3-dihydro-1H-inden-5-yl)oxy]methyl}phenyl)-2,3-dihydro-1-benzofuran-7-carboxylic acid;
3′-{[(7-Chloro-2-cyclopentyl-6-methyl-1-oxo-2,3-dihydro-1H-inden-5-yl)oxy]methyl}biphenyl-3-carboxylic acid;
3′-{[(7-Chloro-2-cyclopentyl-6-methyl-1-oxo-2,3-dihydro-1H-inden-5-yl)oxy]methyl}-5-fluorobiphenyl-3-carboxylic acid;
4-Chloro-3′-{[7-chloro-2-cyclopentyl-6-methyl-1-oxo-2,3-dihydro-1H-inden-5-yl)oxy]methyl}biphenyl-3-carboxylic acid;
3′-{[(2-Cyclopentyl-6,7-dimethyl-1-oxo-2,3-dihydro-1H-inden-5-yl)oxy]methyl}-4-fluorobiphenyl-3-carboxylic acid;
3′-{[(2-Cyclopentyl-6,7-dimethyl-1-oxo-2,3-dihydro-1H-inden-5-yl)oxy]methyl}biphenyl-3,4-dicarboxylic acid;
6,7-Dichloro-2-cyclopentyl-2-methyl-5-{[3′-(2H-tetrazol-5-yl)biphenyl-3-yl]methoxy}indan-1-one;
   and pharmaceutically acceptable salts thereof.

The compounds of the present invention are potentiators of metabotropic glutamate (mGluR) receptor function, in particular they are potentiators of mGluR2 receptors. That is, the compounds of the present invention do not appear to bind at the glutamate recognition site on the mGluR receptor, but in the presence of glutamate or a glutamate agonist, the compounds of the present invention increase mGluR receptor response. The present potentiators are expected to have their effect at mGluR receptors by virtue of their ability to increase the response of such receptors to glutamate or glutamate agonists, enhancing the function of the receptors. It is recognized that the compounds of the present invention would be expected to increase the effectiveness of glutamate and glutamate agonists of the mGluR2 receptor. Thus, the potentiators of the present invention are expected to be useful in the treatment of various neurological and psychiatric disorders associated with glutamate dysfunction described to be treated herein and others that can be treated by such potentiators as are appreciated by those skilled in the art.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. The present invention is meant to comprehend all such isomeric forms of these compounds. Formula I shows the structure of the class of compounds without preferred stereochemistry.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As appreciated by those of skill in the art, halo or halogen as used herein are intended to include fluoro, chloro, bromo and iodo. Similarly, C₁₋₆, as in C₁₋₆alkyl is defined to identify the group as having 1, 2, 3, 4, 5 or 6 carbons in a linear or branched arrangement, such that C₁₋₈alkyl specifically includes methyl, ethyl, n-propyl, iso-propyl, n-butyl, iso-butyl, tert-butyl, pentyl, and hexyl. A group which is designated as being independently substituted with substituents may be independently substituted with multiple numbers of such substituents.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of Formula I are meant to also include the pharmaceutically acceptable salts.

Exemplifying the invention is the use of the compounds disclosed in the Examples and herein. Specific compounds within the present invention include a compound which selected from the group consisting of the compounds disclosed in the following Examples and pharmaceutically acceptable salts thereof and individual diastereomers thereof.

The subject compounds are useful in a method of potentiating metabotorpic glutamate receptor activity in a patient such as a mammal in need of such inhibition comprising the administration of an effective amount of the compound. The present invention is directed to the use of the compounds disclosed herein as potentiators of metabotorpic glutamate receptor activity. In addition to primates, especially humans, a variety of other mammals can be treated according to the method of the present invention.

The present invention is further directed to a method for the manufacture of a medicament for potentiating metabotorpic glutamate receptor activity in humans and animals comprising combining a compound of the present invention with a pharmaceutical carrier or diluent.

The subject treated in the present methods is generally a mammal, preferably a human being, male or female, in whom potentiation of metabotorpic glutamate receptor activity is desired. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as inhibitors of metabotropic glutamate receptor activity, in particular mGluR2 activity, may be demonstrated by methodology known in the art. Inhibition constants are determined as follows. The compounds of the present invention were tested in a [³⁵S]-GTPγS assay. The stimulation of [³⁵S]-GTPγS binding is a common functional assay to monitor Gαi-coupled receptor in native and recombinant receptor membrane preparation. Membrane from cells stably expressing hmGlu2 CHO-K1 (50 μg) were incubated in a 96 well plate for 1 hour in the presence of GTPγS³⁵ (0.05 nM), GDP (5 μM) and compounds. The reaction was stopped by rapid filtration over Unifilter GF/B plate (Packard, Bioscience, Meriden Conn.) using a 96-well cell harvester (Brandel Gaithersburg, Md.). The filter plates were counted using Topcount counter (Packard, Bioscience, Meriden Conn., USA). When compounds were evaluated as potentiator they were tested in the presence of glutamate (1 μM). The activation (agonist) or the potentiation of glutamate (potentiator) curves were fitted with a four parameters logistic equation giving EC₅₀ and Hill coefficient using the iterative non linear curve fitting software GraphPad (San Diego Calif., USA).

In particular, the compounds of the following examples had activity in potentiating the mGluR2 receptor in the aforementioned assays, generally with an EC₅₀ of less than about 10 μM. Preferred compounds within the present invention had activity in potentiating the mGluR2 receptor in the aforementioned assays with an EC₅₀ of less than about 1 μM. Such a result is indicative of the intrinsic activity of the compounds in use as potentiators of mGluR2 receptor activity.

Metabotropic glutamate receptors including the mGluR2 receptor have been implicated in a wide range of biological functions. This has suggested a potential role for these receptors in a variety of disease processes in humans or other species.

The compounds of the present invention have utility in treating, preventing, ameliorating, controlling or reducing the risk of a variety of neurological and psychiatric disorders associated with glutamate dysfunction, including one or more of the following conditions or diseases: acute neurological and psychiatric disorders such as cerebral deficits subsequent to cardiac bypass surgery and grafting, stroke, cerebral ischemia, spinal cord trauma, head trauma, perinatal hypoxia, cardiac arrest, hypoglycemic neuronal damage, dementia (including AIDS-induced dementia), Alzheimer's disease, Huntington's Chorea, amyotrophic lateral sclerosis, ocular damage, retinopathy, cognitive disorders, idiopathic and drug-induced Parkinson's disease, muscular spasms and disorders associated with muscular spasticity including tremors, epilepsy, convulsions, migraine (including migraine headache), urinary incontinence, substance tolerance, substance withdrawal (including, substances such as opiates, nicotine, tobacco products, alcohol, benzodiazepines, cocaine, sedatives, hypnotics, etc.), psychosis, schizophrenia, anxiety (including generalized anxiety disorder, panic disorder, and obsessive compulsive disorder), mood disorders (including depression, mania, bipolar disorders), trigeminal neuralgia, hearing loss, tinnitus, macular degeneration of the eye, emesis, brain edema, pain (including acute and chronic pain states, severe pain, intractable pain, neuropathic pain, and post-traumatic pain), tardive dyskinesia, sleep disorders (including narcolepsy), attention deficit/hyperactivity disorder, and conduct disorder.

Of the disorders above, the treatment of migraine, anxiety, schizophrenia, and epilepsy are of particular importance. In a preferred embodiment the present invention provides a method for treating migraine, comprising:

administering to a patient in need thereof an effective amount of a compound of formula I. In another preferred embodiment the present invention provides a method for preventing or treating anxiety, comprising: administering to a patient in need thereof an effective amount of a compound of formula I. Particularly preferred anxiety disorders are generalized anxiety disorder, panic disorder, and obsessive compulsive disorder. In another preferred embodiment the present invention provides a method for treating schizophrenia, comprising: administering to a patient in need thereof an effective amount of a compound of formula I. In yet another preferred embodiment the present invention provides a method for treating epilepsy, comprising: administering to a patient in need thereof an effective amount of a compound of formula I.

Of the neurological and psychiatric disorders associated with glutamate dysfunction which are treated according to the present invention, the treatment of migraine, anxiety, schizophrenia, and epilepsy are particularly preferred. Particularly preferred anxiety disorders are generalized anxiety disorder, panic disorder, and obsessive compulsive disorder.

Thus, in a preferred embodiment the present invention provides a method for treating migraine, comprising: administering to a patient in need thereof an effective amount of a compound of formula I or a pharmaceutical composition thereof. In one of the available sources of diagnostic tools, Dorland's Medical Dictionary (23'd Ed., 1982, W. B. Saunders Company, Philadelphia, Pa.), migraine is defined as a symptom complex of periodic headaches, usually temporal and unilateral, often with irritability, nausea, vomiting, constipation or diarrhea, and photophobia. As used herein the term “migraine” includes these periodic headaches, both temporal and unilateral, the associated irritability, nausea, vomiting, constipation or diarrhea, photophobia, and other associated symptoms. The skilled artisan will recognize that there are alternative nomenclatures, nosologies, and classification systems for neurological and psychiatric disorders, including migraine, and that these systems evolve with medical scientific progress.

In another preferred embodiment the present invention provides a method for treating anxiety, comprising: administering to a patient in need thereof an effective amount of a compound of formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including anxiety and related disorders. These include: panic disorder with or without agoraphobia, agoraphobia without history of panic disorder, specific phobia, social phobia, obsessive-compulsive disorder, post-traumatic stress disorder, acute stress disorder, generalized anxiety disorder, anxiety disorder due to a general medical condition, substance-induced anxiety disorder and anxiety disorder not otherwise specified. As used herein the term “anxiety” includes treatment of those anxiety disorders and related disorder as described in the DSM-IV. The skilled artisan will recognize that there are alternative nomenclatures, nosologies, and classification systems for neurological and psychiatric disorders, and particular anxiety, and that these systems evolve with medical scientific progress. Thus, the term “anxiety” is intended to include like disorders that are described in other diagnostic sources.

In another preferred embodiment the present invention provides a method for treating depression, comprising: administering to a patient in need thereof an effective amount of a compound of formula I or a pharmaceutical composition thereof. At present, the fourth edition of the Diagnostic and Statistical Manual of Mental Disorders (DSM-IV) (1994, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder. As used herein the term “depression” includes treatment of those depression disorders and related disorder as described in the DSM-IV.

In another preferred embodiment the present invention provides a method for treating epilepsy, comprising: administering to a patient in need thereof an effective amount of a compound of formula I or a pharmaceutical composition thereof. At present, there are several types and subtypes of seizures associated with epilepsy, including idiopathic, symptomatic, and cryptogenic. These epileptic seizures can be focal (partial) or generalized. They can also be simple or complex. Epilepsy is described in the art, such as Epilepsy: A comprehensive textbook. Ed. by Jerome Engel, Jr. and Timothy A. Pedley. (Lippincott-Raven, Philadelphia, 1997). At present, the International Classification of Diseases, Ninth Revision, (ICD-9) provides a diagnostic tool including epilepsy and related disorders. These include: generalized nonconvulsive epilepsy, generalized convulsive epilepsy, petit mal status epilepticus, grand mal status epilepticus, partial epilepsy with impairment of consciousness, partial epilepsy without impairment of consciousness, infantile spasms, epilepsy partialis continua, other forms of epilepsy, epilepsy, unspecified, NOS. As used herein the term “epilepsy” includes these all types and subtypes. The skilled artisan will recognize that there are alternative nomenclatures, nosologies, and classification systems for neurological and psychiatric disorders, including epilepsy, and that these systems evolve with medical scientific progress.

The subject compounds are further useful in a method for the prevention, treatment, control, amelioration, or reducation of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The term “potentiated amount” refers to an amount of an mGluR agonist, that is, the dosage of agonist which is effective in treating the neurological and psychiatric disorders described herein when administered in combination with an effective amount of a compound of the present invention. A potentiated amount is expected to be less than the amount that is required to provided the same effect when the mGluR agonist is administered without an effective amount of a compound of the present invention.

A potentiated amount can be readily determined by the attending diagnostician, as one skilled in the art, by the use of conventional techniques and by observing results obtained under analogous circumstances. In determining a potentiated amount, the dose of an mGluR agonist to be administered in combination with a compound of formula I, a number of factors are considered by the attending diagnostician, including, but not limited to: the mGluR agonist selected to be administered, including its potency and selectivity; the compound of formula I to be coadministered; the species of mammal; its size, age, and general health; the specific disorder involved; the degree of involvement or the severity of the disorder; the response of the individual patient; the modes of administration; the bioavailability characteristics of the preparations administered; the dose regimens selected; the use of other concomitant medication; and other relevant circumstances.

A potentiated amount of an mGluR agonist to be administered in combination with an effective amount of a compound of formula I is expected to vary from about 0.1 milligram per kilogram of body weight per day (mg/kg/day) to about 100 mg/kg/day and is expected to be less than the amount that is required to provided the same effect when administered without an effective amount of a compound of formula I. Preferred amounts of a co-administered mGlu agonist are able to be determined by one skilled in the art.

The compounds of the present invention may be used in combination with one or more other drugs in the treatment, prevention, control, amelioration, or reduction of risk of diseases or conditions for which compounds of Formula I or the other drugs may have utility, where the combination of the drugs together are safer or more effective than either drug alone. Such other drug(s) may be administered, by a route and in an amount commonly used therefor, contemporaneously or sequentially with a compound of Formula I. When a compound of Formula I is used contemporaneously with one or more other drugs, a pharmaceutical composition in unit dosage form containing such other drugs and the compound of Formula I is preferred. However, the combination therapy may also includes therapies in which the compound of Formula I and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of Formula I.

The above combinations include combinations of a compound of the present invention not only with one other active compound, but also with two or more other active compounds.

Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. When a compound of the present invention is used contemporaneously with one or more other drugs, a pharmaceutical composition containing such other drugs in addition to the compound of the present invention is preferred. Accordingly, the pharmaceutical compositions of the present invention include those that also contain one or more other active ingredients, in addition to a compound of the present invention.

The weight ratio of the compound of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, preferably about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys. etc. the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The pharmaceutical composition and method of the present invention may further comprise other therapeutically active compounds as noted herein which are usually applied in the treatment of the above mentioned pathological conditions.

In the treatment, prevention, control, amelioration, or reduction of risk of conditions which require potentiation of metabotorpic glutamate receptor activity an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0. 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

When treating, preventing, controlling, ameliorating, or reducing the risk of neurological and psychiatric disorders associated with glutamate dysfunction or other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 1000 milligrams, preferably from about 1 milligrams to about 50 milligrams. In the case of a 70 kg adult human, the total daily dose will generally be from about 7 milligrams to about 350 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials are made according to procedures known in the art or as illustrated herein.

The compounds of the present invention can be prepared in a variety of fashions.

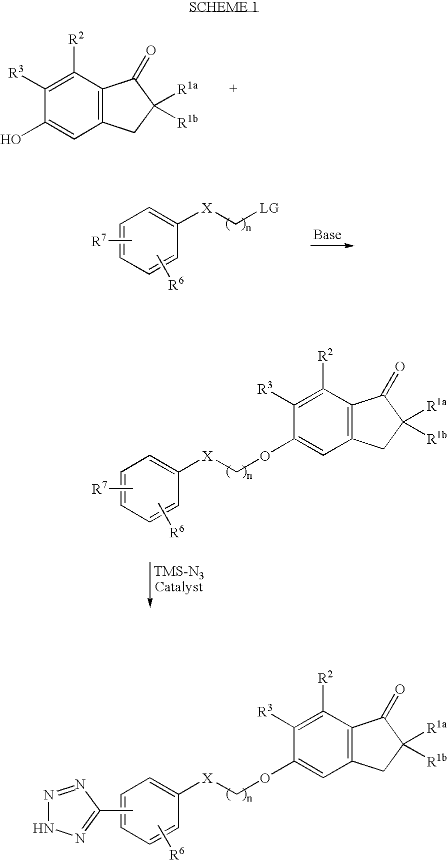

An appropriately substituted indanone containing a tetrazole can be prepared via an alkylation followed by conversion of the nitrile derivative to the tetrazole as outlined in Scheme 1. For the alkylation, an indanone (prepared using techniques well known in the art and described in the literature, see Woltersdorf et. al., J. Med. Chem., 1977, 20, 1400 and references therein) is reacted with linkers with a suitable leaving group (LG=halide, triflate, tosylate, mesylate and the like) (wherein R6 is selected from R4). This reaction is carried out in the presence of a base (potassium carbonate, sodium hydroxide, and the like) in a suitable solvent (acetone, tetrahydrofuran, dimethoxyethane, etc.). The reaction is generally run at ambient temperature to 45° C. for a period of 4 to 24 hours. The product from the reaction can be isolated and purified employing standard techniques such as solvent extraction, chromatography, crystallization, distillation and the like. For tetrazole formation. When R7=CN, the nitrile containing compound is reacted with trimethylsilyl azide in the presence of a catalyst such as dibutyltin oxide in a suitable solvent (benzene, toluene, mesitylene and the like) at an appropriate temperature, usually 110° C. for a period of 8-16 hours. The product from the reaction can be isolated and purified employing standard techniques such as solvent extraction, chromatography, crystallization, distillation and the like.

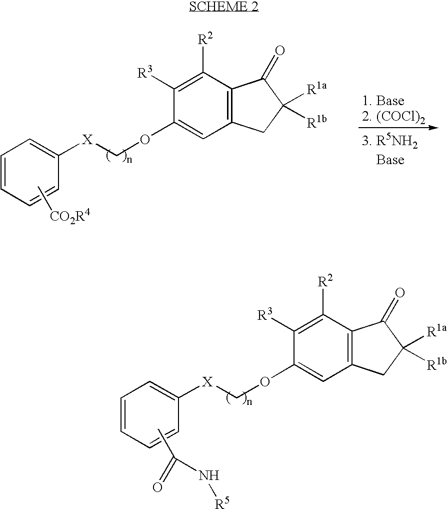

The alkylated compounds (when R7=ester) can also be converted into carboxylic acids, amides, sulfonamides and imides as shown in scheme 2. Thus, in scheme 2, the ester derivative is first hydrolyzed in the presence of a suitable base (lithium hydroxide, sodium hydroxide and the like) in a solvent such as water/dioxane of water/tetrahydrofuran to provide the corresponding carboxylic acid. The reaction is generally run at ambient temperature for a period of 1-16 hours. The carboxylic acid can be further reacted by first converting it to the acid chloride via reaction with oxalyl chloride (or other reagents such as thionyl chloride) in a suitable solvent such as dichloromethane. This acid chloride can then be further reacted with a variety of nitrogen compounds such as amides and sulfonamides in the presence of a base such as sodium hydride or lithium diisopropyl amide in a suitable solvent such as tetrahydrofuran to give the desired compound. The reaction is generally run at temperatures from −78 to 0° C. for a period of 4-12 hours. The product from the reaction can be isolated and purified employing standard techniques such as solvent extraction, chromatography, crystallization, distillation and the like.

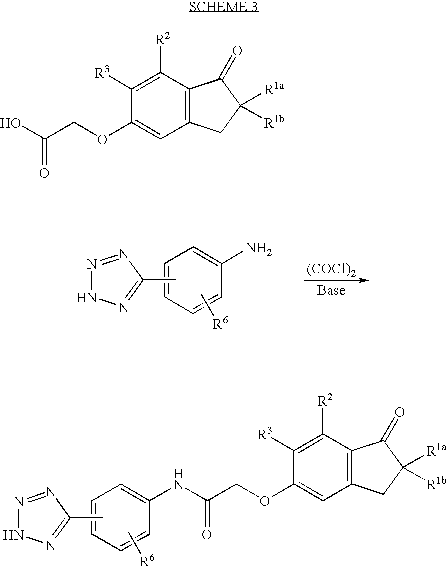

Compounds containing an amide linkage can be prepared as outlined in scheme 3. As illustrated, an indanone containing a carboxylic acid (prepared using techniques well known in the art and described in the literature, see Woltersdorf et. al., J. Med. Chem., 1977, 20, 1400 and references therein) is converted into the acid chloride. This reaction is performed via reaction with oxalyl chloride (or other reagents such as thionyl chloride) in a suitable solvent such as dichloromethane. This acid chloride can then be further reacted with an appropriate aniline (wherein R6 is selected from R4) in the presence of a base such as sodium hydride or lithium diisopropyl amide in a suitable solvent such as tetrahydrofuran to give the desired compound. The product from the reaction can be isolated and purified employing standard techniques such as solvent extraction, chromatography, crystallization, distillation and the like.

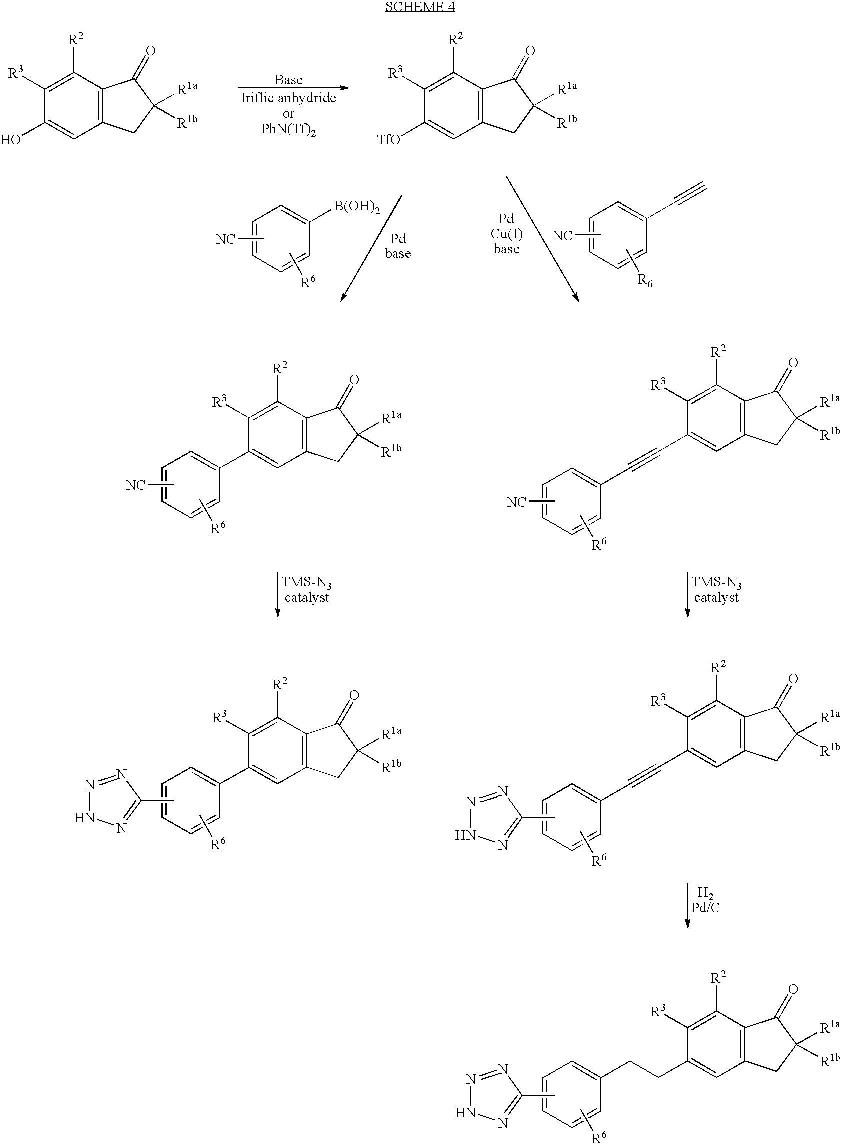

Indanones containing all carbon linkages can be prepared as outlined in scheme 4, wherein the indanone is first converted into the corresponding triflate and then subjected to palladium catalyzed couplings with either an acetylene or a boronic acid using techniques well known in the art (wherein R6 is selected from R4). These reactions are normally carried out in the presence of a base, in a suitable solvent such as dimethylformamide or dioxane. The product from the reaction can be isolated and purified employing standard techniques such as solvent extraction, chromatography, crystallization, distillation and the like. The products from the palladium catalyzed couplings are then transformed into tetrazoles as outlined in scheme 1. The acetylene derivative can be reduced further using hydrogen gas and a catalyst such as palladium on carbon following techniques well known in the art.

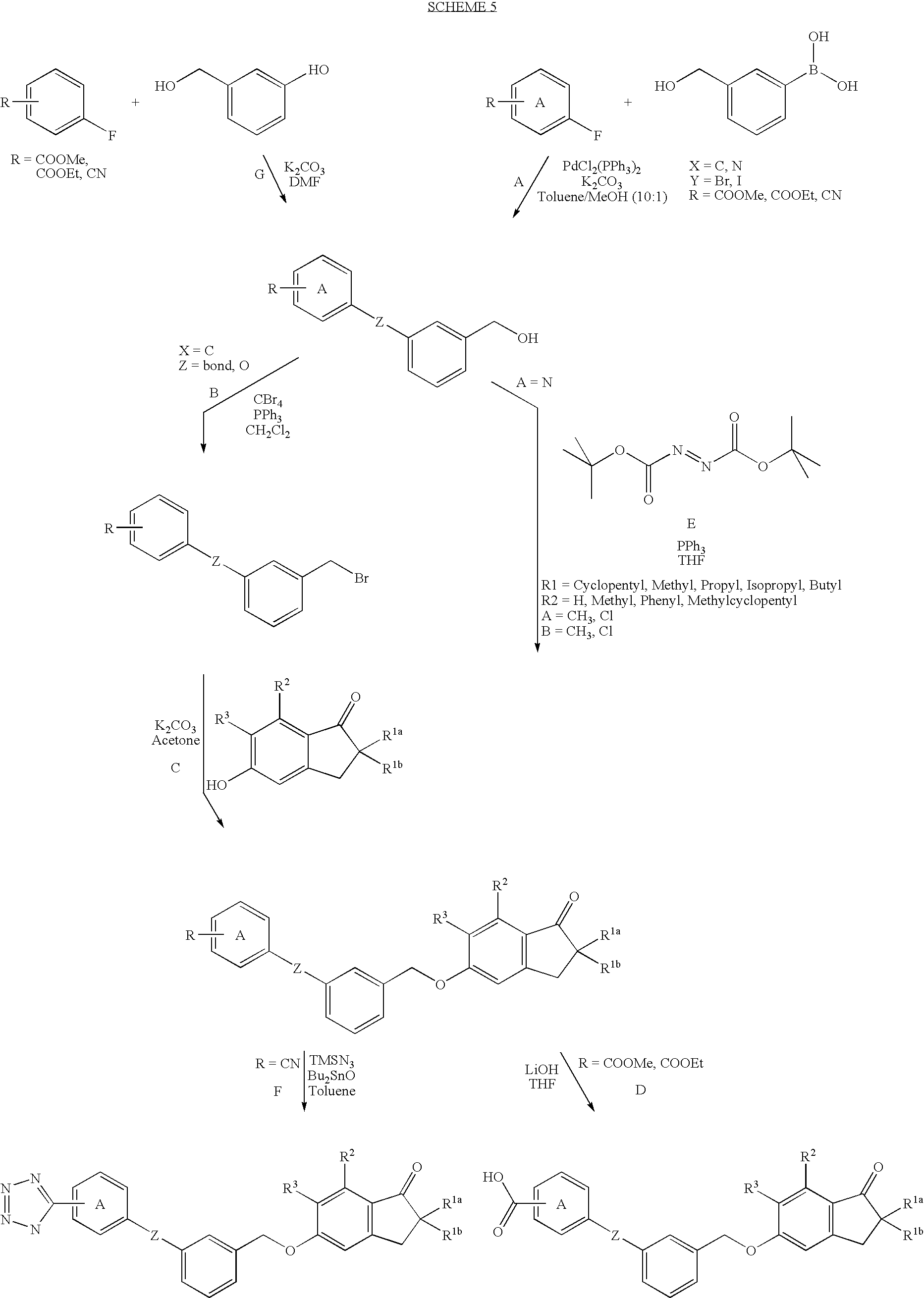

A variety of indanones containing aryl linkers, either carbon or oxygen linked, can be prepared as outlined in scheme 5. Precursors can be prepared as outlined in steps A and G in scheme 5 using either aromatic substitutions or transition metal catalyzed cross couplings following techniques well known in the art. The indanone is the alkylated via the corresponding benzyl bromide using typical bases (steps B/C) or subjected to a Mitsunobu type reaction using reagents such as ditertbutylazodicarboxylate (step E). The desired indanone compounds can then be accessed by either ester hydrolysis (step D) or tetrazole formation (step F), both of which have been outlined above.

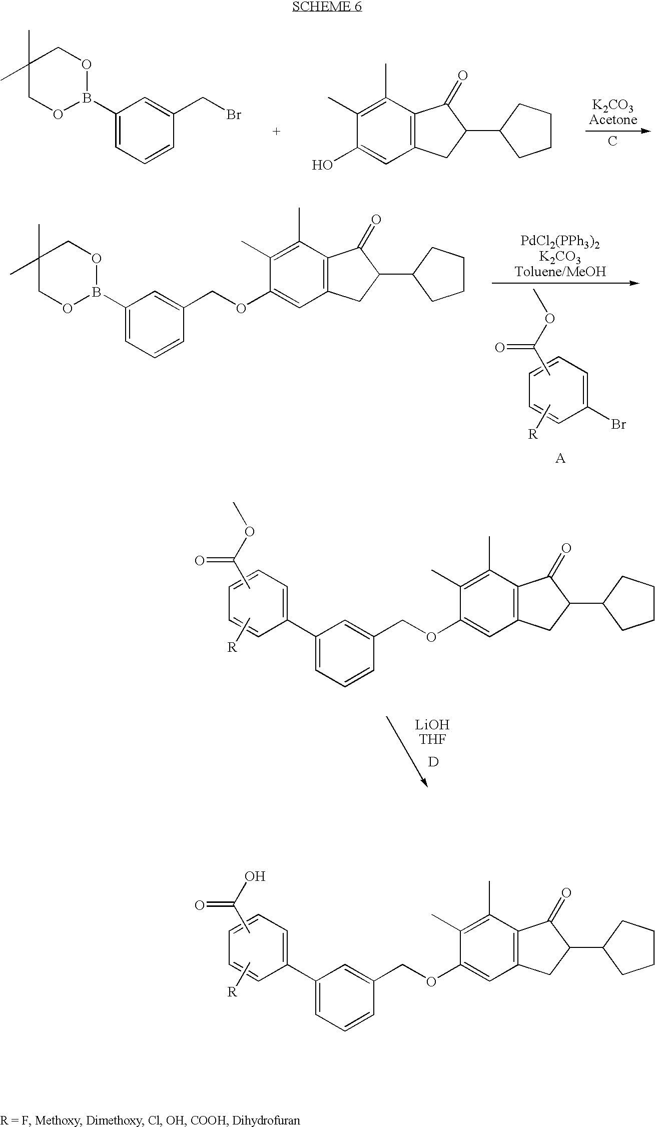

A variety of indanones containing biphenyl linkers can be prepared as outlined in scheme 6. A precursor for transition metal catalyzed cross coupling can be made by an alkylation as shown in step C and as described above using techniques well known in the art. This is then subjected to the cross coupling as illustrated in step A and as described above using techniques well known in the art, either aromatic substitutions or transition metal catalyzed cross couplings following techniques well known in the art. Final ester hydrolysis (step D) gives the desired products as described above using techniques well known in the art. In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLE 1

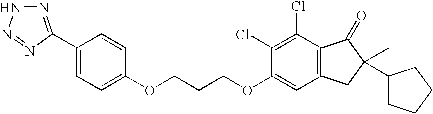

### 6,7-Dichloro-2-cyclopentyl-2-methyl-5-{3-[4-(2H-tetrazol-5-yl)-phenoxy]-propoxy}-indan-1-one

Potassium carbonate (0.91 g, 6.6 mmol) was added to a stirred solution of 6,7-Dichloro-2-cyclopentyl-5-hydroxy-2-methyl-indan-1-one (1.00 g, 3.3 mmol) and 4-(3-Bromo-propoxy)-benzonitrile (1.20 g, 5.0 mmol) in acetone (50 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 4-[3-(6,7-Dichloro-2-cyclopentyl-2-methyl-1-oxo-indan-5-yloxy)-propoxy]-benzonitrile as a white solid. 4-[3-(6,7-Dichloro-2-cyclopentyl-2-methyl-1-oxo-indan-5-yloxy)-propoxy]-benzonitrile (0.250 g, 0.55 mmol), trimethylsilylazide (0.130 g, 0.15 mL, 1.1 mmol) and dibutyltin oxide (20 mg, 0.083 mmol) were dissolved in toluene (15 mL) and heated to reflux for 16 hr. The reaction mixture was then cooled to rt and applied directly to a silica gel column (eluting first with 20% ethyl acetate/hexanes followed by 10% MeOH/dichloromethane) to give of 6,7-Dichloro-2-cyclopentyl-2-methyl-5-{3-[4-(2H-tetrazol-5-yl)-phenoxy]-propoxy}-indan-1-one as a white solid. ¹H NMR (DMSO-d₆, 500 MHz), δ 7.98 (d, 2H), 7.34 (s, 1H), 7.18 (d, 2H), 4.42 (t, 2H), 4.27 (t, 2H), 2.96 (d, 1H), 2.72 (d, 1H), 2.32-2.39 (m, 2H), 2.05 (quint, 1H), 1.74-1.72 (m, 1H), 1.52-1.15 (m, 6H), 1.12 (s, 3H), 0.86-0.81 (m, 1H). MS (ESI): 502 (M+H)⁺.

### EXAMPLE 2

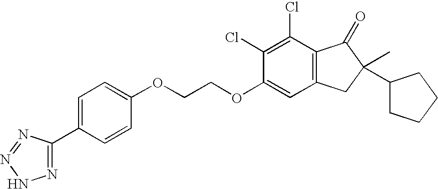

### 6,7-Dichloro-2-cyclopentyl-2-methyl-5-{2-[4-(2H-tetrazol-5-yl)-phenoxy]-ethoxy}-indan-1-one

Potassium carbonate (0.69 g, 5.0 mmol) was added to a stirred solution of 6,7-Dichloro-2-cyclopentyl-5-hydroxy-2-methyl-indan-1-one (0.75 g, 2.5 mmol) and 4-(2-Bromo-ethoxy)-benzonitrile (0.68 g, 3.0 mmol) in acetone (40 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 4-[2-(6,7-Dichloro-2-cyclopentyl-2-methyl-1-oxo-indan-5-yloxy)-ethoxy]-benzonitrile as a white solid. 4-[2-(6,7-Dichloro-2-cyclopentyl-2-methyl-1-oxo-indan-5-yloxy)-ethoxy]-benzonitrile (0.150 g, 0.34 mmol), trimethylsilylazide (0.078 g, 0.09 mL, 0.68 mmol) and dibutyltin oxide (13 mg, 0.051 mmol) were dissolved in toluene (10 mL) and heated to reflux for 16 hr. The reaction mixture was then cooled to rt and applied directly to a silica gel column (eluting first with 20% ethyl acetate/hexanes followed by 10% MeOH/dichloromethane) to give 34 mg (21%) of 6,7-Dichloro-2-cyclopentyl-2-methyl-5-{2-[4-(2H-tetrazol-5-yl)-phenoxy]-ethoxy}-indan-1-one as a white solid. ¹H NMR (DMSO-d₆, 500 MHz), δ 7.98 (d, 2H), 7.42 (s, 1H), 7.21 (d, 2H), 4.62-4.61 (m, 2H), 4.51-4.51 (m, 2H), 3.01 (d, 1H), 2.77 (d, 1H), 2.07 (quint, 1H), 1.74-1.72 (m, 1H), 1.52-1.15 (m, 6H), 1.12 (s, 3H), 0.86-0.81 (m, 1H). MS (ESI): 487 (M+H)⁺.

### EXAMPLE 3

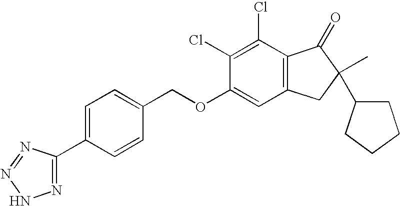

### 6,7-Dichloro-2-cyclopentyl-2-methyl-5-[4-(2H-tetrazol-5-yl)-benzyloxy]-indan-1-one

Potassium carbonate (0.91 g, 6.6 mmol) was added to a stirred solution of 6,7-Dichloro-2-cyclopentyl-5-hydroxy-2-methyl-indan-1-one (1.00 g, 3.3 mmol) and 4-Bromomethyl-benzonitrile (0.98 g, 5.0 mmol) in acetone (50 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 4-(6,7-Dichloro-2-cyclopentyl-2-methyl-1-oxo-indan-5-yloxymethyl)-benzonitrile as a white solid. 4-(6,7-Dichloro-2-cyclopentyl-2-methyl-1-oxo-indan-5-yloxymethyl)-benzonitrile (0.60 g, 1.45 mmol), trimethylsilylazide (0.33 g, 0.39 mL, 2.9 mmol) and dibutyltin oxide (34 mg, 0.22 mmol) were dissolved in toluene (20 mL) and heated to reflux for 16 hr. The reaction mixture was then cooled to rt and applied directly to a silica gel column (eluting first with 20% ethyl acetate/hexanes followed by 10% MeOH/dichloromethane) to give 6,7-Dichloro-2-cyclopentyl-2-methyl-5-[4-(2H-tetrazol-5-yl)-benzyloxy]-indan-1-one as a white solid. ¹H NMR (DMSO-d₆, 500 MHz), δ 8.10 (d, 2H), 7.70 (d, 2H), 7.44 (s, 1H), 5.42 (s, 2H), 3.08 (d, 1H), 2.75 (d, 1H), 2.07 (quint, 1H), 1.74-1.72 (m, 1H), 1.52-1.15 (m, 6H), 1.12 (s, 3H), 0.86-0.81 (m, 1H). MS (ESI): 457 (M+H)⁺.

### EXAMPLE 4

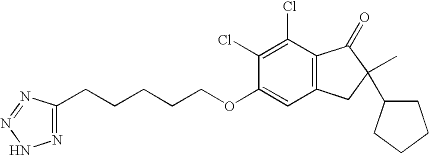

### 6,7-Dichloro-2-cyclopentyl-2-methyl-5-[5-(2H-tetrazol-5-yl)-pentyloxy]-indan-1-one

Potassium carbonate (0.46 g, 3.3 mmol) was added to a stirred solution of 6,7-Dichloro-2-cyclopentyl-5-hydroxy-2-methyl-indan-1-one (0.50 g, 1.65 mmol) and 6-Bromo-hexanenitrile (0.58 g, 0.44 mL, 3.3 mmol) in acetone (50 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 6-(6,7-Dichloro-2-cyclopentyl-2-methyl-1-oxo-indan-5-yloxy)-hexanenitrile as a white solid. 6-(6,7-Dichloro-2-cyclopentyl-2-methyl-1-oxo-indan-5-yloxy)-hexanenitrile (0.60 g, 1.52 mmol), trimethylsilylazide (0.35 g, 0.40 mL, 3.04 mmol) and dibutyltin oxide (57 mg, 0.23 mmol) were dissolved in toluene (20 mL) and heated to reflux for 16 hr. The reaction mixture was then cooled to rt and applied directly to a silica gel column (eluting first with 20% ethyl acetate/hexanes followed by 10% MeOH/dichloromethane) to give 6,7-Dichloro-2-cyclopentyl-2-methyl-5-[5-(2H-tetrazol-5-yl)-pentyloxy]-indan-1-one as a white solid. ¹H NMR (DMSO-d₆, 500 MHz), δ 7.29 (s, 1H), 4.20 (t, 2H), 3.01 (d, 1H), 2.92 (t, 2H), 2.75 (d, 1H), 2.07 (quint, 1H), 1.84-1.76 (m, 5H), 1.52-1.15 (m, 8H), 1.12 (s, 3H), 0.86-0.81 (m, 1H). MS (ESI): 437 (M+H)⁺.

### EXAMPLE 5

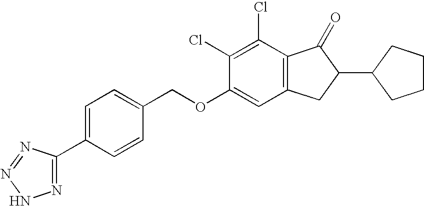

### 6,7-Dichloro-2-cyclopentyl-5-[4-(2H-tetrazol-5-yl)-benzyloxy]-indan-1-one

Potassium carbonate (0.55 g, 4.0 mmol) was added to a stirred solution of 6,7-Dichloro-2-cyclopentyl-5-hydroxy-indan-1-one (0.57 g, 2.0 mmol) and 4-Bromomethyl-benzonitrile (0.47 g, 2.4 mmol) in acetone (25 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 4-(6,7-Dichloro-2-cyclopentyl-1-oxo-indan-5-yloxymethyl)-benzonitrile as a white solid. 4-(6,7-Dichloro-2-cyclopentyl-1-oxo-indan-5-yloxymethyl)-benzonitrile (0.250 g, 0.62 mmol), trimethylsilylazide (0.14 g, 0.17 mL, 1.25 mmol) and dibutyltin oxide (23 mg, 0.093 mmol) were dissolved in toluene (15 mL) and heated to reflux for 16 hr. The reaction mixture was then cooled to rt and applied directly to a silica gel column (eluting first with 20% ethyl acetate/hexanes followed by 10% MeOH/dichloromethane) to give 6,7-Dichloro-2-cyclopentyl-5-[4-(2H-tetrazol-5-yl)-benzyloxy]-indan-1-one as a white solid. ¹H NMR (DMSO-d₆, 500 MHz), δ 8.10 (d, 2H), 7.70 (d, 2H), 7.45 (s, 1H), 5.44 (s, 2H), 3.21-3.17 (m, 1H), 2.82-2.76 (m, 2H), 2.20-2.18 (m, 1H), 1.88-1.86 (m, 1H), 1.52-1.15 (m, 6H), 1.07-1.05 (m, 1H). MS (ESI): 443 (M+H)⁺.

### EXAMPLE 6

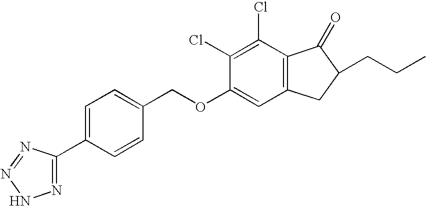

### 6,7-Dichloro-2-propyl-5-[4-(2H-tetrazol-5-yl)-benzyloxy]-indan-1-one

Potassium carbonate (0.55 g, 4.0 mmol) was added to a stirred solution of 6,7-Dichloro-5-hydroxy-2-propyl-indan-1-one (0.52 g, 2.0 mmol) and 4-Bromomethyl-benzonitrile (0.47 g, 2.4 mmol) in acetone (25 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 4-(6,7-Dichloro-1-oxo-2-propyl-indan-5-yloxymethyl)-benzonitrile as a white solid. 4-(6,7-Dichloro-1-oxo-2-propyl-indan-5-yloxymethyl)-benzonitrile (0.150 g, 0.4 mmol), trimethylsilylazide (0.09 g, 0.11 mL, 0.8 mmol) and dibutyltin oxide (15 mg, 0.06 mmol) were dissolved in toluene (8 mL) and heated to reflux for 16 hr. The reaction mixture was then cooled to rt and applied directly to a silica gel column (eluting first with 20% ethyl acetate/hexanes followed by 10% MeOH/dichloromethane) to give 6,7-Dichloro-2-propyl-5-[4-(2H-tetrazol-5-yl)-benzyloxy]-indan-1-one as a white solid. ¹H NMR (DMSO-d₆, 500 MHz), δ 8.04 (d, 2H), 7.69 (d, 2H), 7.43 (s, 1H), 5.40 (s, 2H), 3.35-3.25 (m, 1H), 2.77-2.70 (m, 2H), 1.79-1.73 (m, 1H), 1.41-1.35 (m, 3H), 0.91 (t, 3H). MS (ESI): 417 (M+H)⁺.

### EXAMPLE 7

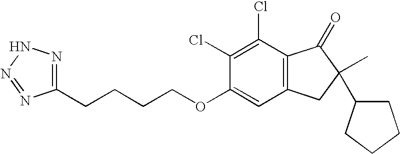

### 6,7-Dichloro-2-cyclopentyl-2-methyl-5-[4-(2H-tetrazol-5-yl)-butoxy]-indan-1-one

Potassium carbonate (0.185 g, 1.34 mmol) was added to a stirred solution of 6,7-Dichloro-2-cyclopentyl-5-hydroxy-2-methyl-indan-1-one (0.20 g, 0.67 mmol) and 5-Bromo-pentanenitrile (0.217 g, 0.16 mL, 1.34 mmol) in acetone (10 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 5-(6,7-Dichloro-2-cyclopentyl-2-methyl-1-oxo-indan-5-yloxy)-pentanenitrile as a white solid. 5-(6,7-Dichloro-2-cyclopentyl-2-methyl-1-oxo-indan-5-yloxy)-pentanenitrile (0.043 g, 0.11 mmol), trimethylsilylazide (0.026 g, 0.03 mL, 0.22 mmol) and dibutyltin oxide (4 mg, 0.017 mmol) were dissolved in toluene (5 mL) and heated to reflux for 16 hr. The reaction mixture was then cooled to rt and applied directly to a silica gel column (eluting first with 20% ethyl acetate/hexanes followed by 10% MeOH/dichloromethane) to give 6,7-Dichloro-2-cyclopentyl-2-methyl-5-[4-(2H-tetrazol-5-yl)-butoxy]-indan-1-one as a white solid. ¹H NMR (DMSO-d₆, 500 MHz), δ 7.30 (s, 1H), 4.36 (t, 2H), 3.05 (d, 1H), 2.95 (t, 2H), 2.76 (d, 1H), 2.05 (quint, 1H), 1.84-1.76 (m, 5H), 1.52-1.15 (m, 6H), 1.12 (s, 3H), 0.86-0.81 (m, 1H). MS (ESI): 423 (M+H)⁺.

### EXAMPLE 8

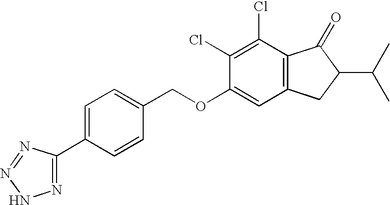

### 6,7-Dichloro-2-isopropyl-5-[4-(2H-tetrazol-5-yl)-benzyloxy]-indan-1-one

Potassium carbonate (0.276 g, 2.0 mmol) was added to a stirred solution of 6,7-Dichloro-5-hydroxy-2-isopropyl-indan-1-one (0.259 g, 1.0 mmol) and 4-Bromomethyl-benzonitrile (0.294 g, 1.5 mmol) in acetone (10 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 310 mg (83%) of 4-(6,7-Dichloro-2-isopropyl-1-oxo-indan-5-yloxymethyl)-benzonitrile as a white solid. 4-(6,7-Dichloro-2-isopropyl-1-oxo-indan-5-yloxymethyl)-benzonitrile (0.300 g, 0.8 mmol), trimethylsilylazide (0.19 g, 0.21 mL, 1.6 mmol) and dibutyltin oxide (30 mg, 0.12 mmol) were dissolved in toluene (15 mL) and heated to reflux for 16 hr. The reaction mixture was then cooled to rt and applied directly to a silica gel column (eluting first with 20% ethyl acetate/hexanes followed by 10% MeOH/dichloromethane) to give 6,7-Dichloro-2-isopropyl-5-[4-(2H-tetrazol-5-yl)-benzyloxy]-indan-1-one as a white solid. ¹H NMR (DMSO-d₆, 500 MHz), δ 8.07 (d, 2H), 7.60 (d, 2H), 7.47 (s, 1H), 5.41 (s, 2H), 3.10 (dd, 1H), 2.78 (dd, 1H), 2.75-2.72 (m, 1H), 2.23-2.21 (r, 1H), 0.98 (d, 3H), 0.73 (d, 3H). MS (ESI): 417 (M+H)⁺.

### EXAMPLE 9

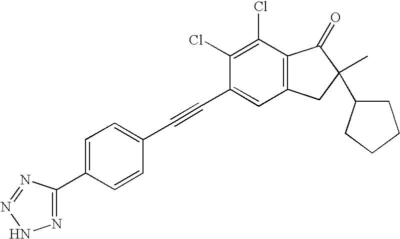

### 6,7-Dichloro-2-cyclopentyl-2-methyl-5-[4-(2H-tetrazol-5-yl)-phenylethynyl]-indan-1-one

Cesium carbonate (0.40 g, 1.5 mmol) was added to a stirred solution of 6,7-Dichloro-2-cyclopentyl-5-hydroxy-2-methyl-indan-1-one (0.30 g, 1 mmol) and N-phenyltriflamide (0.429 g, 1.2 mmol) in methylene chloride/DMF 9/1 (5 mL) at 0° C. The reaction mixture was stirred for 2 hr, then the solvent was removed in vacuo. The residue was then mixed with dichloromethane (200 mL) and water (200 mL). The organic layer was separated, rinsed with aqueous sodium bicarbonate, dried over MgSO₄ and then concentrated in vacuo to give 429 mg (quant) of Trifluoro-methanesulfonic acid 6,7-dichloro-2-cyclopentyl-2-methyl-1-oxo-indan-5-yl ester as a yellow oil. Trifluoro-methanesulfonic acid 6,7-dichloro-2-cyclopentyl-2-methyl-1-oxo-indan-5-yl ester (430 mg, 1 mmol), 4-ethynyl-benzonitrile (178 mg, 1.4 mmol), bis-triphenylphosphino palladium dichloride (28 mg, 0.04 mmol) and copper (I) iodide (4 mg, 0.02 mmol) were mixed in triethylamine (10 mL) and heated to 65° C. for 4 hr. The reaction was cooled to rt and applied directly to a silica gel column (eluting 5-50% ethyl acetate/hexanes) to give 4-(6,7-Dichloro-2-cyclopentyl-2-methyl-1-oxo-indan-5-ylethynyl)-benzonitrile as a yellow oil. 4-(6,7-Dichloro-2-cyclopentyl-2-methyl-1-oxo-indan-5-ylethynyl)-benzonitrile (60 mg, 0.15 mmol), trimethylsilylazide (0.034 g, 0.04 mL, 0.3 mmol) and dibutyltin oxide (6 mg, 0.023 mmol) were dissolved in toluene (5 mL) and heated to reflux for 16 hr. The reaction mixture was then cooled to rt and applied directly to a silica gel column (eluting first with 20% ethyl acetate/hexanes followed by 10% MeOH/dichloromethane) to give 6,7-Dichloro-2-cyclopentyl-2-methyl-5-[4-(2H-tetrazol-5-yl)-phenylethynyl]-indan-1-one as a light yellow solid. ¹H NMR (DMSO-d₆, 500 MHz), δ 8.13 (d, 2H), 7.88 (s, 1H), 7.74 (d, 2H), 3.08 (d, 1H), 2.80 (d, 1H), 2.08 (quint, 1H), 1.84-1.76 (m, 1H), 1.52-1.15 (m, 6H), 1.12 (s, 3H), 0.86-0.81 (m, 1H). MS (ESI): 451 (M+H)⁺.

### EXAMPLE 10

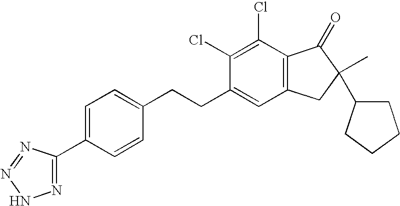

### 6,7-Dichloro-2-cyclopentyl-2-methyl-5-{2-[4-(2H-tetrazol-5-yl)-phenyl]-ethyl}-indan-1-one

6,7-Dichloro-2-cyclopentyl-2-methyl-5-[4-(2H-tetrazol-5-yl)-phenylethynyl]-indan-1-one (43 mg, 0.095 mmol) and 10% palladium on carbon (4 mg) were mixed together in methanol (10 mL) at rt under an atmosphere of hydrogen for 10 hr. The palladium was then filtered off and the solvent removed in vacuo to give 43 mg (quant) of 6,7-Dichloro-2-cyclopentyl-2-methyl-5-{2-[4-(2H-tetrazol-5-yl)-phenyl]-ethyl}-indan-1-one as a white solid. ¹H NMR (DMSO-d₆, 500 MHz), δ 7.91 (d, 2H), 7.56 (s, 1H), 7.72 (d, 2H), 3.17-3.12 (m, 2H), 3.01 (d, 1H), 2.96-2.90 (m, 2H), 2.75 (d, 1H), 2.07 (quint, 1H), 1.84-1.76 (m, 1H), 1.52-1.15 (m, 6H), 1.12 (s, 3H), 0.86-0.81 (m, 1H). MS (ESI): 455 (M+H)⁺.

### EXAMPLE 11

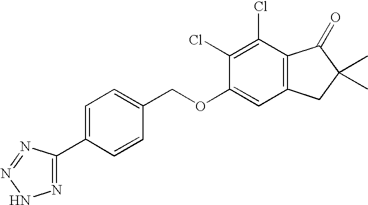

### 6,7-Dichloro-2,2-dimethyl-5-[4-(2H-tetrazol-5-yl)-benzyloxy]-indan-1-one

Potassium carbonate (0.57 g, 4.1 mmol) was added to a stirred solution of 6,7-Dichloro-5-hydroxy-2,2-dimethyl-indan-1-one (0.50 g, 2.05 mmol) and 4-Bromomethyl-benzonitrile (0.60 g, 3.1 mmol) in acetone (25 mL) at 45° C. The reaction mixture was stirred for 16 hr, then the acetone was removed in vacuo. The residue was then mixed with dichloromethane (200 mL) and water (200 mL). The organic layer was separated, dried over MgSO₄ and then concentrated in vacuo to give a residue that was purified via column chromatography on silica gel (eluting 1-30% ethyl acetate/hexanes) to give 4-(6,7-Dichloro-2,2-dimethyl-1-oxo-indan-5-yloxymethyl)-benzonitrile as a white solid. 4-(6,7-Dichloro-2,2-dimethyl-1-oxo-indan-5-yloxymethyl)-benzonitrile (0.300 g, 0.83 mmol), trimethylsilylazide (0.19 g, 0.21 mL, 1.6 mmol) and dibutyltin oxide (30 mg, 0.12 mmol) were dissolved in toluene (10 mL) and heated to reflux for 16 hr. The reaction mixture was then cooled to rt and applied directly to a silica gel column (eluting first with 20% ethyl acetate/hexanes followed by 10% MeOH/dichloromethane) to give 6,7-Dichloro-2,2-dimethyl-5-[4-(2H-tetrazol-5-yl)-benzyloxy]-indan-1-one as a white solid. ¹H NMR (DMSO-d₆, 500 MHz), δ 8.10 (d, 2H), 7.72 (d, 2H), 7.46 (s, 1H), 5.46 (s, 2H), 2.96 (s, 1H), 1.14 (s, 6H). MS (ESI): 403 (M+H)⁺.

### EXAMPLE 12

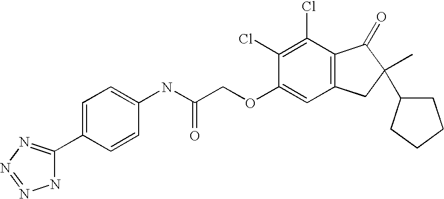

### 2-(6,7-Dichloro-2-cyclopentyl-2-methyl-1-oxo-indan-5-yloxy)-N-[4-(1H-tetrazol-5-yl)-phenyl]-acetamide

5-(4-Nitro-phenyl)-1H-tetrazole (5.3 g, 27.7 mmol), absolute ethanol (50 ml), ethyl acetate (50 ml) and 10% palladium on carbon (500 mg) was placed under hydrogen atmosphere at 30 psi in a Parr for 16 hours. The reaction mixture was filtered through a celite pad washing with ethyl acetate. The filtrate was concentrated in vacuo to give 4-(1H-tetrazol-5-yl)-phenylamine as a light orange solid (4.5 g, 100%). To a stirred mixture of (6,7-dichloro-2-cyclopentyl-2-methyl-1-oxo-indan-5-yloxy)-acetic acid (308 mg, 0.86 mmol) and dichloromethane was added oxalyl chloride (0.4 ml, 4.6 mmol) at room temperature. Dimethylformamide (0.05 ml) was added dropwise to catalyze reaction. Reaction was allowed to stir until no starting material was observed by tlc and then concentrated under reduced pressure to give the acid chloride as a yellow oil. The acid chloride was stirred in dichloromethane (8.0 ml) and 4-(1H-tetrazol-5-yl)-phenylamine (138 mg, 0.86 mmol) at 0° C. under nitrogen. Pyridine (0.15 ml, 1.86 mmol) was added to the mixture and then allowed to warm to room temperature overnight. Reaction was concentrated in vacuo and the resulting oil was purified by flash chromatography on silica gel (0-20% methanol/chloroform, followed by 1:3:96 acetic acid:methanol:chloroform) to give 2-(6,7-dichloro-2-cyclopentyl-2-methyl-1-oxo-indan-5-yloxy)-N-[4-(1H-tetrazol-5-yl)-phenyl]-acetamide as a orange solid. ¹H NMR (DMSO-d₆, 500 MHz), δ 10.56 (s, 1H), 8.01-7.99 (d, 2H), 7.82-7.80 (d, 2H), 7.28 (s, 1H), 5.09 (s, 2H), 3.00-2.97 (d, 1H), 2.77-2.73 (d, 1H), 2.07-2.06 (m, 1H), 1.50-1.51 (m, 1H), 1.46-1.44 (m, 4H), 1.33-1.32 (m, 1H), 1.21-1.20 (m, 1H), 1.13 (s, 3H), 0.86 (m, 1H). (ESI): 500 (M+H)⁺

### EXAMPLE 13

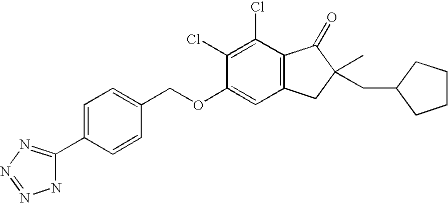

### 6,7-Dichloro-2-cyclopentylmethyl-2-methyl-5-[4-(1H-tetrazol-5-yl)-benzyloxy]-indan-1-one

6,7-Dichloro-2-cyclopentylmethyl-5-hydroxy-2-methyl-indan-1-one (250 mg, 0.80 mmol), α-bromo-p-tolunitrile (235 mg, 1.2 mmol), acetone (11.0 ml) and potassium carbonate (230 mg, 1.7 mmol) was stirred at 40-45° C. under nitrogen overnight. The reaction mixture was washed with water (2×30 ml), extracted with dichloromethane (2×25 ml). The combined organic extracts was dried (Na₂SO₄), filtered and the filtrate was concentrated in vacuo to give a crude solid. Flash chromatography of the solid (10-60% ethyl acetate/hexanes) gave 4-(6,7-dichloro-2-cyclopentylmethyl-2-methyl-1-oxo-indan-5-yloxymethyl)-benzonitrile as a white solid. A mixture of 4-(6,7-dichloro-2-cyclopentylmethyl-2-methyl-1-oxo-indan-5-yloxymethyl)-benzonitrile (250 mg, 0.58 mmol), toluene (8.3 ml), azidotrimethylsilane (0.15 ml, 1.16 mmol) and dibutyl tin oxide (21 mg, 0.09 mmol) was heated to 110° C. overnight under nitrogen atmosphere. The reaction mixture was cooled and purified directly via flash chromatography on silica gel (30-100 ethyl acetate/hexanes, followed by 5-20 methanol/ethyl acetate) to give the target compound as a yellow solid. ¹H NMR (DMSO-d₆, 500 MHz), δ 8.11-8.09 (d, 2H), 7.71-7.68 (d, 2H), 7.47 (s, 2H), 5.44 (s, 2H), 3.15 (d, 1H), 2.89 (d, 1H), 1.69-1.36 (m, 9H), 1.12 (s, 3H), 1.10 (m, 1H), 0.93 (m, 2H). (ESI): 471 M⁺.

### EXAMPLE 14

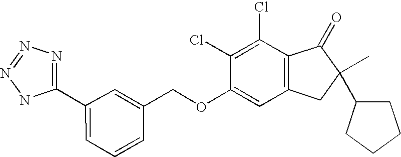

### 6,7-Dichloro-2-cyclopentyl-2-methyl-5-[3-(1H-tetrazol-5-yl)-benzyloxy]-indan-1-one

A mixture of 6,7-dichloro-2-cyclopentylmethyl-5-hydroxy-2-methyl-indan-1-one (250 mg, 0.83 mmol), acetone (12 ml), potassium carbonate (229 mg, 1.66 mol) and α-bromo-m-tolunitrile (245 mg, 1.25 mmol) was heated to 40-45° C. under nitrogen atmosphere overnight. The reaction was washed with water (60 ml) and extracted with dichloromethane (60 ml). The combined organic extracts were dried (Na₂SO₄), filtered and the filtrate concentrated in vacuo to afford 3-(6,7-dichloro-2-cyclopentyl-2-methyl-1-oxo-indan-5-yloxymethyl)-benzonitrile as a white foam. A mixture of 3-(6,7-dichloro-2-cyclopentyl-2-methyl-1-oxo-indan-5-yloxymethyl)-benzonitrile (253 mg, 0.62 mmol), anhydrous toluene (8.7 ml), azidotrimethylsilane (0.16 ml, 1.24 mmol) and dibutyl tin oxide (23 mg, 0.09 mmol) was heated to 110° C. overnight under nitrogen atmosphere. Flash chromatography of cooled reaction mixture (30-100 ethyl acetate/hexanes, then 5-20 methanol/ethyl acetate) afforded 6,7-dichloro-2-cyclopentyl-2-methyl-5-[3-(1H-tetrazol-5-yl)-benzyloxy]-indan-1-one as a yellow solid. ¹H NMR (DMSO-d₆, 500 MHz), δ 8.33 (s, 1H), 8.17 (s, 1H), 8.03-8.01 (m, 1H), 7.69 (m, 2H), 7.46 (s, 1H), 5.45 (d, 2H), 3.03-3.00 (d, 1H), 2.80-2.77 (d, 1H), 1.99 (m, 1H), 1.77 (m, 1H), 1.56-1.45 (m, 4H), 1.34 (m, 1H), 1.18 (m, 1H), 1.14 (s, 3H), 0.86 (m, 1H). (ESI): 457 M⁺.

### EXAMPLE 15

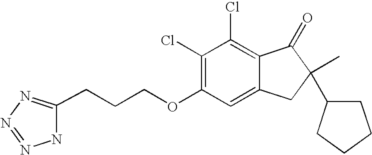

### 6,7-Dichloro-2-cyclopentyl-2-methyl-5-[3-(1H-tetrazol-5-yl)-propoxy]-indan-1-one

A mixture of 6,7-dichloro-2-cyclopentyl-5-hydroxy-2-methyl-indan-1-one (200 mg, 0.67 mmol), 4-bromo-butyronitrile (0.1 ml, 1.0 mmol), acetone (9.6 ml) and potassium carbonate (190 mg, 1.37 mmol) was stirred at 40-45° C. overnight. The reaction mixture was cooled and concentrated in vacuo. The resulting residue was washed with water (2×15 ml) and extracted with dichloromethane (2×20 ml). The organic extracts were dried (Na₂SO₄), filtered and the filtrate concentrated. Flash chromatography of resulting oil (5-50% ethyl acetate/hexanes) afforded 4-(6,7-dichloro-2-cyclopentyl-2-methyl-1-oxo-indan-5-yloxy)-butyronitrile as a white foam. A mixture of 4-(6,7-dichloro-2-cyclopentyl-2-methyl-1-oxo-indan-5-yloxy)-butyronitrile (168 mg, 0.46 mmol), toluene (6.5 ml), azidotrimethylsilane (0.12 ml, 0.92 mmol) and dibutyl tin oxide (17 mg, 0.07 mmol) was heated to 110° C. under nitrogen atmosphere overnight. The cooled reaction mixture was purified directly via flash chromatography on silica gel (20-100 ethyl acetate/hexanes) to give 6,7-dichloro-2-cyclopentyl-2-methyl-5-[3-(1H-tetrazol-5-yl)-propoxy]-indan-1-one as a white solid. ¹H NMR (DMSO-d₆, 500 MHz), δ 4.30 (m, 2H), 3.09 (m, 2H), 3.01 (d, 1H), 2.77 (d, 1H), 2.26 (m, 2H), 2.10 (m, 1H), 1.90 (m, 1H), 1.45-1.40 (m, 4H), 1.35 (m, 1H), 1.25 (m, 1H), 1.13 (s, 3H), 0.90 (m, 1H). (ESI) 409 M⁺

### EXAMPLE 16

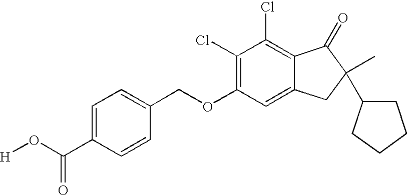

### 4-(6,7-Dichloro-2-cyclopentyl-2-methyl-1-oxo-indan-5-yloxymethyl)-benzoic acid

A mixture of 6,7-dichloro-2-cyclopentyl-5-hydroxy-2-methyl-indan-1-one (150 mg, 0.5 mmol), methyl-4-(bromomethyl)benzoate (230 mg, 1.0 mmol), acetone (7.2 ml) and potassium carbonate (100 mg, 0.72 mmol) was heated overnight at 40-45° C. under nitrogen atmosphere. The reaction mixture was concentrated in vacuo, washed with water (20 ml) and extracted with dichloromethane (40 ml). The organic extracts were combined, dried (Na₂SO₄), filtered and the filtrate was concentrated. Flash chromatography of the resulting residue (10-60% ethyl acetate/hexanes) afforded 4-(6,7-dichloro-2-cyclopentyl-2-methyl-1-oxo-indan-5-yloxymethyl)-benzoic acid methyl ester as a white foam. A mixture of 4-(6,7-dichloro-2-cyclopentyl-2-methyl-1-oxo-indan-5-yloxymethyl)-benzoic acid methyl ester (147 mg, 0.32 mmol), tetrahydrofuran (1.6 ml) and 1.0 N aqueous lithium hydroxide (1.0 ml) was vigorously stirred at 45° C. until no starting material was observed by tlc. The reaction mixture was cooled and taken to pH 6 by addition of 1.0 M aqueous HCl. The mixture was washed with water and extracted with ethyl acetate. The combined organic extracts were dried (Na₂SO₄), filtered and the filtrate was concentrated to give 4-(6,7-dichloro-2-cyclopentyl-2-methyl-1-oxo-indan-5-yloxymethyl)-benzoic acid as a fine white powder. ¹H NMR (DMSO-d₆, 500 MHz), δ 8.00 (d, 2H), 7.67 (d, 2H), 7.46 (s, 1H), 5.44 (s, 2H), 3.02 (d, 1H), 2.78 (d, 1H), 2.07 (m, 1H), 1.70 (m, 1H), 1.49-1.42 (m, 4H), 1.33 (m, 1H), 1.21 (m, 1H), 1.14 (s, 3H), 0.84 (m, 1H). (ESI) 433 M⁺.

### EXAMPLE 17

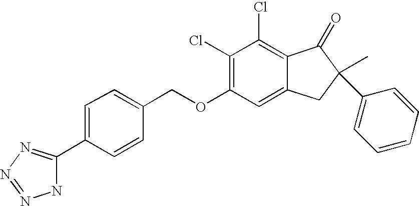

### 6,7-Dichloro-2-methyl-2-phenyl-5-[4-(1H-tetrazol-5-yl)-benzyloxy]-indan-1-one

A mixture of 6,7-dichloro-5-hydroxy-2-methyl-2-phenyl-indan-1-one (500 mg, 1.62 mmol), acetone (26 ml), potassium carbonate (450 mg, 3.25 mmol) and α-bromo-p-tolunitrile (480 mg, 2.45 mmol) was stirred overnight at 40-45° C. The reaction mixture was cooled and concentrated under reduced pressure. The resulting crude solid was purified by flash chromatography (10-90% ethyl acetate/hexanes) to afford 4-(6,7-dichloro-2-methyl-1-oxo-2-phenyl-indan-5-yloxymethyl)-benzonitrile as a white solid. A mixture of -(6,7-dichloro-2-methyl-1-oxo-2-phenyl-indan-5-yloxymethyl)-benzonitrile (291 mg, 0.69 mmol), anhydrous toluene (9.8 ml), azidotrimethyl silane (0.18 ml, 1.38 mmol) and dibutyl tin oxide (26 mg, 0.1 mmol) was stirred at 110° C. overnight under nitrogen atmosphere. The reaction mixture was cooled and purified by flash chromatography on silica gel (2-100% ethyl acetate/hexanes, followed by 5-20 methanol/ethyl acetate) to afford 6,7-dichloro-2-methyl-2-phenyl-5-[4-(1H-tetrazol-5-yl)-benzyloxy]-indan-1-one as a yellow solid. ¹H NMR (DMSO-d₆, 500 MHZ), δ 8.10 (d, 2H), 7.67 (d, 2H), 7.55 (s, 1H), 7.34-7.22 (m, 5H), 5.46 (s, 2H), 3.51 (d, 2H), 3.29 (d, 2H), 1.58 (s, 3H). (ESI) 465 M⁺.

### EXAMPLE 18

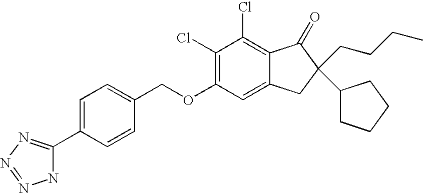

### 2-Butyl-6,7-dichloro-2-cyclopentyl-5-[4-(1H-tetrazol-5-yl)-benzyloxy]-indan-1-one

A mixture of 2-butyl-6,7-dichloro-2-cyclopentyl-5-hydroxy-indan-1-one (500 mg, 1.46 mmol), acetone (20 ml), potassium carbonate (410 mg, 2.9 mmol) and α-bromo-p-tolunitrile (420 mg, 2.14 mmol) was heated at 40-45° C. until no starting material was observed by tlc. The reaction mixture was cooled and concentrated in vacuo. The resulting solid was washed with water (2×20 ml) and extracted with dichloromethane (2×25 ml). The combined organic extracts were dried (Na₂SO₄), filtered and the filtrate concentrated in vacuo. The resulting clear oil was purified by flash chromatography on silica gel (10-60% ethyl acetate/hexanes) to give 4-(2-butyl-6,7-dichloro-2-cyclopentyl-1-oxo-indan-5-yloxymethyl)-benzonitrile as a waxy solid. A mixture of 4-(2-butyl-6,7-dichloro-2-cyclopentyl-1-oxo-indan-5-yloxymethyl)-benzonitrile (562 mg, 1.23 mmol), anhydrous toluene (17.6 ml), azidotrimethyl silane (0.33 ml, 2.46 mmol) and dibutyl tin oxide (46 mg, 0.18 mmol) was heated to 110° C. overnight under nitrogen atmosphere. The reaction mixture was cooled to room temperature and purified by flash chromatography (2-100% ethyl acetate/hexanes, followed by 5-20 methanol/ethyl acetate) to afford 2-butyl-6,7-dichloro-2-cyclopentyl-5-[4-(1H-tetrazol-5-yl)-benzyloxy]-indan-1-one as a yellow solid. ¹H NMR (DMSO-d₆, 500 MHz), δ 8.11 (d, 2H), 7.71 (d, 2H), 7.47 (s, 1H), 5.49 (s, 2H), 2.92 (dd, 2H), 2.15 (m, 1H), 1.70 (m, 1H), 1.65-1.40 (m, 6H), 1.30 (m, 1H), 1.16 (m, 3H), 1.05 (m, 1H), 0.80 (m, 2H), ), 0.79-0.76 (t, 3H). (ESI): 499 M⁺.

### EXAMPLE 19

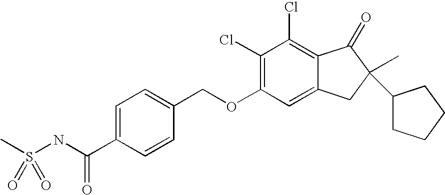

### N-[4-(6,7-Dichloro-2-cyclopentyl-2-methyl-1-oxo-indan-5-yloxymethyl)-benzoyl]-methanesulfonamide

Freshly distilled oxalyl chloride (0.05 ml, 0.57 mmol) was added to a mixture of 4-(6,7-dichloro-2-cyclopentyl-2-methyl-1-oxo-indan-5-yloxymethyl)-benzoic acid (51 mg, 0.12 mmol) and dichloromethane (3.0 ml) at room temperature under nitrogen atmosphere. Dimethylformamide (0.01 ml) was added to catalyze reaction. The mixture was allowed to stir at room temperature until no starting material was detected by tlc. The reaction was then concentrated in vacuo and the resulting acid chloride was dissolved in anhydrous tetrahydrofuran (2.5 ml). In a separate round bottom flask, sodium hydride (8.4 mg, 0.35 mmol) was added to a cooled mixture of methane sulfonamide (30 mg, 0.31 mmol) and anhydrous tetrahydrofuran (1.5 ml) at 0° C. under nitrogen atmosphere. The acid chloride solution was then added to the cooled reaction mixture and allowed to warm to room temperature. Over the next 24 hours, an excess amount of sodium hydride (4-6 eq) was needed to complete reaction. The reaction mixture was washed with brine (25 ml) and extracted with ethyl acetate (60 ml). The combined organic extracts were dried (Na₂SO₄), filtered and concentrated in vacuo. The resulting crude solid was purified by flash chromatography on silica gel (30-100% ethyl acetate/hexanes, followed by 5% methanol/ethyl acetate) to afford N-[4-(6,7-dichloro-2-cyclopentyl-2-methyl-1-oxo-indan-5-yloxymethyl)-benzoyl]-methanesulfonamide as a light yellow solid. ¹H NMR (DMSO-d₆) δ 8.02 (d, 2H), 7.48 (d, 2H), 7.43 (s, 1H), 5.39 (s, 2H), 3.02 (d, 1H), 2.91 (s, 3H), 2.79 (d, 1H), 2.08 (m, 1H), 2.75 (m, 1H), 2.60-1.44 (m, 4H), 1.33 (m, 1H), 1.21 (m, 1H), 1.14 (s, 3H), 0.85 (m, 1H). (ESI): 510M⁺.

### EXAMPLE 20

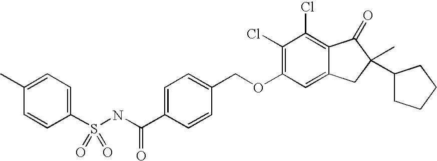

### N-[4-(6,7-Dichloro-2-cyclopentyl-2-methyl-1-oxo-indan-5-yloxymethyl)-benzoyl]-4-methyl-benzenesulfonamide

Freshly distilled oxalyl chloride (0.05 ml, 0.57 mmol) was added to a mixture of 4-(6,7-dichloro-2-cyclopentyl-2-methyl-1-oxo-indan-5-yloxymethyl)-benzoic acid (79 mg, 0.18 mmol) and dichloromethane (4.6 ml) at room temperature under nitrogen atmosphere. Dimethylformamide (0.01 ml) was added to catalyze reaction. Mixture was allowed to stir until no starting material was detected by tlc. The reaction was concentrated in vacuo and the resulting acid chloride was dissolved in anhydrous tetrahydrofuran (2.0 ml). In a separate round bottom flask, sodium hydride (17 mg, 0.54 mmol) was added to a cooled mixture of p-toluene sulfonamide (80 mg, 0.47 mmol) and anhydrous tetrahydrofuran (2.0 ml) at 0° C. under nitrogen atmosphere. The acid chloride solution was then added to the cooled reaction mixture and allowed to warm to room temperature until no acid chloride was observed by tlc. The reaction mixture was washed with brine (25 ml) and extracted with ethyl acetate (60 ml). The combined organic extracts were dried (Na₂SO₄), filtered and concentrated in vacuo. The resulting crude solid was purified by flash chromatography on silica gel (30-100% ethyl acetate/hexanes, followed by 5% methanol/ethyl acetate) to afford N-[4-(6,7-dichloro-2-cyclopentyl-2-methyl-1-oxo-indan-5-yloxymethyl)-benzoyl]-4-methyl-benzenesulfonamide as a white solid. ¹H NMR (DMSO-d₆, 500 MHz) δ 7.93 (d, 2H), 7.72 (d, 2H), 7.43 (d, 2H), 7.39 (s, 1H), 7.19 (d, 2H), 5.41 (s, 2H), 3.01 (d, 1H), 2.77 (d, 1H), 2.32 (s, 3H), 2.09 (m, 1H), 1.75 (m, 1H), 1.54-1.41 (m, 4H), 1.32 (m, 1H), 1.22 (m, 1H), 1.12 (s, 3H), 0.84 (m, 1H). (ESI): 586 M⁺

### EXAMPLE 21

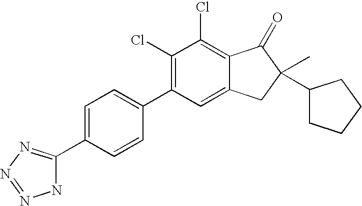

### 6,7-Dichloro-2-cyclopentyl-2-methyl-5-[4-(1H-tetrazol-5-yl)-phenyl]-indan-1-one

A mixture of 6,7-dichloro-2-cyclopentyl-5-hydroxy-2-methyl-indan-1-one (250 mg, 0.83 mmol), anhydrous acetonitrile (7.6 ml) and anhydrous dimethylformamide (0.75 ml) was stirred at 0° C. under nitrogen atmosphere. To the mixture was added N-phenyl-bis(trifluoromethane) sulfonimide (326 mg 0.91 mmol) and then cesium carbonate (296 mg, 0.91 mmol). The reaction was allowed to warm to room temperature and stirred until no starting material was observed by tlc. The reaction was washed with brine (30 ml) and extracted with ethyl acetate (60 ml). The combined organic extracts were dried (Na₂SO₄), filtered and concentrated. The resulting yellow oil was purified by flash chromatography on silica gel (0-50% ethyl acetate/hexanes) to give trifluoro-methanesulfonic acid 6,7-dichloro-2-cyclopentyl-2-methyl-1-oxo-indan-5-yl ester as a clear oil. Sodium carbonate (124 mg, 1.17 mmol) was added to a stirred mixture of trifluoro-methanesulfonic acid 6,7-dichloro-2-cyclopentyl-2-methyl-1-oxo-indan-5-yl ester (317 mg, 0.73 mmol), ethylene glycol dimethyl ether (1.5 ml), palladium tetrakis triphenyl phosphine (25 mg, 0.02 mmol) and water (1.0 ml) at room temperature. 4-Cyanophenyl boronic acid (128 mg, 0.88 mmol) in ethylene glycol dimethyl ether (1.0 ml) was then added to the reaction. Mixture was heated overnight at 85° C. The reaction mixture was cooled, washed with brine (15 ml) and extracted with ethyl acetate (50 ml). The combined organic extracts were dried (Na₂SO₄), filtered and concentrated. The resulting oil was purified by flash chromatography on silica gel (0-20% ethyl acetate/hexanes) to afford 4-(6,7-dichloro-2-cyclopentyl-2-methyl-1-oxo-indan-5-yl)-benzonitrile as a clear oil. A mixture of 4-(6,7-dichloro-2-cyclopentyl-2-methyl-1-oxo-indan-5-yl)-benzonitrile (42 mg, 0.1 μmol), anhydrous toluene (1.6 ml), azidotrimethyl silane (0.03 ml, 0.22 mmol) and dibutyl tin oxide (5.0 mg, 0.02 mmol) was stirred at 110° C. under nitrogen atmosphere. Four equivalents of azidotrimethyl silane over the next two hours was needed to complete reaction. The reaction mixture was cooled to room temperature and purified by flash chromatography on silica gel (30-100% ethyl acetate/hexanes, followed by 5-25% methanol/ethyl acetate) to give 6,7-dichloro-2-cyclopentyl-2-methyl-5-[4-(1H-tetrazol-5-yl)-phenyl]-indan-1-one as a white solid. ¹H NMR (DMSO-d₆), δ 8.17 (d, 2H), 7.71 (d, 2H), 7.66 (s, 1H), 3.12 (d, 1H), 2.87 (d, 1H), 2.13 (m, 1H), 1.79 (m, 1H), 1.57-1.46 (m, 4H), 1.41 (m, 1H), 1.24 (m, 1H), 1.18 (s, 3H), 0.90 (m, 1H).

### EXAMPLE 22

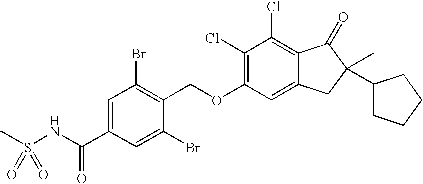

### 3,5-dibromo-4-{[(6,7-dichloro-2-cyclopentyl-2-methyl-1-oxo-2,3-dihydro-1H-inden-5-yl)oxy]methyl}-N-(methylsulfonyl)benzamide

Oxalyl chloride (0.1 ml, 1.2 mmol) was added to a mixture of 3,5-dibromo-4-{[(6,7-dichloro-2-cyclopentyl-2-methyl-1-oxo-2,3-dihydro-1H-inden-5-yl)oxy]methyl}benzoic acid (230 mg, 0.4 mmol) in dichloromethane (9.6 ml) at room temperature under nitrogen atmosphere. Dimethylformamide (0.02 ml) was added and mixture was allowed to stir until no starting material was observed by tlc. The reaction mixture was concentrated in vacuo to give a yellow foam which was dissolved in tetrahydrofuran (4.0 ml). The acid chloride was added to a cooled mixture of methane sulfonamide (91 mg, 0.9 mmol), sodium hydride (60 mg, 1.5 mmol) in tetrahydrofuran (2.0 ml) at 0° C. The reaction mixture was allowed to warn to room temperature and stirred overnight. The mixture was quenched with water, washed with brine and extracted with ethyl acetate. The combined organic extracts were dried over sodium sulfate, filtered and concentrated to give a white solid. Flash chromatography of crude material on silica gel (100% ethyl acetate followed by 0-20% methanol/ethyl acetate) gave the desired product as a white solid. ¹H NMR (DMSO-d₆, 300 MHz) δ 8.17 (s, 2H), 7.61 (s, 1H), 5.48 (s, 2H), 3.12-2.81 (m, 2H), 2.86 (s, 3H), 2.13-2.06 (m, 1H), 1.70-1.77 (m, 1H), 1.56-1.44 (m, 4H), 1.36-1.34 (m, 1H), 1.25-1.19 (m, 1H), 1.16 (s, 3H), 0.95-0.87 (m, 1H).

### EXAMPLE 23

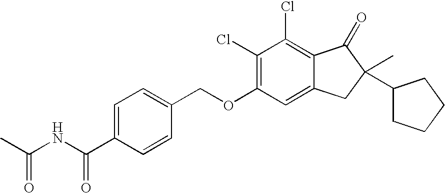

### N-acetyl-4-{[(6,7-dichloro-2-cyclopentyl-2-methyl-1-oxo-2,3-dihydro-1H-inden-5-yl)oxy]methyl}benzamide

p-Toluamide (1.0 g, 7.4 mmol), benzoyl peroxide (110 mg, 0.45 mmol), N-bromosuccinimide (1.7 g, 9.6 mmol) was stirred in carbon tetrachloride (38 ml) at 85° C. overnight under nitrogen atmosphere. The mixture was placed under UV lamp to complete reaction. After cooling mixture to 0° C., the reaction mixture was filtered through celite. The filtrate was washed with water, extracted with dichloromethane and the combined organic extracts were dried over sodium sulfate, filtered and concentrated. The crude material was purified by flash chromatography on silica gel (0-85% ethyl acetate/hexanes) to give 4-(bromomethyl)benzamide (198 mg). A mixture of 6,7-dichloro-2-cyclopentyl-5-hydroxy-2-methylindan-1-one (107 mg, 0.4 mmol), 4-(bromomethyl)benzamide (99 mg, 0.46 mmol), potassium carbonate (72 mg, 0.5 mmol) and acetone (6.0 ml) was stirred at 45° C. overnight. The reaction mixture was concentrated in vacuo to give a crude solid which was purified by flash chromatography on silica gel (30-100% ethyl acetate/hexanes followed by 0-10% methanol/ethyl acetate) to afford 4-{[(6,7-dichloro-2-cyclopentyl-2-methyl-1-oxo-2,3-dihydro-1H-inden-5-yl)oxy]methyl}benzamide (98 mg). Sodium hydride (20 mg, 0.5 mmol) was added to a mixture of 4-{[(6,7-dichloro-2-cyclopentyl-2-methyl-1-oxo-2,3-dihydro-1H-inden-5-yl)oxy]methyl}benzamide (50 mg, 0.11 mmol) and tetrahydrofuran (1.1 ml) at 0° C. Dimethylformamide (1.5 ml) was added to help dissolve any solids. After two hours, no starting material was observed by tlc. Reaction mixture was cooled, quenched with water, washed with brine and extracted with ethyl acetate. The combined organic extracts were dried over sodium sulfate, filtered and concentrated. The crude oil was purified by flash chromatography on silica gel (15-100% ethyl acetate/hexanes followed by 5% methanol/ethyl acetate) to give the desired product as a beige solid. ¹H NMR (DMSO-d₆, 300 MHz) δ 11.05 (s, 1H), 7.97 (d, 2H), 7.63 (d, 2H), 7.43 (s, 1H), 5.44 (s, 2H), 3.02-2.98 (d, 1H), 2.78-2.75 (d, 1H), 2.35 (s, 3H), 2.09-2.06 (m, 1H), 1.76-1.74 (m, 1H), 1.53-1.41 (m, 4H), 1.33-1.89 (m, 3H), 1.14 (s, 3H), 0.87 (m, 1H). MS (ESI) 497, 496 (M⁺+Na), 474 (M⁺).

### EXAMPLE 24

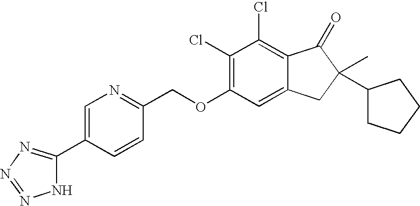

### 6,7-dichloro-2-cyclopentyl-2-methyl-5-{[5-(1H-tetrazol-5-yl)pyridin-2-yl]methoxy}indan-1-one

A mixture of 6,7-dichloro-2-cyclopentyl-5-hydroxy-2-methylindan-1-one (170 mg, 0.6 mmol), 5-bromo-2-(bromomethyl)pyridine (174 mg, 0.7 mmol), potassium carbonate (125 mg, 0.9 mmol) and acetone (9.5 ml) was heated to 40° C. overnight. The reaction mixture was cooled to room temperature and concentrated in vacuo. The resulting solid was washed with brine and extracted with dichloromethane. The combined organic extracts were dried over sodium sulfate, filtered and concentrated to give a crude material which was purified by flash chromatography on silica gel (0-40% ethyl acetate/hexanes). This afforded 5-[(5-bromopyridin-2-yl)methoxy]-6,7-dichloro-2-cyclopentyl-2-methylindan-1-one (260 mg.) A mixture of 5-[(5-bromopyridin-2-yl)methoxy]-6,7-dichloro-2-cyclopentyl-2-methylindan-1-one (101 mg, 0.22 mmol), dimethylformamide (1.2 ml), zinc (II) cyanide (18 mg, 0.15 mmol), tris(dibenzylideneacetone)dipalladium (0) (9.2 mg, 0.01 mmol) and 1,1′-bis (diphenylphosphino)-ferrocene (10.0 mg, 0.02 mmol) was heated to 150° C. for fifteen minutes in a Smith Creator microwave apparatus. Reaction mixture was washed with brine, extracted with ethyl acetate and the combined organic extracts were dried over sodium sulfate and filtered. The collected filtrate was concentrated in vacuo to give 6-{[(6,7-dichloro-2-cyclopentyl-2-methyl-1-oxo-2,3-dihydro-1H-inden-5-yl)oxy]methyl}nicotinonitrile (52 mg). A mixture of 6-{[(6,7-dichloro-2-cyclopentyl-2-methyl-1-oxo-2,3-dihydro-1H-inden-5-yl)oxy]methyl}nicotinonitrile (76 mg, 0.2 mmol), toluene (2.6 ml), azidotrimethylsilane (0.05 ml, 0.36 mmol) and dibutyl tin oxide (8.0 mg, 0.03 mmol) was heated to 110° C. overnight. Flash chromatography on silica gel (50-100% ethyl acetate/hexanes followed by 0-25% methanol/ethyl acetate) afforded the desired product as a tan solid. ¹H NMR (DMSO-d₆, 300 MHz) δ 9.16 (m, 1H), 8.35 (dd, 1H), 7.63 (d, 1H), 7.46 (s, 1H), 5.44 (s, 2H), 2.80 (d, 1H), 2.79 (d, 1H), 2.06-2.05 (m, 1H), 1.76-1.74 (m, 1H), 1.55-1.42 (m, 4H), 1.33-1.32 (m, 1H), 1.21-1.19 (m, 1H), 1.12 (s, 3H), 0.88-0.86 (m, 1H). MS (ESI) 460 (M⁺+2H), 458 (M⁺).

### EXAMPLE 25

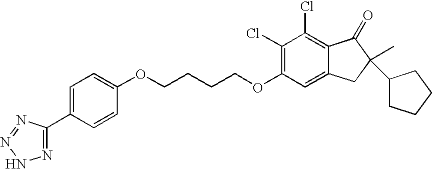

### 6,7-dichloro-2-cyclopentyl-2-methyl-5-{4-[4-(2H-tetrazol-5-yl)phenoxy]butoxy} indan-1-one

A similar procedure as outlined in example 1 was followed using 4-(4-bromobutoxy)benzonitrile. ¹H NMR (DMSO-d₆, 500 MHz) δ 7.95 (d, 2H), 7.31 (s, 1H), 7.12 (d, 2H), 4.31 (t, 2H), 4.16 (t, 2H), 2.75 (d, 1H), 2.51 (d, 1H), 2.08-2.05 (m, 1H), 2.00-1.95 (m, 4H), 1.77-1.44 (m, 1H), 1.54-1.17 (m, 6H), 1.14 (s, 3H), 0.87-0.82 (m, 1H). (ESI): 515 M⁺

### EXAMPLE 26

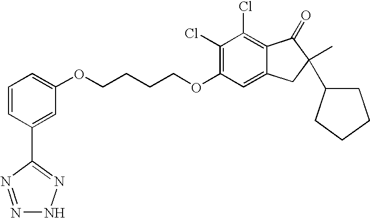

### 6,7-dichloro-2-cyclopentyl-2-methyl-5-{4-[3-(2H-tetrazol-5-yl)phenoxy]butoxy}indan-1-one

A similar procedure as outlined in Example 1 was followed using 3(4-bromobutoxy)benzonitrile. ¹H NMR (DMSO-d₆, 500 MHz) δ 7.63-7.59 (m, 2H), 7.49 (t, 1H), 7.33 (s, 1H), 7.12-7.10 (m, 1H), 4.31 (t, 2H), 4.17 (t, 2H), 2.75 (d, 1H), 2.50 (d, 1H), 2.09-2.05 (m, 1H). 2.00-1.96 (m. 4H). 1.77-1.44 (m, 1H), 1.54-1.17 (m. 6H), 1.14 (s, 3H), 0.87-0.84 (m, 1H). (ESI): 515 M⁺

### EXAMPLE 27

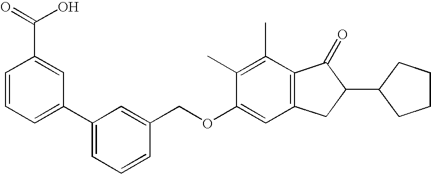

### 3′-{[(2-Cyclopentyl-6,7-dimethyl-1-oxo-2,3-dihydro-1H-inden-5-yl)oxy]methyl}biphenyl-3-carboxylic acid

### General Procedure A: Suzuki Coupling.

A mixture of ethyl-3-bromobenzoate (3 g, 13.1 mmol), [3-(hydroxymethyl)phenyl]-boronic acid (3 g, 19.6 mmol), PdCl₂(PPh₃)₂ (0.46 g, 0.66 mmol), and potassium carbonate (3.6 g, 26.2 mmol) in Toluene/MeOH (10:1, 40 mL) was stirred at 80 C for 18 h. The resulting black mixture was cooled to room temperature, filtered through celite, and poured into a EtOAc/brine mixture. The two layers were separated and the aqueous was extracted with EtOAc (3×). The organics were combined, dried over sodium sulfate, filtered, and evaporated to dryness. The residue was purified by flash chromatography on silica gel eluting with a mixture of EtOAc/Hexane to yield 3 g of ethyl 3′-(hydroxymethyl)biphenyl-3-carboxylate as an orange oil.

### General Procedure B: Benzyl Bromide Formation

A mixture of ethyl 3′-(hydroxymethyl)biphenyl-3-carboxylate (3 g, 11.7 mmol) and triphenyl phosphine (4.6 g, 17.5 mmol) in CH₂Cl₂ (50 mL) was cooled to 4 C. Carbon tetrabromide (5.8 g, 17.5 mmol) dissolved in CH₂Cl₂ (20 mL) was then added dropwise and the resulting orange mixture was stirred at room temperature for 2 h. The solvent was removed and the residue was purified by flash chromatography on silica gel eluting with a mixture of EtOAc/Hexane to yield 3 g of ethyl 3′-(bromomethyl)biphenyl-3-carboxylate as a clear oil.

### General Procedure C: Alkylation

A mixture of ethyl 3′-(bromomethyl)biphenyl-3-carboxylate (300 mg, 0.94 mmol), 2-cyclopentyl-5-hydroxy-6,7-dimethylindan-1-one (183 mg, 0.75 mmol), and potassium carbonate (194 mg, 1.41 mmol) in Acetone (5 mL) was stirred at 50 C for 18 h. The mixture was cooled to room temperature and the Potassium carbonate was removed by filtration. The filtrate was evaporated to dryness and the residue was purified by flash chromatography on silica gel eluting with a mixture of EtOAc/Hexane to yield 300 mg of ethyl 3′-{[(2-cyclopentyl-6,7-dimethyl-1-oxo-2,3-dihydro-1H-inden-5-yl)oxy]methyl}biphenyl-3-carboxylate as a clear oil.

### General Procedure D: Ester Hydrolysis

An aqueous LiOH solution (1M in H₂O, 2 mL) was added dropwise to a solution of Ethyl 3′-{[(2-cyclopentyl-6,7-dimethyl-1-oxo-2,3-dihydro-1H-inden-5-yl)oxy]methyl}biphenyl-3-carboxylate (300 mg, 0.62 mmol) in THF (10 mL). The resulting mixture was heated to 50 C for 48 h. An aqueous HCl (1M, 10 mL) was poured into a cooled reaction mixture and the aqueous phase was extracted with EtOAc (3×). The organics were combined, dried over sodium sulfate, filtered, and evaporated to dryness to afford 245 mg of 3′-{[(2-cyclopentyl-6,7-dimethyl-1-oxo-2,3-dihydro-1H-inden-5-yl)oxy]methyl}biphenyl-3-carboxylic acid as a white solid. ¹H NMR (DMSO-d₆, 500 MHz) δ 12.88 (bs, 1H), 7.99 (s, 1H), 7.72 (m, 2H), 7.59 (s, 1H), 7.45 (d, 1H), 7.39 (t, 1H), 7.30 (m, 2H), 6.84 (s, 1H), 5.07 (s, 2H), 2.83 (m, 1H), 2.40 (m, 2H), 2.29 (s, 3H), 1.95 (m, 1H), 1.90 (s, 3H), 1.59 (m, 1H), 1.36-1.21 (m, 5H), 1.12 (m, 1H), 0.79 (m, 1H). MS (ESI⁺) 455 (M⁺+1).

### EXAMPLE 28

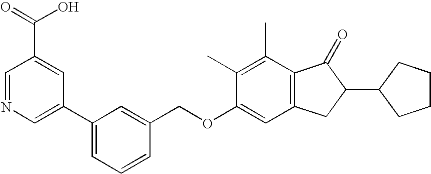

### 5-(3-{[(2-Cyclopentyl-6,7-dimethyl-1-oxo-2,3-dihydro-1H-inden-5-yl)oxy]methyl}phenyl)nicotinic acid

Ethyl 5-[3-(hydroxymethyl)phenyl]nicotinate was synthesized as described in general procedure A.

### General Procedure E: Mitsonobu Coupling

At mixture of Ethyl 5-[3-(hydroxymethyl)phenyl]nicotinate (400 mg, 1.56 mmol), 2-cyclopentyl-5-hydroxy-6,7-dimethylindan-1-one (568 mg, 2.33 mmol), di-tert-butyl (E)-diazene-1,2-dicarboxylate (713 mg, 3.12 mmol), and triphenyl phosphine (812 mg, 3.12 mmol) in THF (10 mL) was stirred at room temperature for 18 h. The solvent was removed and the residue was purified by flash chromatography on silica gel eluting with a mixture of EtOAc/Hexane to yield 535 mg of ethyl 5-(3-{[(2-cyclopentyl-6,7-dimethyl-1-oxo-2,3-dihydro-1H-inden-5-yl)oxy]methyl}phenyl)nicotinate as a yellow oil. 5-(3-{([(2-Cyclopentyl-6,7-dimethyl-1-oxo-2,3-dihydro-1H-inden-5-yl)oxy]methyl}phenyl)nicotinic acid was synthesized as described in general procedure D. ¹H NMR (DMSO-d₆, 500 MHz) δ 9.14 (s, 1H), 9.08 (s, 1H), 8.50 (s, 1H), 7.92 (s, 1H), 7.80 (s, 1H), 7.59 (d, 2H), 7.08 (s, 1H), 5.31 (s, 2H), 3.10 (m, 1H), 2.64 (m, 2H), 2.53 (s, 3H), 2.19 (m, 1H), 2.13 (s, 3H), 1.84 (m, 1H), 1.58-1.38 (m, 5H), 1.36 (m, 1H), 1.02 (m, 1H). MS (ESI⁺) 456 (M⁺+1).

### EXAMPLE 29

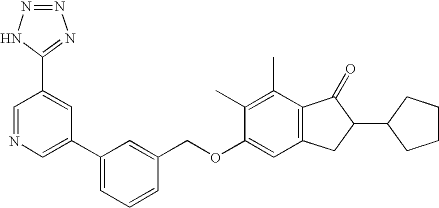

### 2-Cyclopentyl-6,7-dimethyl-5-({3-[5-(1H-tetrazol-5-yl)pyridin-3-yl]benzyl}oxy)indan-1-one

5-(3-{[(2-Cyclopentyl-6,7-dimethyl-1-oxo-2,3-dihydro-1H-inden-5-yl)oxy]methyl}phenyl)nicotinonitrile was synthesized as described in the general procedures A and E.

### General Procedure F: Tetrazole Formation

A mixture of 5-(3-{[(2-cyclopentyl-6,7-dimethyl-1-oxo-2,3-dihydro-1H-inden-5-yl)oxy]methyl}phenyl)nicotinonitrile (200 mg, 0.46 mmol), azido(trimethyl)silane (2 mL), and a catalytic amount of dibutyl(oxo)stannane in Toluene (10 mL) was refluxed for 24 h. The solvent was removed and the residue was purified by preparative HPLC to yield 78 mg of 2-cyclopentyl-6,7-dimethyl-5-({3-[5-(1H-tetrazol-5-yl)pyridin-3-yl]benzyl}oxy)indan-1-one as a yellow solid. ¹H NMR (DMSO-d₆, 500 MHz) δ9.22 (s, 1H), 9.12 (s, 1H), 8.68 (s, 1H), 7.97 (s, 1H), 7.83 (m, 1H), 7.63 (m, 2H), 7.09 (s, 1H), 5.32 (s, 2H), 3.11 (m, 1H), 2.67 (m, 2H), 2.53 (s, 3H), 2.20 (m, 10H), 2.14 (s, 3H), 1.82 (m, 1H), 1.58-1.32 (m, 6H), 1.05 (m, 1H). MS (ESI⁺) 480 (M⁺+1).

### EXAMPLE 30

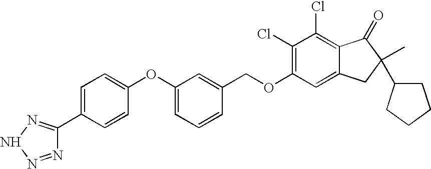

### 6,7-dichloro-2-cyclopentyl-2-methyl-5-(3-[4-(2H-tetrazol-5-yl)phenoxy]benzyl oxy)indan-1-one

### General Procedure G: O-Linked Biphenyl Formation

A mixture of 4-fluorobenzonitrile (8 g, 66 mmol), 3-hydroxymethylphenol (12.3 g, 99 mmol), and K₂CO₃ (18 g, 132 mmol) in DMF was heated to 110 C for 14 h. The reaction was cooled, diluted with EtOAc and washed with 1M NaOH (4×), brine, dried (MgSO₄) and concentrated. The resulting crude amber oil was used without further purification. The synthesis of the title compound was completed following General procedures B, C, and F using 6,7-dichloro-2-cyclopentyl-5-hydroxy-2-methylindan-1-one as a starting material. MS (ESI⁺) 549.14 (M⁺).

### EXAMPLE 31

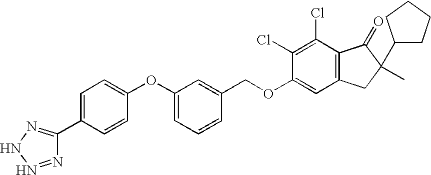

### 6-chloro-2-cyclopentyl-2-methyl-5-({3-[4-(2H-tetrazol-5-yl)phenoxy]benzyl}oxy)indan-1-one

A mixture of 6,7-dichloro-2-cyclopentyl-5-hydroxy-2-methylindan-1-one and Pd/C was stirred in MeOH under a balloon of H₂. After 24 h, the reaction mixture was filtered through a pad of celite and concentrated. The crude residue was purified by reverse phase preparative HPLC chromatography to give 6-chloro-2-cyclopentyl-5-hydroxy-2-methylindan-1-one as a colorless solid. The synthesis of the title compound was completed following general procedures G, B, C, and F using 4-fluorobenzonitrile and 6-chloro-2-cyclopentyl-5-hydroxy-2-methylindan-1-one as starting materials. MS (ESI⁺) 515.14 (M⁺).

### EXAMPLE 32

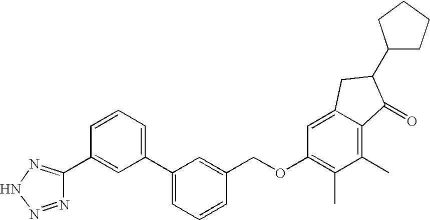

### 2-cyclopentyl-6,7-dimethyl-5-{[3′-(2H-tetrazol-5-yl)biphenyl-3-yl]methoxy}indan-1-one

2-cyclopentyl-6,7-dimethyl-5-{[3′-(2H-tetrazol-5-yl)biphenyl-3-yl]methoxy}indan-1-one was synthesized as described in general procedures A, B, C, and F using 3-bromobenzonitrile and 2-cyclopentyl-5-hydroxy-6,7-dimethylindan-1-one as starting materials. ¹H NMR (DMSO-d₆, 500 MHz) δ 8.36 (s, 1H), 8.05 (d, 1H), 7.92 (d, 1H), 7.88 (s, 1H), 7.7.1-7.76 (m, 2H), 7.55-7.60 (m, 2H), 7.09 (s, 1H), 5.28 (s, 2H), 3.0-3.2 (m, 1H), 2.64-2.70 (m, 2H), 2.53 (s, 3H), 2.18-2.20 (m, 1H), 2.14 (s, 3H), 1.82-1.84 (m, 1H), 1.45-1.58 (m, 5H), 1.31-1.36 (m, 1H), 1.01-1.03 (m, 1H). MS (ESI⁺) 479.96 (M⁺+1).

### EXAMPLE 33

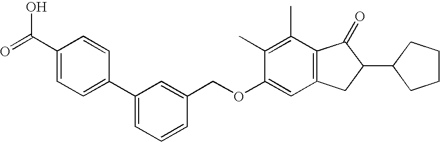

### 3′-{[(2-Cyclopentyl-6,7-dimethyl-1-oxo-2,3-dihydro-1H-inden-5-yl)oxy]methyl}biphenyl-4-carboxylic acid

3′-{([(2-Cyclopentyl-6,7-dimethyl-1-oxo-2,3-dihydro-1H-inden-5-yl)oxy]methyl}biphenyl-4-carboxylic acid was synthesized as described in general procedures A, B, C, and D using ethyl-4 iodobenzoate and 2-cyclopentyl-5-hydroxy-6,7-dimethylindan-1-one as starting materials. ¹H NMR (DMSO-d₆, 500 MHz) δ 13.00 (bs, 1H), 8.05 (d, 2H), 7.86 (s, 1H), 7.82 (d, 2H), 7.73 (d, 1H), 7.56 (m, 2H), 7.08 (s, 1H), 5.30 (s, 2H), 3.10 (m, 1H), 2.67 (m, 2H), 2.53 (s, 3H), 2.20 (m, 1H), 2.14 (s, 3H), 1.85 (m, 1H), 1.60-1.46 (m, 5H), 1.35 (m, 1H), 1.04 (m, 1H). MS (ESI⁺) 455 (M⁺+1).

### EXAMPLE 34

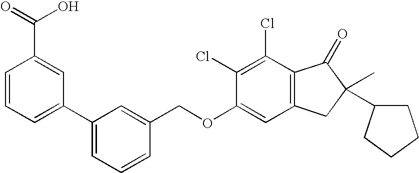

### 3′-{[(6,7-Dichloro-2-cyclopentyl-2-methyl-1-oxo-2,3-dihydro-1H-inden-5-yl)oxy]methyl}biphenyl-3-carboxylic acid

3′-{[6,7-Dichloro-2-cyclopentyl-2-methyl-1-oxo-2,3-dihydro-1H-inden-5-yl)oxy]methyl}biphenyl-3-carboxylic acid was synthesized as described in general procedures A, B, C, and D using as ethyl-3-bromobenzoate and 6,7-dichloro-2-cyclopentyl-5-hydroxy-2-methylindan-1-one starting materials. ¹H NMR (DMSO-d₆, 500 MHz) δ8.18 (s, 1H), 7.85 (d, 1H), 7.79 (s, 1H), 7.65 (d, 1H), 7.57 (d, 1H), 7.51 (t, 1H), 7.46 (m, 2H), 7.36 (t, 1H), 5.43 (s, 2H), 3.01 (d, 1H), 2.99 (d, 1H), 2.06 (m, 1H), 1.72 (m, 1H), 1.58-1.40 (m, 4H), 1.32 (m, 1H), 1.18 (m, 1H), 1.12 (s, 3H), 0.85 (m, 1H). MS (ESI⁺) 509 (M⁺+1).

### EXAMPLE 35

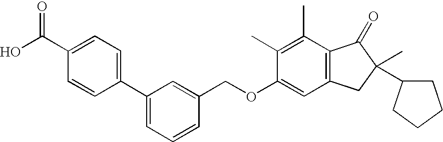

### 3′-{[(2-cyclopentyl-2,6,7-trimethyl-1-oxo-2,3-dihydro-1H-inden-5-yl)oxy]methyl}biphenyl-4-carboxylic acid

3′-{[(2-cyclopentyl-2,6,7-trimethyl-1-oxo-2,3-dihydro-1H-inden-5-yl)oxy]methyl}biphenyl-4-carboxylic acid was synthesized as described in general procedures A, B, C, and D using ethyl-4-bromobenzoate and 2-cyclopentyl-5-hydroxy-2,6,7-trimethylindan-1-one as starting materials. ¹H NMR (DMSO-d₆, 500 MHz) δ 13.0 (br s, 1H), 8.05 (d, 2H), 7.89 (br s, 1H), 7.83 (d, 2H), 7.73 (dt, 1H), 7.54-7.58 (m, 2H), 7.08 (s, 1H), 5.30 (s, 2H), 2.92 (d, 1H), 2.65 (d, 1H), 2.51 (s, 3H), 2.14 (s, 3H), 2.05-2.09 (m, 1H), 1.74-1.76 (m, 1H), 1.38-1.54 (m, 4H), 1.29-1.35 (m, 1H), 1.18-1.28 (m, 1H), 1.11 (s, 3H), 0.80-0.85 (m, 1H). MS (ESI⁺) 470.15 (M⁺+1).

### EXAMPLE 36

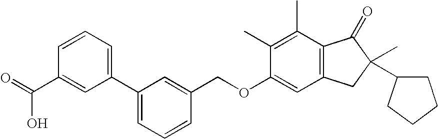

### 3′-{[(2-cyclopentyl-2,6,7-trimethyl-1-oxo-2,3-dihydro-1H-inden-5-yl)oxy]methyl}biphenyl-3-carboxylic acid

3′-{[(2-cyclopentyl-2,6,7-trimethyl-1-oxo-2,3-dihydro-1H-inden-5-yl)oxy]methyl}biphenyl-3-carboxylic acid was synthesized as described in general procedures A, B, C, and D using ethyl-3-iodobenzoate and 2-cyclopentyl-5-hydroxy-2,6,7-trimethylindan-1-one as starting materials. ¹H NMR (DMSO-d₆, 500 MHz) δ 13.0 (br s, 1H), 8.23 (t, 1H), 7.94-7.98 (m, 2H), 7.84 (s, 1H), 7.70 (dt, 1H), 7.63 (t, 1H), 7.53-7.56 (m, 2H), 7.08 (s, 1H), 5.31 (s, 2H), 2.90 (d, 1H), 2.65 (d, 1H), 2.54 (s, 3H), 2.09 (s, 3H), 2.05-2.09 (m, 1H), 1.73-1.76 (m, 1H), 1.38-1.54 (m, 4H), 1.29-1.35 (m, 1H), 1.18-1.28 (m, 1H), 1.09 (s, 3H), 0.82-0.84 (m, 1H). MS (ESI⁺) 470.12 (M⁺+1).

### EXAMPLE 37

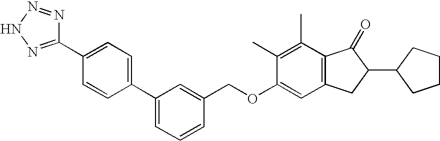

### 2-cyclopentyl-6,7-dimethyl-5-{[4′-(2H-tetrazol-5-yl)biphenyl-3-yl]methoxy}indan-1-one

2-cyclopentyl-6,7-dimethyl-5-{[4′-(2H-tetrazol-5-yl)biphenyl-3-yl]methoxy}indan-1-one was synthesized as described in general procedures A, B, C, and F using 4-bromobenzonitrile and 2-cyclopentyl-5-hydroxy-6,7-dimethylindan-1-one as starting materials. ¹H NMR (DMSO-d₆, 500 MHz) δ 8.16 (d, 2H), 7.95 (d, 2H), 7.89 (s, 1H), 7.76 (dt, 1H), 7.52-7.59 (m, 2H), 7.09 (s, 1H), 5.36 (s, 2H), 3.07-3.17 (m, 1H), 2.64-2.71 (m, 2H), 2.51 (s, 3H), 2.17-2.21 (m, 1H), 2.15 (s, 3H), 1.84-1.85 (m, 1H), 1.46-1.60 (m, 5H), 1.34-1.40 (m, 1H), 1.03-1.05 (m, 1H). MS (ESI⁺) 480.15 (M⁺+1).

### EXAMPLE 38

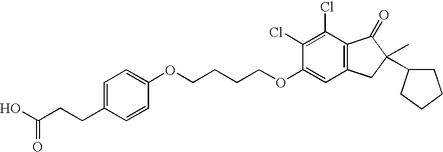

### 3-(4-{4-[(6,7-dichloro-2-cyclopentyl-2-methyl-1-oxo-2,3-dihydro-1H-inden-5-yl)oxy]butoxy}phenyl)propanoic acid

A mixture of 6,7-dichloro-2-cyclopentyl-5-hydroxy-2-methylindan-1-one (100 mg, 0.33 mmol), methyl 3-[4-(4-bromobutoxy)phenyl]propanoate (105 mg, 0.33 mmol), potassium carbonate (136 mg, 0.99 mmol) and acetone (3.3 ml) was heated overnight at 45° C. The reaction mixture was cooled and acidified to pH 1 with 1.0 N HCl aqueous solution, then extracted with ethyl acetate. The combined organic extracts were dried over sodium sulfate, filtered and concentrated. The resulting crude oil was purified by flash chromatography on silica gel (0-20% ethyl acetate/hexanes) to give methyl 3-(4-{4-[(6,7-dichloro-2-cyclopentyl-2-methyl-1-oxo-2,3-dihydro-1H-inden-5-yl)oxy]butoxy}phenyl)propanoate (118 mg). A mixture of methyl 3-(4-{4-[(6,7-dichloro-2-cyclopentyl-2-methyl-1-oxo-2,3-dihydro-1H-inden-5-yl)oxy]butoxy}phenyl)propanoate (105 mg, 0.2 mmol), tetrahydrofuran (4.0 ml) and 2.0 N sodium hydroxide aqueous solution (4.0 ml) was stirred at room temperature until no starting material was observed by tlc. The reaction mixture was acidified to pH 1 with 1.0 N HCl aqueous solution and extracted with ethyl acetate. The combined organic extracts were dried over sodium sulfate, filtered and concentrated. Flash chromatography on silica gel (0-100% ethyl acetate/hexanes) of the crude material afforded the desired product as a white solid. ¹H NMR (CDCl₃, 300 MHz) δ 7.13 (d, 2 μl), 6.85 (m, 3H), 4.20 (t, 2H), 4.08 (t, 2H), 3.00-2.97 (d, 1H), 2.92 (t, 1H), 2.71-2.65 (m, 2H), 2.12 (m, 1H), 2.12-2.03 (m, 4H), 1.86-1.83 (m, 1H), 1.61-1.50 (m, 5H), 1.24-1.22 (m, 1H), 1.23 (s, 3H), 0.90 (m, 1H).

MS (ESI) 541 (M⁺+Na).

### EXAMPLE 39

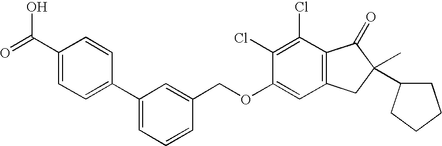

### 3′-{[(6,7-Dichloro-2-cyclopentyl-2-methyl-1-oxo-2,3-dihydro-1H-inden-5-yl)oxy]methyl}biphenyl-4-carboxylic acid

3′-{[(6,7-Dichloro-2-cyclopentyl-2-methyl-1-oxo-2,3-dihydro-1H-inden-5-yl)oxy]methyl}biphenyl-4-carboxylic acid was synthesized as described in general procedures A, B, C, and D using as ethyl-4-bromobenzoate and 6,7-dichloro-2-cyclopentyl-5-hydroxy-2-methylindan-1-one starting materials. ¹H NMR (DMSO-d₆, 500 MHz) δ 7.95 (d, 2H), 7.81 (s, 1H), 7.68 (d, 1H), 7.58 (d, 2H), 7.52 (t, 1H), 7.49 (d, 2H), 5.42 (s, 2H), 3.00 (d, 1H), 2.77 (d, 1H), 2.07 (m, 1H), 1.75 (m, 1H), 1.55-1.42 (m, 4H), 1.33 (m, 1H), 1.19 (m, 1H), 1.14 (s, 3H), 0.85 (m, 1H). MS (ESI⁺) 509 (M⁺+1).

### EXAMPLE 40

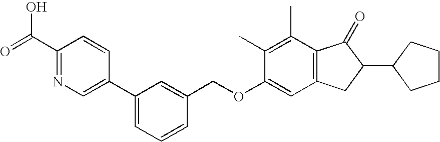

### 5-(3-{[(2-Cyclopentyl-6,7-dimethyl-1-oxo-2,3-dihydro-1H-inden-5-yl)oxy]methyl}phenyl)pyridine-2-carboxylic acid

5-(3-{[(2-Cyclopentyl-6,7-dimethyl-1-oxo-2,3-dihydro-1H-inden-5-yl)oxy]methyl}phenyl)pyridine-2-carboxylic acid was synthesized as described in general procedures A, E, and D using ethyl 5-bromopyridine-2-carboxylate and 2-cyclopentyl-5-hydroxy-6,7-dimethylindan-1-one as starting materials. ¹H NMR (DMSO-d₆, 500 MHz) δ 9.04 (s, 1H), 8.28 (d, 1H), 8.14 (d, 1H), 7.93 (s, 1H), 7.80 (m, 1H), 7.59 (d, 2H), 7.08 (s, 1H), 5.30 (s, 2H), 3.08 (m, 1H), 2.66 (m, 2H), 2.52 (s, 3H), 2.20 (m, 1H), 2.13 (s, 3H), 1.83 (m, 1H), 1.59-1.31 (m, 6H), 1.02 (m, 1H).

### EXAMPLE 41

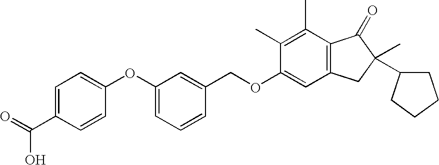

### 4-(3-{[(2-cyclopentyl-2,6,7-trimethyl-1-oxo-2,3-dihydro-1H-inden-5-yl)oxy]methyl}phenoxy)benzoic acid

4-(3-{[(2-cyclopentyl-2,6,7-trimethyl-1-oxo-2,3-dihydro-1H-inden-5-yl)oxy]methyl}phenoxy)benzoic acid was synthesized following general procedures G, B, C, and D using 4-fluoromethylbenzoate and 2-cyclopentyl-5-hydroxy-2,6,7-trimethylindan-1-one as starting materials. MS (ESI⁺) 526.85 (M⁺+1).

### EXAMPLE 42

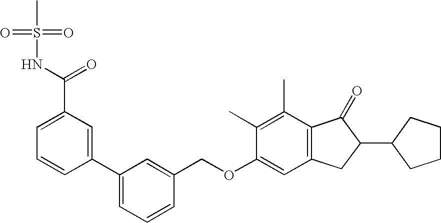

### 3′-{[(2-cyclopentyl-6,7-dimethyl-1-oxo-2,3-dihydro-1H-inden-5-yl)oxy]methyl}-N-(methylsulfonyl)biphenyl-3-carboxamide

To a stirred solution of 3′-{[(2-cyclopentyl-6,7-dimethyl-1-oxo-2,3-dihydro-1H-inden-5-yl)oxy]methyl}biphenyl-3-carboxylic acid (200 mg, 0.44 mmol) in dichloromethane (10 ml) at rt was added oxalyl chloride (0.19 ml, 2.2 mmol) followed by a few drops of dimethylformamide (0.05 ml). After stirring at rt for 4 h, the reaction was concentrated in vacuo to give the acid chloride as a yellow solid. The acid chloride was dissolved in tetrahydrofuran (10 ml) and added in one portion to a cooled stirred solution of sodium hydride (176 mg, 4.4 mmol), methane sulfonamide (418 mg, 4.4 mmol) and tetrahydrofuran (5.0 ml) at 0° C. The reaction mixture was allowed to warm to rt overnight and then quenched with 1M HCl and diluted with ethyl acetate. The layers were separated and the organic phase was washed with brine (2×30 ml), dried (MgSO4) and concentrated. The crude residue was purified by reverse phase preparative HPLC chromatography to give the title compound as a colorless solid. ¹H NMR (CDCl₃, 500 MHz) δ 9.08 (s, 1H), 8.13 (s, 1H), 7.86 (t, 1H), 7.75 (s, 1H), 7.55-7.64 (m, 2H), 7.51-7.54 (m, 2H), 6.94 (s, 1H), 5.15 (s, 2H), 3.46 (s, 3H), 3.22-3.32 (m, 3H), 2.67 (s, 3H), 2.29 (s, 3H), 1.95-2.05 (m, 2H), 1.65-1.82 (m, 5H), 1.48-1.52 (m, 2H).

### EXAMPLE 43

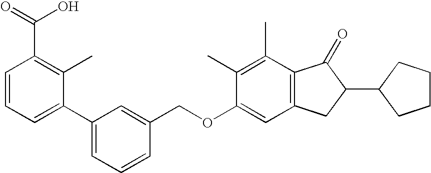

### 3′-{[(2-Cyclopentyl-6,7-dimethyl-1-oxo-2,3-dihydro-1H-inden-5-yl)oxy]methyl}-2-methylbiphenyl-3-carboxylic acid

3′-{[(2-Cyclopentyl-6,7-dimethyl-1-oxo-2,3-dihydro-1H-inden-5-yl)oxy]methyl}-2-methylbiphenyl-3-carboxylic acid was synthesized as described in general procedures A, B, C, and D using methyl 3-bromo-2-methylbenzoate and 2-cyclopentyl-5-hydroxy-6,7-dimethylindan-1-one as starting materials. ¹H NMR (CDCl₃, 500 MHz) δ 8.02 (d, 1H), 7.45 (m, 3H), 7.37 (s, 1H), 7.33 (t, 1H), 7.28 (d, 1H), 6.79 (s, 1H), 5.19 (s, 2H), 3.08 (m, 1H), 2.71 (m, 2H), 2.62 (s, 3H), 2.50 (s, 3H), 2.33 (m, 1H), 2.20 (s, 3H), 1.91 (m, 1H), 1.65-1.59 (m, 5H), 1.40 (m, 1H), 1.06 (m, 1H). MS (ESI⁺) 469 (M⁺+1).

### EXAMPLE 44

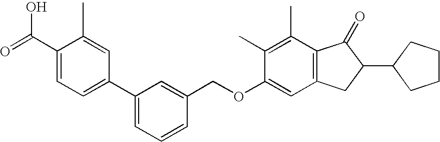

### 3′-{[(2-Cyclopentyl-6,7-dimethyl-3-oxo-2,3-dihydro-H-inden-5-yl)oxy]methyl}-3-methylbiphenyl-4-carboxylic acid

3′-{[(2-Cyclopentyl-6,7-dimethyl-1-oxo-2,3-dihydro-1H-inden-5-yl)oxy]methyl}-3-methylbiphenyl-4-carboxylic acid was synthesized as described in general procedures A, B, C, and D using methyl 4-bromo-2-methylbenzoate and 2-cyclopentyl-5-hydroxy-6,7-dimethylindan-1-one as starting materials. ¹H NMR (CDCl₃, 500 MHz) δ 8.17 (d, 1H), 7.70 (s, 1H), 7.61 (d, 1H), 7.51 (m, 4H), 6.80 (s, 1H), 5.21 (s, 2H), 3.09 (m, 1H), 2.76-2.69 (m, 5H), 2.63 (s, 3H), 2.32 (m, 1H), 2.22 (s, 3H), 1.93 (m, 1H), 1.65-1.51 (m, 5H), 1.40 (m, 1H), 1.06 (m, 1H). MS (ESI⁺) 469 (M⁺+1).

### EXAMPLE 45

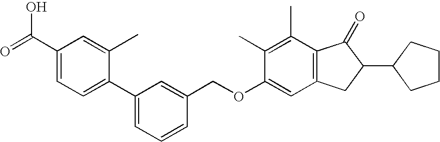

### 3′-{[(2-Cyclopentyl-6,7-dimethyl-1-oxo-2,3-dihydro-1H-inden-5-yl)oxy]methyl}-2-methylbiphenyl-4-carboxylic acid

3′-{[(2-Cyclopentyl-6,7-dimethyl-1-oxo-2,3-dihydro-1H-inden-5-yl)oxy]methyl}-2-methylbiphenyl-4-carboxylic acid was synthesized as described in general procedures A, B, C, and D using methyl 4-bromo-3-methylbenzoate and 2-cyclopentyl-5-hydroxy-6,7-dimethylindan-1-one as starting materials. ¹H NMR (CDCl₃, 500 MHz) δ 8.04 (s, 1H), 7.99 (d, 1H), 7.49 (m, 2H), 7.41 (s, 1H), 7.35 (d, 1H), 7.32 (d, 1H), 6.79 (s, 1H), 5.20 (s, 2H), 3.06 (m, 1H), 2.71 (m, 2H), 2.62 (s, 3H), 2.33 (m, 4H), 2.20 (s, 3H), 1.91 (m, 1H), 1.64-1.51 (m, 5H), 1.39 (m, 1H), 1.06 (m, 1H). MS (ESI⁺) 469 (M⁺+1).

### EXAMPLE 46

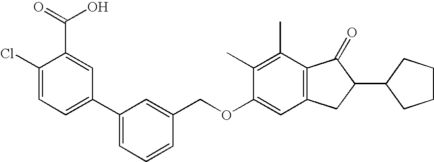

### 4-Chloro-3′-{[(2-cyclopentyl-6,7-dimethyl-1-oxo-2,3-dihydro-1H-inden-5-yl)oxy]methyl}biphenyl-3-carboxylic acid

4-Chloro-3′-{[(2-cyclopentyl-6,7-dimethyl-1-oxo-2,3-dihydro-1H-inden-5-yl)oxy]methyl}biphenyl-3-carboxylic acid was synthesized as described in general procedures A, B, C, and D using methyl 5-bromo-2-chlorobenzoate and 2-cyclopentyl-5-hydroxy-6,7-dimethylindan-1-one as starting materials. ¹H NMR (CDCl₃, 500 MHz) δ 8.25 (s, 1H), 7.70 (d, 1H), 7.66 (s, 1H), 7.56 (m, 2H), 7.50 (m, 2H), 6.79 (s, 1H), 5.20 (s, 2H), 3.07 (m, 1H), 2.72 (m, 2H), 2.62 (s, 3H), 2.33 (m, 1H), 2.22 (s, 3H), 1.92 (m, 1H), 1.64-1.51 (m, 5H), 1.39 (m, 1H), 1.06 (m, 1H). MS (ESI⁺) 489 (M⁺).

### EXAMPLE 47

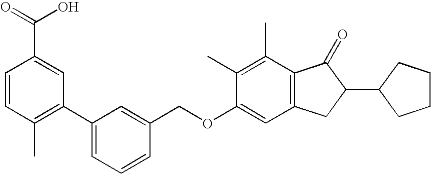

### 3′-{([(2-Cyclopentyl-6,7-dimethyl-1-oxo-2,3-dihydro-1H-inden-5-yl)oxy]methyl}-6-methylbiphenyl-3-carboxylic acid

3′-{[(2-Cyclopentyl-6,7-dimethyl-1-oxo-2,3-dihydro-1H-inden-5-yl)oxy]methyl}-6-methylbiphenyl-3-carboxylic acid was synthesized as described in general procedures A, B, C, and D using methyl 3-bromo-4-methylbenzoate and 2-cyclopentyl-5-hydroxy-6,7-dimethylindan-1-one as starting materials. ¹H NMR (CDCl₃, 500 MHz) δ 8.00 (m, 2H), 7.48 (m, 2H), 7.39 (m, 2H), 7.32 (d, 1H), 6.79 (s, 1H), 5.19 (s, 2H), 3.05 (m, 1H), 2.71 (m, 2H), 2.62 (s, 3H), 2.33 (s, 3H), 2.20 (s, 3H), 1.92 (m, 1H), 1.65-1.51 (m, 6H), 1.39 (m, 1H), 1.06 (m, 1H). MS (ESI⁺) 469 (M⁺+1).

### EXAMPLE 48

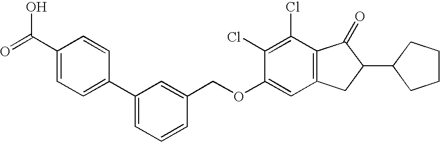

### 3′-{[(6,7-Dichloro-2-cyclopentyl-1-oxo-2,3-dihydro-1H-inden-5-yl)oxy]methyl}biphenyl 4-carboxylic acid

3′-{([(6,7-Dichloro-2-cyclopentyl-1-oxo-2,3-dihydro-1H-inden-5-yl)oxy]methyl}biphenyl-4-carboxylic acid was synthesized as described in general procedures A, B, C, and D using ethyl-4 iodobenzoate and 6,7-dichloro-2-cyclopentyl-5-hydroxyindan-1-one as starting materials. ¹H NMR (DMSO-d₆, 500 MHz) δ 8.05 (d, 2H), 7.88 (s, 1H), 7.82 (d, 2H), 7.76 (d, 1H), 7.56 (m, 2H), 7.47 (s, 1H), 5.76 (s, 2H), 3.20 (m, 1H), 2.77 (m, 2H), 2.20 (m, 1H), 1.85 (m, 1H), 1.61-1.47 (m, 5H), 1.35 (m, 1H), 1.10 (m, 1H). MS (ESI⁺) 495 (M⁺).

### EXAMPLE 49

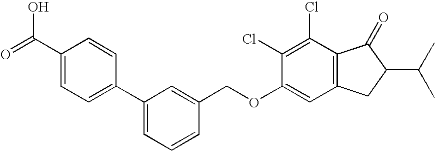

### 3′-{([(6,7-Dichloro-2-isopropyl-1-oxo-2,3-dihydro-1H-inden-5-yl)oxy]methyl}biphenyl-4-carboxylic acid

3′-{([(6,7-Dichloro-2-isopropyl-1-oxo-2,3-dihydro-1H-inden-5-yl)oxy]methyl}biphenyl-4-carboxylic acid was synthesized as described in general procedures A, B, C, and D using ethyl-4 iodobenzoate and 6,7-dichloro-5-hydroxy-2-isopropylindan-1-one as starting materials. ¹H NMR (DMSO-d₆, 500 MHz) δ 13.07 (bs, 1H), 8.12 (d, 2H), 7.97 (s, 1H), 7.91 (d, 2H), 7.84 (m, 1H), 7.65 (m, 2H), 7.58 (s, 10H), 5.52 (s, 2H), 3.19 (m, 1H), 2.93 (d, 1H), 2.82 (m, 1H), 2.31 (m, 1H), 1.06 (d, 3H), 0.81 (d, 3H). MS (ESI⁺) 469 (M⁺).

### EXAMPLE 50

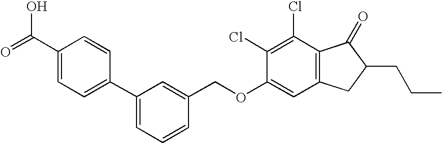

### 3′-{[(6,7-Dichloro-1-oxo-2-propyl-2,3-dihydro-1H-inden-5-yl)oxy]methyl}biphenyl-4-carboxylic acid

3′-{[(6,7-Dichloro-1-oxo-2-propyl-2,3-dihydro-1H-inden-5-yl)oxy]methyl}biphenyl-4-carboxylic acid was synthesized as described in general procedures A, B, C, and D using ethyl-4 iodobenzoate and 6,7-dichloro-5-hydroxy-2-propylindan-1-one as starting materials. ¹H NMR (DMSO-d₆, 500 MHz) δ 12.99 (bs, 1H), 8.05 (d, 2H), 7.88 (s, 1H), 7.82 (d, 2H), 7.75 (m, 1H), 7.58 (m, 2H), 7.48 (s, 1H), 5.45 (s, 2H), 3.26 (m, 2H), 2.72 (m, 2H), 1.75 (m, 1H), 1.38 (m, 2H), 0.89 (t, 3H). MS (ESI⁺) 469 (M⁺).

### EXAMPLE 51

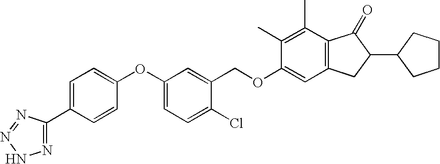

### 5-({2-chloro-5-[4-(2H-tetrazol-5-yl)phenoxy]benzyl}oxy)-2-cyclopentyl-6,7-dimethylindan-1-one

4-chloro-3-(hydroxymethyl)phenol was made by borane reduction of the corresponding acid. The synthesis of the title compound was then completed following general procedures G, B, C, and F using 4-chloro-3-(hydroxymethyl)phenol and 2-cyclopentyl-5-hydroxy-6,7-dimethylindan-1-one as starting materials. MS (ESI⁺) 528.99 (M⁺).

### EXAMPLE 52

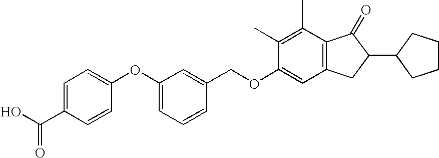

### 4-(3-{[(2-cyclopentyl-6,7-dimethyl-1-oxo-2,3-dihydro-1H-inden-5-yl)oxy]methyl}phenoxy)benzoic acid

4-(3-{[(2-cyclopentyl-6,7-dimethyl-1-oxo-2,3-dihydro-1H-inden-5-yl)oxy]methyl}-phenoxy)benzoic acid was synthesized following general procedures G, B, C, and D using 4-fluoromethylbenzoate and 2-cyclopentyl-5-hydroxy-6,7-dimethylindan-1-one as starting materials. MS (ESI⁺) 471.03 (M⁺+1).

### EXAMPLE 53

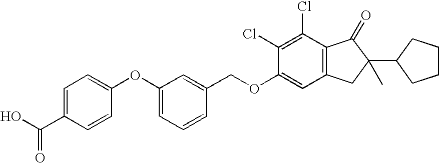

### 4-(3-{[(6,7-dichloro-2-cyclopentyl-2-methyl-1-oxo-2,3-dihydro-1H-inden-5-yl)oxy]methyl}phenoxy)benzoic acid

4-(3-{[(6,7-dichloro-2-cyclopentyl-2-methyl-1-oxo-2,3-dihydro-1H-inden-5-yl)oxy]methyl}phenoxy)-benzoic acid was synthesized following general procedures G, B, C, and D using 4-fluoromethylbenzoate and 6,7-dichloro-2-cyclopentyl-5-hydroxy-2-methylindan-1-one as starting materials MS (ESI⁺) 485.05 (M⁺+1).

### EXAMPLE 54

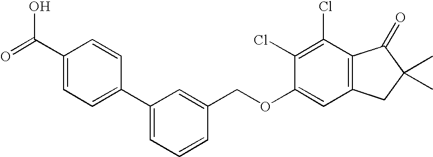

### 3′-{[(6,7-Dichloro-2,2-dimethyl-1-oxo-2,3-dihydro-1H-inden-5-yl)oxy]methyl}biphenyl-4-carboxylic acid

3′-{[(6,7-Dichloro-2,2-dimethyl-1-oxo-2,3-dihydro-1H-inden-5-yl)oxy]methyl}biphenyl-4-carboxylic acid was synthesized as described in general procedures A, B, C, and D using ethyl-4 iodobenzoate and 6,7-dichloro-5-hydroxy-2,2-dimethylindan-1-one as starting materials. ¹H NMR (DMSO-d₆, 500 MHz) δ 12.99 (bs, 1H), 8.05 (d, 2H), 7.89 (s, 1H), 7.83 (d, 2H), 7.76 (d, 1H), 7.58 (m, 2H), 7.49 (s, 1H), 5.45 (s, 2H), 2.97 (s, 2H), 1.14 (s, 6H).

### EXAMPLE 55

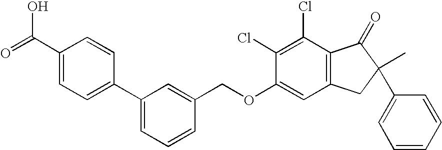

### 3′-{[(6,7-Dichloro-2-methyl-1-oxo-2-phenyl-2,3-dihydro-1H-inden-5-yl)oxy]methyl}biphenyl-4-carboxylic acid

3′-{[(6,7-Dichloro-2-methyl-1-oxo-2-phenyl-2,3-dihydro-1H-inden-5-yl)oxy]methyl}biphenyl-4-carboxylic acid was synthesized as described in general procedures A, B, C, and D using ethyl-4 iodobenzoate and 6,7-dichloro-5-hydroxy-2-methyl-2-phenylindan-1-one as starting materials. ¹H NMR (DMSO-d₆, 500 MHz) δ 12.99 (bs, 1H), 8.06 (d, 2H), 7.90 (s, 1H), 7.83 (d, 2H), 7.76 (m, 1H), 7.58 (m, 3H), 7.30 (m, 2H), 7.26 (m, 3H), 5.47 (s, 2H), 3.52 (d, 1H), 3.32 (d, 1H), 1.57 (s, 3H). MS (ESI⁺) 517 (M⁺+1).

### EXAMPLE 56

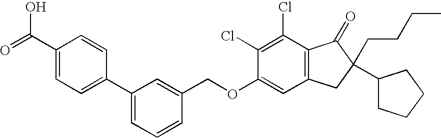

### 3′-{[(2-Butyl-6,7-dichloro-2-cyclopentyl-1-oxo-2,3-dihydro-1H-inden-5-yl)oxy]methyl}biphenyl-4-carboxylic acid

3′-{[(2-Butyl-6,7-dichloro-2-cyclopentyl-1-oxo-2,3-dihydro-1H-inden-5-yl)oxy]methyl}biphenyl-4-carboxylic acid was synthesized as described in general procedures A, B, C, and D using ethyl-4 iodobenzoate and 2-butyl-6,7-dichloro-2-cyclopentyl-5-hydroxyindan-1-one as starting materials. ¹H NMR (DMSO-d₆, 500 MHz) δ 13.03 (bs, 1H), 8.10 (d, 2H), 7.93 (s, 1H), 7.86 (d, 2H), 7.80 (m, 1H), 7.62 (m, 2H), 7.53 (s, 1H), 5.48 (s, 2H), 3.36 (s, 2H), 2.95 (m, 2H), 2.16 (m, 1H), 1.77 (m, 1H), 1.66-1.35 (m, 6H), 1.22 (m, 3H), 0.90 (m, 2H), 0.82 (t, 3H). MS (ESI⁺) 551 (M⁺).

### EXAMPLE 57

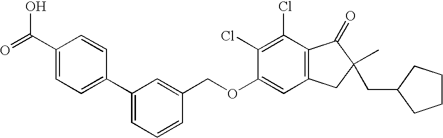

### 3′-([6,7-Dichloro-2-(cyclopentylmethyl)-2-methyl-1-oxo-2,3-dihydro-1H-inden-5-yl]oxy)methyl)biphenyl-4-carboxylic acid

3′-({[6,7-Dichloro-2-(cyclopentyl methyl)-2-methyl-1-oxo-2,3-dihydro-1H-inden-5-yl]oxy}methyl)biphenyl-4-carboxylic acid was synthesized as described in general procedures A, B, C, and D using ethyl-4 iodobenzoate and 6,7-dichloro-2-(cyclopentylmethyl)-5-hydroxy-2-methylindan-1-one as starting materials. ¹H NMR (DMSO-d₆, 500 MHz) δ 12.77 (bs, 1H), 7.83 (d, 2H), 7.68 (s, 1H), 7.62 (d, 2H), 7.55 (d, 1H), 7.37 (m, 2H), 7.28 (s, 1H), 5.24 (s, 2H), 2.92 (d, 1H), 2.66 (d, 1H), 1.49-1.12 (m, 9H), 0.91 (s, 3H), 0.83 (m, 1H), 0.74 (m, 1H).

### EXAMPLE 58

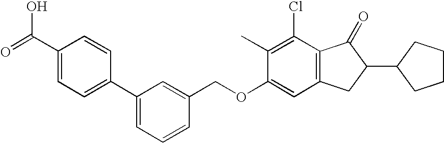

### 3′-{[(7-Chloro-2-cyclopentyl-6-methyl-1-oxo-2,3-dihydro-1H-inden-5-yl)oxy]methyl}biphenyl-4-carboxylic acid

3′-{[(7-Chloro-2-cyclopentyl-6-methyl-1-oxo-2,3-dihydro-1-yl)oxy]methyl}biphenyl-4-carboxylic acid was synthesized as described in general procedures A, B, C, and D using ethyl-4 iodobenzoate and 7-chloro-2-cyclopentyl-5-hydroxy-6-methylindan-1-one as starting materials. ¹H NMR (DMSO-d₆, 500 MHz) δ 12.99 (bs, 1H), 8.04 (d. 2H), 7.86 (s, 1H), 7.82 (d, 2H), 7.74 (m, 1H), 7.56 (m, 2H), 7.25 (s, 1H), 5.35 (s, 2H), 3.14 (m, 1H), 2.73 (m, 2H), 2.26 (s, 3H), 2.19 (m, 1H), 1.84 (m, 1H), 1.60-1.46 (m, 5H), 1.34 (m, 1H), 1.05 (m, 1H).

### EXAMPLE 59

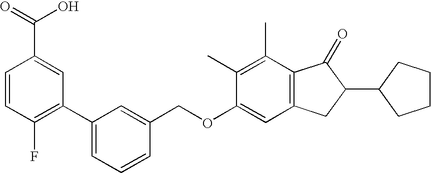

### 3′-{[(2-Cyclopentyl-6,7-dimethyl-1-oxo-2,3-dihydro-1H-inden-5-yl)oxy]methyl}-6-fluorobiphenyl-3-carboxylic acid

2-Cyclopentyl-5-{[3-(5,5-dimethyl-1,3,2-dioxaborinan-2-yl)benzyl]oxy}-6,7-dimethylindan-1-one was synthesized following general procedure C using 2-[3-(bromomethyl)phenyl]-5,5-dimethyl-1,3,2-dioxaborinane and cyclopentyl-5-hydroxy-6,7-dimethylindan-1-one as starting materials. Methyl 3′-{[(2-cyclopentyl-6,7-dimethyl-1-oxo-2,3-dihydro-1H-inden-5-yl)oxy]methyl}-6-fluorobiphenyl-3-carboxylate was synthesized following general procedure A using 2-cyclopentyl-5-{[3-(5,5-dimethyl-1,3,2-dioxaborinan-2-yl)benzyl]oxy}-6,7-dimethylindan-1-one and methyl 3-bromo-4-fluorobenzoate as starting materials. 3′-{[(2-Cyclopentyl-6,7-dimethyl-1-oxo-2,3-dihydro-1H-inden-5-yl)oxy]methyl}-6-fluorobiphenyl-3-carboxylic acid was synthesized from methyl 3′-{[(2-cyclopentyl-6,7-dimethyl-1-oxo-2,3-dihydro-1H-inden-5-yl)oxy]methyl}-6-fluorobiphenyl-3-carboxylate following general procedure D. ¹H NMR (DMSO-d₆, 500 MHz) δ 13.15 (bs, 1H), 8.06 (d, 1H), 7.99 (m, 1H), 7.70 (s, 1H), 7.55 (m, 3H), 7.45 (t, 1H), 7.06 (s, 1H), 5.29 (s, 2H), 3.10 (m, 1H), 2.65 (m, 2H), 2.51 (s, 3H), 2.18 (m, 1H), 2.12 (s, 3H), 1.82 (m, 1H), 1.58-1.44 (m, 5H), 1.32 (m, 1H), 1.02 (m, 1H). MS (ESI⁺) 473 (M⁺+1).

### EXAMPLE 60

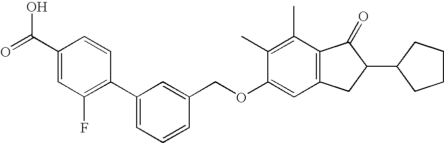

### 3′-{[(2-Cyclopentyl-6,7-dimethyl-1-oxo-2,3-dihydro-1H-inden-5-yl)oxy]methyl}-2-fluorobiphenyl-4-carboxylic acid

3′-{[(2-Cyclopentyl-6,7-dimethyl-1-oxo-2,3-dihydro-1H-inden-5-yl)oxy]methyl}-2-fluorobiphenyl-4-carboxylic acid was synthesized as described in general procedures A and D using 2-cyclopentyl-5-{[3-(5,5-dimethyl-1,3,2-dioxaborinan-2-yl)benzyl]oxy}-6,7-dimethylindan-1-one and methyl 4-bromo-3-fluorobenzoate as starting materials. ¹H NMR (DMSO-d₆, 500 MHz) δ 13.32 (bs, 1H), 7.87 (d, 1H), 7.80 (d, 1H), 7.74 (s, 1H), 7.70 (t, 1H), 7.59 (m, 3H), 7.08 (s, 1H), 3.09 (m, 1H), 2.65 (m, 2H), 2.53 (s, 3H), 2.19 (m, 1H), 2.14 (s, 3H), 1.84 (m, 1H), 1.60-1.46 (m, 5H), 1.35 (m, 1H), 1.04 (m, 1H). MS (ESI⁺) 473 (M⁺+1).

### EXAMPLE 61

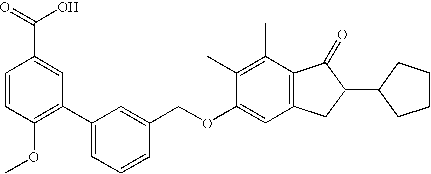

### 3′-{[(2-Cyclopentyl-6,7-dimethyl-1-oxo-2,3-dihydro-1H-inden-5-yl)oxy]methyl}-6-methoxybiphenyl-3-carboxylic acid

3′-{([(2-Cyclopentyl-6,7-dimethyl-1-oxo-2,3-dihydro-1H-inden-5-yl)oxy]methyl}-6-methoxybiphenyl-3-carboxylic acid was synthesized as described in general procedures A and D using 2-cyclopentyl-5-[3-(5,5-dimethyl-1,3,2-dioxaborinan-2-yl)benzyl]oxy)-6,7-dimethylindan-1-one and methyl 3-bromo-4-methoxybenzoate as starting material. ¹H NMR (DMSO-d₆, 500 MHz) δ 12.73 (bs, 1H), 7.96 (d, 1H), 7.86 (d, 1H), 7.62 (s, 1H), 7.48 (m, 3H), 7.24 (d, 1H), 7.08 (s, 1H), 5.28 (s, 2H), 3.85 (s, 3H), 3.10 (m, 1H), 2.67 (m, 2H), 2.53 (s, 3H), 2.20 (m, 1H), 2.14 (s, 3H), 1.85 (m, 1H), 1.61-1.35 (m, 6H), 1.04 (m, 1H). MS (ESI⁺) 485 (M⁺+1).

### EXAMPLE 62

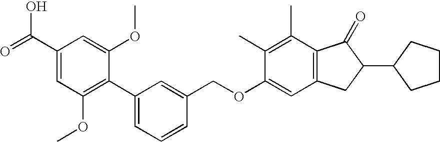

### 3′-{[(2-Cyclopentyl-6,7-dimethyl-1-oxo-2,3-dihydro-1H-inden-5-yl)oxy]methyl}-2,6-dimethoxybiphenyl-4-carboxylic acid

3′-{[(2-Cyclopentyl-6,7-dimethyl-1-oxo-2,3-dihydro-1H-inden-5-yl)oxy]methyl}-2,6-dimethoxybiphenyl-4-carboxylic acid was synthesized as described in general procedures A and D using 2-cyclopentyl-5-{[3-(5,5-dimethyl-1,3,2-dioxaborinan-2-yl)benzyl]oxy}-6,7-dimethylindan-1-one and methyl 4-bromo-3,5-dimethoxybenzoate as starting material. ¹H NMR (DMSO-d₆, 500 MHz) δ 13.03 (bs, 1H), 7.35 (m, 2H), 7.27 (s, 1H), 7.22 (s, 2H), 7.14 (m, 1H), 6.98 (s, 1H), 5.15 (s, 2H), 3.64 (s, 6H), 2.99 (m, 1H), 2.57 (m, 2H), 2.44 (s, 3H), 2.11 (m, 1H), 2.04 (s, 3H), 1.75 (m, 1H), 1.51-1.27 (m, 6H), 0.96 (m, 1H). MS (ESI⁺) 515 (M⁺+1).

### EXAMPLE 63

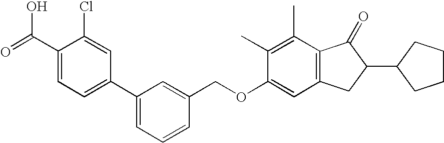

### 3-Chloro-3′-{[(2-cyclopentyl-6,7-dimethyl-1-oxo-2,3-dihydro-1H-inden-5-yl)oxy]methyl}biphenyl-4-carboxylic acid

3-Chloro-3′-{[(2-cyclopentyl-6,7-dimethyl-1-oxo-2,3-dihydro-1H-inden-5-yl)oxy]methyl}biphenyl-4-carboxylic acid was synthesized as described in general procedures A and D using 2-cyclopentyl-5-{[3-(5,5-dimethyl-1,3,2-dioxaborinan-2-yl)benzyl]oxy}-6,7-dimethylindan-1-one and methyl 4-bromo-2-chlorobenzoate as starting material. ¹H NMR (DMSO-d₆, 500 MHz) δ 13.40 (bs, 1H), 7.92 (d, 1H), 7.88 (s, 1H), 7.86 (s, 1H), 7.75 (m, 2H), 7.56 (m, 2H), 7.08 (s, 1H), 5.30 (s, 2H), 3.09 (m, 1H), 2.66 (m, 2H), 2.53 (s, 3H), 2.20 (m, 1H), 2.14 (s, 3H), 1.84 (m, 1H), 1.60-1.34 (m, 6H), 1.05 (m, 1H). MS (ESI⁺) 489 (M⁺).

### EXAMPLE 64

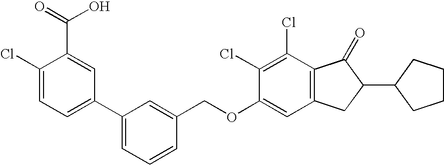

4-Chloro-3′-{([(6,7-dichloro-2-cyclopentyl-1-oxo-2,3-dihydro-1H-inden-5-yl)oxy]methyl}biphenyl-3-carboxylic acid

4-Chloro-3′-{([(6,7-dichloro-2-cyclopentyl-1-oxo-2,3-dihydro-1H-inden-5-yl)oxy]methyl}biphenyl-3-carboxylic acid was synthesized as described in general procedures A, B, C, and D using methyl 5-bromo-2-chlorobenzoate and 6,7-dichloro-2-cyclopentyl-5-hydroxyindan-1-one as starting materials. ¹H NMR (DMSO-d₆, 500 MHz) δ 13.53 (bs, 10H), 8.07 (d, 1H), 7.87 (s, 1H), 7.86 (d, 1H), 7.73 (m, 1H), 7.66 (d, 1H), 7.55 (m, 2H), 7.48 (s, 1H), 5.44 (s, 2H), 3.21 (m, 1H), 2.80 (m, 2H), 2.19 (m, 1H), 1.84 (m, 1H), 1.61-1.46 (m, 5H), 1.36 (m, 1H), 1.08 (m, 1H). MS (ESI⁺) 529 (M⁺).

### EXAMPLE 65

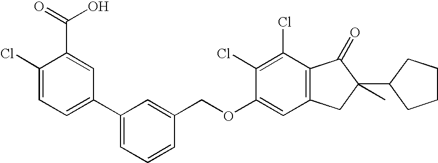

### 4-Chloro-3′-{[(6,7-dichloro-2-cyclopentyl-2-methyl-1-oxo-2,3-dihydro-1H-inden-5-yl)oxy]methyl}biphenyl-3-carboxylic acid

4-Chloro-3′-{[(6,7-dichloro-2-cyclopentyl-2-methyl-1-oxo-2,3-dihydro-1H-inden-5-yl)oxy]methyl}biphenyl-3-carboxylic acid was synthesized as described in general procedures A, B, C, and D using methyl 5-bromo-2-chlorobenzoate and 6,7-dichloro-2-cyclopentyl-5-hydroxy-2-methylindan-1-one as starting materials. ¹H NMR (MeOD, 500 MHz) δ 8.11 (s, 1H), 7.82 (s, 1H), 7.77 (d, 1H), 7.66 (m, 1H), 7.59 (d, 1H), 7.54 (m, 2H), 7.30 (s, 1H), 5.40 (s, 2H), 3.08 (d, 1H), 2.79 (d, 1H), 2.18 (m, 1H), 1.83 (m, 1H), 1.64-1.41 (m, 5H), 1.28 (m, 1H), 1.22 (s, 3H), 0.91 (m, 1H). MS (ESI⁺) 543 (M⁺).

### EXAMPLE 66

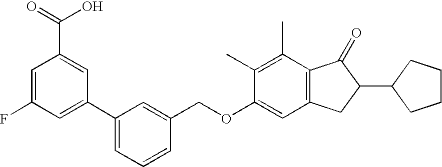

### 3′-{[(2-Cyclopentyl-6,7-dimethyl-1-oxo-2,3-dihydro-1H-inden-5-yl)oxy]methyl}-5-fluorobiphenyl-3-carboxylic acid

3′-{[(2-Cyclopentyl-6,7-dimethyl-1-oxo-2,3-dihydro-1H-inden-5-yl)oxy]methyl}-5-fluorobiphenyl-3-carboxylic acid was synthesized as described in general procedures A, B, C, and D using methyl 3-bromo-5-fluorobenzoate and 2-cyclopentyl-5-hydroxy-6,7-dimethylindan-1-one as starting materials. ¹H NMR (DMSO-d₆, 500 MHz) δ 13.44 (bs, 1H), 8.08 (s, 1H), 7.88 (s, 1H), 7.85 (d, 1H), 7.75 (m, 1H), 7.68 (d, 1H), 7.57 (d, 2H), 7.09 (s, 1H), 5.32 (s, 2H), 3.10 (m, 1H), 2.66 (m, 2H), 2.54 (s, 3H), 2.21 (m, 1H), 2.14 (s, 3H), 1.85 (m, 1H), 1.60-1.33 (m, 6H), 1.04 (m, 1H). MS (ESI⁺) 473 (M⁺+1).

### EXAMPLE 67

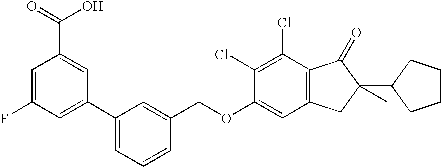

### 3′-{[(6,7-Dichloro-2-cyclopentyl-2-methyl-1-oxo-2,3-dihydro-1H-inden-5-yl)oxy]methyl}-5-fluorobiphenyl-3-carboxylic acid

3′-{[(6,7-Dichloro-2-cyclopentyl-2-methyl-1-oxo-2,3-dihydro-1H-inden-5-yl)oxy]methyl}-5-fluorobiphenyl-3-carboxylic acid was synthesized as described in general procedures A, B, C, and D using methyl 3-bromo-5-fluorobenzoate and 6,7-dichloro-2-cyclopentyl-5-hydroxy-2-methylindan-1-one as starting materials. ¹H NMR (DMSO-d₆, 500 MHz) δ 13.44 (bs, 1H), 8.10 (s, 1H), 7.93 (s, 1H), 7.86 (d, 1H), 7.79 (m, 1H), 7.69 (d, 1H), 7.58 (d, 2H), 7.48 (s, 1H), 5.45 (s, 2H), 3.02 (d, 1H), 2.79 (d, 1H), 2.06 (m, 1H), 1.75 (m, 1H), 1.55-1.42 (m, 4H), 1.33 (m, 1H), 1.20 (m, 1H), 1.15 (s, 3H), 0.86 (m, 1H). MS (ESI⁺) 527 (M⁺).

### EXAMPLE 68

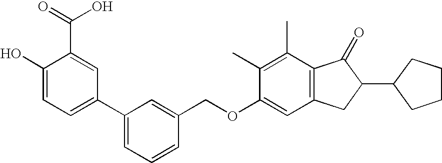

### 3′-{[(2-Cyclopentyl-6,7-dimethyl-1-oxo-2,3-dihydro-1H-inden-5-yl)oxy]methyl}-4-hydroxybiphenyl-3-carboxylic acid

3′-{[(2-Cyclopentyl-6,7-dimethyl-1-oxo-2,3-dihydro-1H-inden-5-yl)oxy]methyl}-4-hydroxybiphenyl-3-carboxylic acid was synthesized as described in general procedures A and D using 2-cyclopentyl-5-{[3-(5,5-dimethyl-1,3,2-dioxaborinan-2-yl)benzyl]oxy}-6,7-dimethylindan-1-one and methyl 5-bromo-2-hydroxybenzoate as starting material. ¹H NMR (DMSO-d₆, 500 MHz) δ 8.07 (s, 1H), 7.85 (d, 1H), 7.74 (s, 1H), 7.61 (d, 1H), 7.50 (t, 1H), 7.45 (d, 1H), 7.07 (m, 2H), 5.29 (s, 2H), 3.09 (m, 1H), 2.65 (m, 2H), 2.53 (s, 3H), 2.19 (m, 1H), 2.14 (s, 3H), 1.84 (m, 1H), 1.60-1.44 (m, 6H), 1.04 (m, 1H). MS (ESI⁺) 471 (M⁺+1).

### EXAMPLE 69

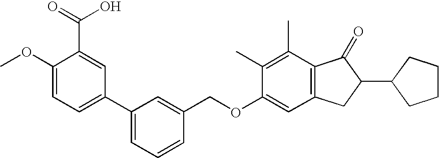

### 3′-{[(2-Cyclopentyl-6,7-dimethyl-1-oxo-2,3-dihydro-1H-inden-5-yl)oxy]methyl}-4-methoxybiphenyl-3-carboxylic acid

3′-{[(2-Cyclopentyl-6,7-dimethyl-1-oxo-2,3-dihydro-1H-inden-5-yl)oxy]methyl}-4-methoxybiphenyl-3-carboxylic acid was synthesized as described in general procedures A and D using 2-cyclopentyl-5-{[3-(5,5-dimethyl-1,3,2-dioxaborinan-2-yl)benzyl]oxy}-6,7-dimethylindan-1-one and methyl 5-bromo-2-methoxybenzoate as starting material. ¹H NMR (DMSO-d₆, 500 MHz) δ 12.75 (bs, 1H), 7.94 (s, 1H), 7.83 (d, 1H), 7.76 (s, 1H), 7.63 (d, 1H), 7.50 (t, 1H), 7.46 (d, 1H), 7.25 (d, 1H), 7.07 (s, 1H), 5.29 (s, 2H), 3.89 (s, 3H), 3.09 (m, 1H), 2.65 (m, 2H), 2.53 (s, 3H), 2.20 (m, 1H), 2.14 (s, 3H), 1.83 (m, 1H), 1.60-1.34 (m, 6H), 1.04 (m, 1H).

### EXAMPLE 70

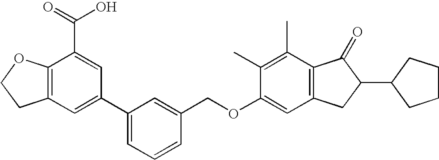

### 5-(3-{[(2-Cyclopentyl-6,7-dimethyl-1-oxo-2,3-dihydro-1H-inden-5-yl)oxy]methyl}phenyl)-2,3-dihydro-1-benzofuran-7-carboxylic acid

5-(3-{[(2-Cyclopentyl-6,7-dimethyl-1-oxo-2,3-dihydro-1H-inden-5-yl)oxy]methyl}phenyl)-2,3-dihydro-1-benzofuran-7-carboxylic acid was synthesized as described in general procedures A and D using 2-cyclopentyl-5-{([3-(5,5-dimethyl-1,3,2-dioxaborinan-2-yl)benzyl]oxy}-6,7-dimethylindan-1-one and methyl 5-bromo-2,3-dihydro-1-benzofuran-7-carboxylate as starting material. ¹H NMR (DMSO-d₆, 500 MHz) δ 12.75 (bs, 1H), 7.86 (s, 1H), 7.77 (s, 1H), 7.73 (s, 1H), 7.60 (d, 1H), 7.49 (t, 1H), 7.44 (d, 1H), 7.07 (s, 1H), 5.76 (s, 2H), 4.67 (t, 2H), 3.28 (t, 2H), 3.07 (m, 1H), 2.66 (m, 2H), 2.53 (s, 3H), 2.20 (m, 1H), 2.14 (s, 3H), 1.84 (m, 1H), 1.59-1.34 (m, 6H), 1.04 (s, 1H). MS (ESI⁺) 497 (M⁺+1).

### EXAMPLE 71

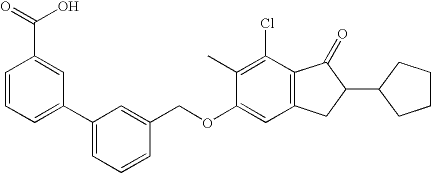

### 3′-{[(7-Chloro-2-cyclopentyl-6-methyl-1-oxo-2,3-dihydro-1H-inden-5-yl)oxy]methyl}biphenyl-3-carboxylic acid

3′-{[(7-Chloro-2-cyclopentyl-6-methyl-1-oxo-2,3-dihydro-1H-inden-5-yl)oxy]methyl}biphenyl-3-carboxylic acid was synthesized as described in general procedures A, B, C, and D using ethyl 3-bromobenzoate and 7-chloro-2-cyclopentyl-5-hydroxy-6-methylindan-1-one as starting materials. ¹H NMR (DMSO-d₆, 500 MHz) δ 8.23 (s, 1H), 7.95 (m, 2H), 7.85 (s, 1H), 7.71 (d, 1H), 7.67 (t, 1H), 7.55 (m, 2H), 7.27 (s, 1H), 5.37 (s, 2H), 3.16 (m, 1H), 2.75 (m, 2H), 2.27 (s, 3H), 2.19 (m, 1H), 1.85 (m, 1H), 1.60-1.34 (m, 6H), 1.06 (m, 1H). MS (ESI⁺) 475 (M⁺).

### EXAMPLE 72

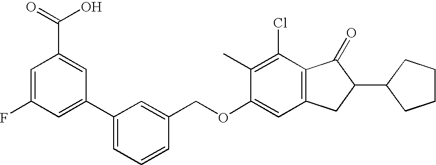

### 3′-{[(7-Chloro-2-cyclopentyl-6-methyl-1-oxo-2,3-dihydro-1H-inden-5-yl)oxy]methyl}-5-fluorobiphenyl-3-carboxylic acid

3′-{[(7-Chloro-2-cyclopentyl-6-methyl-1-oxo-2,3-dihydro-1H-inden-5-yl)oxy]methyl}-5-fluorobiphenyl-3-carboxylic acid was synthesized as described in general procedures A, B, C, and D using methyl 3-bromo-5-fluorobenzoate and 7-chloro-2-cyclopentyl-5-hydroxy-6-methylindan-1-one as starting materials. ¹H NMR (DMSO-d₆, 500 MHz) δ 13.51 (bs, 1H), 8.18 (s, 1H), 7.99 (s, 1H), 7.95 (d, 1H), 7.85 (m, 1H), 7.77 (d, 1H), 7.66 (m, 2H), 7.36 (s, 1H), 5.45 (s, 2H), 3.25 (m, 1H), 2.83 (m, 2H), 2.35 (s, 3H), 2.28 (m, 1H), 1.93 (m, 1H), 1.70-1.42 (m, 6H), 1.15 (m, 1H). MS (ESI⁺) 493 (M⁺).

### EXAMPLE 73

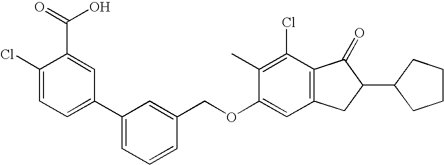

### 4-Chloro-3′-{[(7-chloro-2-cyclopentyl-6-methyl-1-oxo-2,3-dihydro-1H-inden-5-yl)oxy]methyl}biphenyl-3-carboxylic acid

4-Chloro-3′-{[(7-chloro-2-cyclopentyl-6-methyl-1-oxo-2,3-dihydro-1H-inden-5-yl)oxy]methyl}biphenyl-3-carboxylic acid was synthesized as described in general procedures A, B, C, and D using methyl 5-bromo-2-chlorobenzoate and 7-chloro-2-cyclopentyl-5-hydroxy-6-methylindan-1-one as starting materials. ¹H NMR (DMSO-d₆, 500 MHz) δ 13.39 (bs, 1H), 7.96 (s, 1H), 7.76 (s, 1H), 7.74 (s, 1H), 7.60 (m, 1H), 7.54 (d, 1H), 7.44 (m, 2H), 7.14 (s, 1H), 5.23 (s, 2H), 3.04 (m, 1H), 2.63 (m, 2H), 2.14 (s, 3H), 2.06 (m, 1H), 1.73 (m, 1H), 1.49-1.21 (m, 6H), 0.94 (m, 1H). MS (ESI⁺) 509 (M⁺).

### EXAMPLE 74

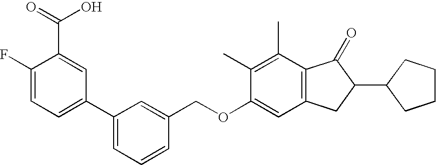

### 3′-{[(2-Cyclopentyl-6,7-dimethyl-1-oxo-2,3-dihydro-1H-inden-5-yl)oxy]methyl}-4-fluorobiphenyl-3-carboxylic acid

3′-{[(2-Cyclopentyl-6,7-dimethyl-1-oxo-2,3-dihydro-1H-inden-5-yl)oxy]methyl}-4-fluorobiphenyl-3-carboxylic acid was synthesized as described in general procedures A and D using 2-cyclopentyl-5-{[3-(5,5-dimethyl-1,3,2-dioxaborinan-2-yl)benzyl]oxy}-6,7-dimethylindan-1-one and methyl 5-bromo-2-fluorobenzoate as starting material. ¹H NMR (DMSO-d₆, 500 MHz) δ 13.45 (bs, 1H), 8.19 (m, 1H), 8.02 (m, 1H), 7.87 (s, 1H), 7.73 (d, 1H), 7.59 (m, 2H), 7.50 (t, 1H), 7.14 (s, 1H), 3.15 (m, 1H), 2.73 (m, 2H), 2.59 (s, 3H), 2.26 (m, 1H), 2.20 (s, 3H), 1.89 (m, 1H), 1.66-1.51 (m, 5H), 1.41 (m, 1H), 1.10 (m, 1H). MS (ESI⁺) 473 (M⁺+1).

### EXAMPLE 75

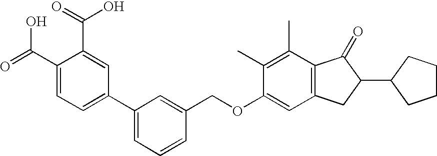

### 3′-{[(2-Cyclopentyl-6,7-dimethyl-1-oxo-2,3-dihydro-1H-inden-5-yl)oxy]methyl}biphenyl-3,4-dicarboxylic acid

3′-{[(2-Cyclopentyl-6,7-dimethyl-1-oxo-2,3-dihydro-1H-inden-5-yl)oxy]methyl}biphenyl-3,4-dicarboxylic acid was synthesized as described in general procedures A and D using 2-cyclopentyl-5-{[3-(5,5-dimethyl-1,3,2-dioxaborinan-2-yl)benzyl]oxy}-6,7-dimethylindan-1-one and dimethyl 4-bromophthalate as starting material. ¹H NMR (DMSO-d₆, 500 MHz) δ 7.72 (s, 1H), 7.69 (d, 1H), 7.66 (s, 1H), 7.61 (d, 1H), 7.54 (m, 1H), 7.34 (m, 2H), 6.87 (s, 1H), 5.10 (s, 2H), 2.88 (m, 1H), 2.45 (m, 2H), 2.31 (s, 3H), 1.98 (m, 1H), 1.92 (s, 3H), 1.61 (m, 1H), 1.38-1.23 (m, 5H), 1.14 (m, 1H), 0.82 (m, 1H). MS (ESI⁺) 499 (M⁺+1).

### EXAMPLE 76

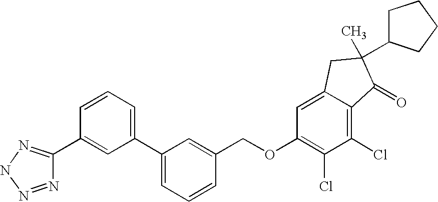

### 6,7-Dichloro-2-cyclopentyl-2-methyl-5-{[3′-(2H-tetrazol-5-yl)biphenyl-3-yl]methoxy}indan-1-one

6,7-Dichloro-2-cyclopentyl-2-methyl-5-[3′-(2H-tetrazol-5-yl)biphenyl-3-yl]methoxy)indan-1-one was synthesized as described in general procedures A, B, C, and F using 3-bromobenzonitrile and 6,7-dichloro-2-cyclopentyl-5-hydroxy-2-methyl indan-1-one as starting materials. MS (ESI⁺) 534.83 (M++1).

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention. For example, effective dosages other than the particular dosages as set forth herein above may be applicable as a consequence of variations in responsiveness of the mammal being treated for any of the indications with the compounds of the invention indicated above.

1-27. (canceled)

28. A compound of the formula I: 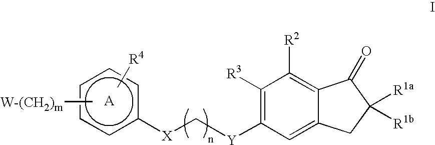
wherein:
A is phenyl or pyridyl;
W is selected from the group consisting of:
(1) tetrazolyl,
(2) CO₂H,
(3) NHSO₂C₁₋₁₆alkyl,
(4) NHSO₂-phenyl, wherein the phenyl is unsubstituted or substituted with C₁₋₆alkyl, and
(5) CONHCO—C₁₋₆alkyl;
X is selected from the group consisting of:
(1) —O—,
(2) —S—, and
(3) a bond;
(4) —O-phenyl-,
(5) —S-phenyl-, and
(6) -phenyl-;
Y is selected from the group consisting of:
(1) —O—,
(2) —NH(CO)—, and
(3) a bond;
R^{1a} and R^{1b} are independently selected from the group consisting of:
(1) hydrogen,
(2) C₁₋₆alkyl, which is unsubstituted or substituted with a substituent selected from:
(a) halogen,
(b) hydroxyl, and
(c) phenyl, wherein the phenyl is unsubstituted or substituted with 1-5 substituents independently selected from halogen, cyano, CF₃, hydroxyl, C₁₋₁₆alkyl, and OC₁₋₆alkyl,
(3) C₃₋₇cycloalkyl, which is unsubstituted or substituted with halogen, hydroxyl or phenyl, and
(4) phenyl, wherein the phenyl is unsubstituted or substituted with 1-5 substituents independently selected from halogen, hydroxyl, cyano, CF₃, C₁₋₁₆alkyl, and OC₁₋₁₆alkyl, wherein the C₁₋₆alkyl and OC₁₋₆alkyl are linear or branched and optionally substituted with 1-5 halogen;
R² is selected from the group consisting of:
(1) halogen,
(2) hydroxyl,
(3) OC₁₋₆alkyl, and
(4) C₁₋₆alkyl, which is unsubstituted or substituted with halogen, hydroxyl or phenyl;
R³ is selected from the group consisting of:
(1) halogen, and
(2) C₁₋₆alkyl, which is unsubstituted or substituted with halogen, hydroxyl or phenyl;
R⁴ may include multiple substituents and is independently selected from the group consisting of:
(1) hydrogen,
(2) halogen,
(3) C₁₋₆alkyl, and
(4) —O—C₁₋₆alkyl,
or R⁴ may be joined to the phenyl ring at an adjacent carbon to form a dihydrofuranyl ring;
m is an integer selected from 0, 1, 2 and 3;
n is an integer selected from 0, 1, 2, 3, 4, 5 and 6;
and pharmaceutically acceptable salts thereof.

29. The compound of claim 28 wherein A is phenyl.

30. The compound of claim 28 wherein A is pyridyl.

31. The compound of claim 28 wherein W is CO₂H.

32. The compound of claim 28 wherein X is a bond and Y is —O—.

33. The compound of claim 28 wherein X is —O-phenyl-.

34. The compound of claim 34 wherein X is -phenyl-.

35. The compound of claim 28 wherein R^{1a} is C₁₋₆alkyl.

36. The compound of claim 28 wherein R^{1a} is C₅₋₆cycloalkyl.

37. The compound of claim 28 wherein R^{1a} is phenyl.

38. The compound of claim 28 wherein R^{1b} is hydrogen.

39. The compound of claim 28 wherein R^{1b} is C₁₋₆alkyl.

40. The compound of claim 28 wherein R² is chloro and R³ is chloro.

41. The compound of claim 28 wherein R⁴ is hydrogen or bromo.

42. The compound of claim 28 wherein m is 0.

43. The compound of claim 28 wherein n is 1.

44. The compound of claim 28 wherein n is 2.

45. A compound which is selected from the group consisting of:
6,7-Dichloro-2-cyclopentyl-2-methyl-5-{3-[4-(2H-tetrazol-5-yl)-phenoxy]-propoxy}-indan-1-one;
6,7-Dichloro-2-cyclopentyl-2-methyl-5-{2-[4-(2H-tetrazol-5-yl)-phenoxy]-ethoxy}-indan-1-one;
6,7-Dichloro-2-cyclopentyl-2-methyl-5-[4-(2H-tetrazol-5-yl)-benzyloxy]-indan-one;
6,7-Dichloro-2-cyclopentyl-2-methyl-5-[5-(2H-tetrazol-5-yl)-pentyloxy]-indan-n-one;
6,7-Dichloro-2-cyclopentyl-5-[4-(2H-tetrazol-5-yl)-benzyloxy]-indan-1-one;
6,7-Dichloro-2-propyl-5-[4-(2H-tetrazol-5-yl)-benzyloxy]-indan-1-one;
6,7-Dichloro-2-cyclopentyl-2-methyl-5-[4-(2H-tetrazol-5-yl)-butoxy]-indan-1-one;
6,7-Dichloro-2-isopropyl-5-[4-(2H-tetrazol-5-yl)-benzyloxy]-indan-1-one;
6,7-Dichloro-2-cyclopentyl-2-methyl-5-[4-(2H-tetrazol-5-yl)-phenylethynyl]-indan-1-one;
6,7-Dichloro-2-cyclopentyl-2-methyl-5-{2-[4-(2H-tetrazol-5-yl)-phenyl]-ethyl}-indan-1-one;
6,7-Dichloro-2,2-dimethyl-5-[4-(2H-tetrazol-5-yl)-benzyloxy]-indan-1-one;
2-(6,7-Dichloro-2-cyclopentyl-2-methyl-1-oxo-indan-5-yloxy)-N-[4-(1H-tetrazol-5-yl)-phenyl]-acetamide;
6,7-Dichloro-2-cyclopentylmethyl-2-methyl-5-[4-(1H-tetrazol-5-yl)-benzyloxy]-indan-1-one;
6,7-Dichloro-2-cyclopentyl-2-methyl-5-[3-(1H-tetrazol-5-yl)-benzyloxy]-indan-1-one;
6,7-Dichloro-2-cyclopentyl-2-methyl-5-[3-(1H-tetrazol-5-yl)-propoxy]-indan-1-one;
4-(6,7-Dichloro-2-cyclopentyl-2-methyl-1-oxo-indan-5-yloxymethyl)-benzoic acid;
6,7-Dichloro-2-methyl-2-phenyl-5-[4-(1H-tetrazol-5-yl)-benzyloxy]-indan-1-one;
2-Butyl-6,7-dichloro-2-cyclopentyl-5-[4-(1H-tetrazol-5-yl)-benzyloxy]-indan-1-one;
N-[4-(6,7-Dichloro-2-cyclopentyl-2-methyl-1-oxo-indan-5-yloxymethyl)-benzoyl]-methanesulfonamide;
N-[4-(6,7-Dichloro-2-cyclopentyl-2-methyl-1-oxo-indan-5-yloxymethyl)-benzoyl]-4-methyl-benzenesulfonamide;
6,7-Dichloro-2-cyclopentyl-2-methyl-5-[4-(1H-tetrazol-5-yl)-phenyl]-indan-1-one;
3,5-dibromo-4-{[(6,7-dichloro-2-cyclopentyl-2-methyl-1-oxo-2,3-dihydro-1H-inden-5-yl)oxy]methyl}-N-(methylsulfonyl)benzamide;
N-acetyl-4-{[(6,7-dichloro-2-cyclopentyl-2-methyl-1-oxo-2,3-dihydro-1H-inden-5-yl)oxy]methyl}benzamide;
6,7-dichloro-2-cyclopentyl-2-methyl-5-{[5-(1H-tetrazol-5-yl)pyridin-2-yl]methoxy}indan-1-one;
6,7-dichloro-2-cyclopentyl-2-methyl-5-{4-[4-(2H-tetrazol-5-yl)phenoxy]butoxy} indan-1-one;
6,7-dichloro-2-cyclopentyl-2-methyl-5-{4-[3-(2H-tetrazol-5-yl)phenoxy]butoxy} indan-1-one;
3′-{[(2-Cyclopentyl-6,7-dimethyl-1-oxo-2,3-dihydro-1H-inden-5-yl)oxy]methyl}biphenyl-3-carboxylic acid;
5-(3-{[(2-Cyclopentyl-6,7-dimethyl-1-oxo-2,3-dihydro-1H-inden-5-yl)oxy]methyl}phenyl)nicotinic acid;
2-Cyclopentyl-6,7-dimethyl-5-({3-[5-(1H-tetrazol-5-yl)pyridin-3-yl]benzyl}oxy)indan-1-one;
6,7-dichloro-2-cyclopentyl-2-methyl-5-({3-[4-(2H-tetrazol-5-yl)phenoxy]benzyl}oxy)indan-1-one
6-chloro-2-cyclopentyl-2-methyl-5-({3-[4-(2H-tetrazol-5-yl)phenoxy]benzyl}oxy)indan-1-one;
2-cyclopentyl-6,7-dimethyl-5-{[3′-(2H-tetrazol-5-yl)biphenyl-3-yl]methoxy}indan-1-one;
3′-{[(2-Cyclopentyl-6,7-dimethyl-1-oxo-2,3-dihydro-1H-inden-5-yl)oxy]methyl}biphenyl-4-carboxylic acid;
3′-{[(6,7-Dichloro-2-cyclopentyl-2-methyl-1-oxo-2,3-dihydro-1H-inden-5-yl)oxy]methyl}biphenyl-3-carboxylic acid;
3′-{[(2-cyclopentyl-2,6,7-trimethyl-1-oxo-2,3-dihydro-1H-inden-5-yl)oxy]methyl}biphenyl-4-carboxylic acid;
3′-{[(2-cyclopentyl-2,6,7-trimethyl-1-oxo-2,3-dihydro-1H-inden-5-yl)oxy]methyl}biphenyl-3-carboxylic acid;
2-cyclopentyl-6,7-dimethyl-5-{[4′-(2H-tetrazol-5-yl)biphenyl-3-yl]methoxy}indan-1-one;
3-(4-{4-[(6,7-dichloro-2-cyclopentyl-2-methyl-1-oxo-2,3-dihydro-1H-inden-5-yl)oxy]butoxy}phenyl)propanoic acid;
3′-{[(6,7-Dichloro-2-cyclopentyl-2-methyl-1-oxo-2,3-dihydro-1H-inden-5-yl)oxy]methyl}biphenyl-4-carboxylic acid;
5-(3-{[(2-Cyclopentyl-6,7-dimethyl-1-oxo-2,3-dihydro-1H-inden-5-yl)oxy]methyl}phenyl)pyridine-2-carboxylic acid;
4-(3-{[(2-cyclopentyl-2,6,7-trimethyl-1-oxo-2,3-dihydro-1H-inden-5-yl)oxy]methyl}phenoxy)benzoic acid;
3′-{[(2-cyclopentyl-6,7-dimethyl-1-oxo-2,3-dihydro-1H-inden-5-yl)oxy]methyl}-N-(methylsulfonyl)biphenyl-3-carboxamide;
3′-{[(2-Cyclopentyl-6,7-dimethyl-1-oxo-2,3-dihydro-1H-inden-5-yl)oxy]methyl}-2-methylbiphenyl-3-carboxylic acid;
3′-{[(2-Cyclopentyl-6,7-dimethyl-1-oxo-2,3-dihydro-1H-inden-5-yl)oxy]methyl}-3-methylbiphenyl-4-carboxylic acid;
3′-{[(2-Cyclopentyl-6,7-dimethyl-1-oxo-2,3-dihydro-1H-inden-5-yl)oxy]methyl}-2-methylbiphenyl-4-carboxylic acid;
4-Chloro-3′-{[(2-cyclopentyl-6,7-dimethyl-1-oxo-2,3-dihydro-1H-inden-5-yl)oxy]methyl}biphenyl-3-carboxylic acid;
3′-{[(2-Cyclopentyl-6,7-dimethyl-1-oxo-2,3-dihydro-1H-inden-5-yl)oxy]methyl}-6-methylbiphenyl-3-carboxylic acid;
3′-{[(6,7-Dichloro-2-cyclopentyl-1-oxo-2,3-dihydro-1H-inden-5-yl)oxy]methyl}biphenyl-4-carboxylic acid;
3′-{[(6,7-Dichloro-2-isopropyl-1-oxo-2,3-dihydro-1H-inden-5-yl)oxy]methyl}biphenyl-4-carboxylic acid;
3′-{[(6,7-Dichloro-1-oxo-2-propyl-2,3-dihydro-1H-inden-5-yl)oxy]methyl}biphenyl-4-carboxylic acid;
5-({2-chloro-5-[4-(2H-tetrazol-5-yl)phenoxy]benzyl}oxy)-2-cyclopentyl-6,7-dimethylindan-1-one;
4-(3-{[(2-cyclopentyl-6,7-dimethyl-1-oxo-2,3-dihydro-1H-inden-5-yl)oxy]methyl}phenoxy)benzoic acid;
4-(3-{[(6,7-dichloro-2-cyclopentyl-2-methyl-1-oxo-2,3-dihydro-1H-inden-5-yl)oxy]methyl}phenoxy)benzoic acid;
3′-{[(6,7-Dichloro-2,2-dimethyl-1-oxo-2,3-dihydro-1H-inden-5-yl)oxy]methyl}biphenyl-4-carboxylic acid;
3′-{[(6,7-Dichloro-2-methyl-1-oxo-2-phenyl-2,3-dihydro-1H-inden-5-yl)oxy]methyl}biphenyl-4-carboxylic acid;
3′-{[(2-Butyl-6,7-dichloro-2-cyclopentyl-1-oxo-2,3-dihydro-1H-inden-5-yl)oxy]methyl}biphenyl-4-carboxylic acid;
3′-({[6,7-Dichloro-2-(cyclopentylmethyl)-2-methyl-1-oxo-2,3-dihydro-1H-inden-5-yl]oxy}methyl)biphenyl-4-carboxylic acid;
3′-{[(7-Chloro-2-cyclopentyl-6-methyl-1-oxo-2,3-dihydro-1H-inden-5-yl)oxy]methyl}biphenyl-4-carboxylic acid;
3′-{[(2-Cyclopentyl-6,7-dimethyl-1-oxo-2,3-dihydro-1H-inden-5-yl)oxy]methyl}-6-fluorobiphenyl-3-carboxylic acid
3′-{[(2-Cyclopentyl-6,7-dimethyl-1-oxo-2,3-dihydro-1H-inden-5-yl)oxy]methyl}-2-fluorobiphenyl-4-carboxylic acid;
3′-{[(2-Cyclopentyl-6,7-dimethyl-1-oxo-2,3-dihydro-1H-inden-5-yl)oxy]methyl}-6-methoxybiphenyl-3-carboxylic acid;
3′-{[(2-Cyclopentyl-6,7-dimethyl-1-oxo-2,3-dihydro-1H-inden-5-yl)oxy]methyl}-2,6-dimethoxybiphenyl-4-carboxylic acid;
3-Chloro-3′-{[(2-cyclopentyl-6,7-dimethyl-1-oxo-2,3-dihydro-1H-inden-5-yl)oxy]methyl}biphenyl-4-carboxylic acid;
4-Chloro-3′-{[(6,7-dichloro-2-cyclopentyl-1-oxo-2,3-dihydro-1H-inden-5-yl)oxy]methyl}biphenyl-3-carboxylic acid;
4-Chloro-3′-{[(6,7-dichloro-2-cyclopentyl-2-methyl-1-oxo-2,3-dihydro-1H-inden-5-yl)oxy]methyl}biphenyl-3-carboxylic acid;
3′-{[(2-Cyclopentyl-6,7-dimethyl-1-oxo-2,3-dihydro-1H-inden-5-yl)oxy]methyl}-5-fluorobiphenyl-3-carboxylic acid;
3′-{[(6,7-Dichloro-2-cyclopentyl-2-methyl-1-oxo-2,3-dihydro-1H-inden-5-yl)oxy]methyl}-5-fluorobiphenyl-3-carboxylic acid;
3′-{[(2-Cyclopentyl-6,7-dimethyl-1-oxo-2,3-dihydro-1H-inden-5-yl)oxy]methyl}-4-hydroxybiphenyl-3-carboxylic acid;
3′-{[(2-Cyclopentyl-6,7-dimethyl-1-oxo-2,3-dihydro-1H-inden-5-yl)oxy]methyl}-4-methoxybiphenyl-3-carboxylic acid;
5-(3-{[(2-Cyclopentyl-6,7-dimethyl-1-oxo-2,3-dihydro-1H-inden-5-yl)oxy]methyl}phenyl)-2,3-dihydro-1-benzofuran-7-carboxylic acid;
3′-{[(7-Chloro-2-cyclopentyl-6-methyl-1-oxo-2,3-dihydro-1H-inden-5-yl)oxy]methyl}biphenyl-3-carboxylic acid;
3′-{[(7-Chloro-2-cyclopentyl-6-methyl-1-oxo-2,3-dihydro-1H-inden-5-yl)oxy]methyl}-5-fluorobiphenyl-3-carboxylic acid;
4-Chloro-3′-{[(7-chloro-2-cyclopentyl-6-methyl-1-oxo-2,3-dihydro-1H-inden-5-yl)oxy]methyl}biphenyl-3-carboxylic acid;
3′-{[(2-Cyclopentyl-6,7-dimethyl-1-oxo-2,3-dihydro-1H-inden-5-yl)oxy]methyl}-4-fluorobiphenyl-3-carboxylic acid;
3′-{[(2-Cyclopentyl-6,7-dimethyl-1-oxo-2,3-dihydro-1H-inden-5-yl)oxy]methyl}biphenyl-3,4-dicarboxylic acid;
6,7-Dichloro-2-cyclopentyl-2-methyl-5-{[3′-(2H-tetrazol-5-yl)biphenyl-3-yl]methoxy}indan-1-one;
or a pharmaceutically acceptable salt thereof.

46. A pharmaceutical composition which comprises an inert carrier and a compound of claim 28 or a pharmaceutically acceptable salt thereof.

47. A method for potentiation of metabotorpic glutamate receptor activity in a mammal which comprises the administration of an effective amount of the compound of claim 28 or a pharmaceutically acceptable salt thereof.

48. A method for treating, controlling, ameliorating or reducing the risk of a neurological and psychiatric disorders associated with glutamate dysfunction in a mammalian patient in need of such which comprises administering to the patient a therapeutically effective amount of a compound of claim 28 or a pharmaceutically acceptable salt thereof.

49. A method for treating, controlling, ameliorating or reducing the risk of anxiety in a mammalian patient in need of such which comprises administering to the patient a therapeutically effective amount of a compound of claim 28 or a pharmaceutically acceptable salt thereof.

50. A method for treating, controlling, ameliorating or reducing the risk of depression in a mammalian patient in need of such which comprises administering to the patient a therapeutically effective amount of a compound of claim 28 or a pharmaceutically acceptable salt thereof.

51. A method for treating, controlling, ameliorating or reducing the risk of schizophrenia in a mammalian patient in need of such which comprises administering to the patient a therapeutically effective amount of a compound of claim 28 or a pharmaceutically acceptable salt thereof.

52. A method for treating, controlling, ameliorating or reducing the risk of epilepsy in a mammalian patient in need of such which comprises administering to the patient a therapeutically effective amount of a compound of claim 28 or a pharmaceutically acceptable salt thereof.
